SEQUENCE LISTING (61 sequences)

SEQ ID NO: 1 - protein, 412 aa, Plasmodium falciparum
MKFFVLFLLVTLNLFHISLEHNVEEEKCDYMNIFTKPLFYNCYVFKDHGNKIQQGCCKYYKAVRKGYERKSEELYKLSEEEYNKQYGNIKTDLNKNDLYSINDIIKNSISEKDMDEIKHLFPVIGEKLLNMNGQDGNINIKRSITDIDDLLVEFNNKHFNLKDIQVKILNGSTKNYDAYFNHIKADQNFIKKKEEILQKSPFYNKTCFKSLGGKNCNNGNFNHNNNNKAHEDDDNDDEDLNYELYEQYKEDDNLNSPSNNMNNIPNGNVNNNDNTINNIHNNSTNNDTYNNSVTYNHPQYTELEEDNINNKSSYYSFTQNFSIVNLFSQNPRKVMQKYLEFKHHLSSPSKEKMNGFLDREYYNTTKKLNEVLEDDLKKTGETIDQKGKKGFVSSFFDDLMSLIYFPKKNVEL

SEQ ID NO: 2 - protein, 109 aa, Plasmodium falciparum
MCFLKYFIFSLIWIFLYNEKAKDNSFLMNHNKREYNVPNGRILRLDTPEPWGDSETYEVVTKDKHGNLITTVFNKNAEALYFYINKKKPKEKNSSKPKNSKMRKKGQNN

SEQ ID NO: 3 - protein, 1165 aa, Plasmodium falciparum
MSCVLKKITVSEENICCYTGEEEILEKEKKKNARDLLVESKKEKNKKIEQKKQLPLREKLPTYFSQNSSNSLKNSNFDKKQFCESGTKYLIKYKIYNLIKNQFYQVDVHHASVSPSLDKKKLNSTKNIIREKNKQTNKKKKKLPKIPEKKKKDFLINIATNFRKNKPKKNSENSNKKKKKKNYTNQQAKKDKSNNNIINNSNSNINNNSNSNSNSNSNNNNNYYYYYYNNKNKNKNSNNNNNNNNNLKNKNKNNKRNRKGKKNIKKKIYIEKNSLTLPKTNRYCNYKYEEYYDISDEKMEATNYDETYCYDEELKNDSKTTIYDEGVSKKNIYNCDVNHIFDNFCNYFNCVDKKESMNDDTANSEITLNSNNIKTQDIIIDNNEKSFLYEKNRQQNINVYTQTSNKKSRWLCSTADDSHNEYLTEYEKKIDSYDNIDNNVHKDYSNIHKNVTIANNNNITLNYDTNDFSSYTTQPIQLFNDQENNTYDMGTNIVRKGKSLYEETRKISNALFEMYSTNVNKDNMNNNMNGNNNNNNNNNENVDKLCNKNQLEAPTPSLFTTYINQSHNYHSIVDKNIVLVQIKNFSQKIDEYISDEKIFRAQKLIDHVKKYIEFYINYYKKYNDKEVVEKLELYHEMHCMHKNIKYNINNLKVNIIMHFLNFFHLNDMYTILNDYTYYFSVDMVNSITSESITNSNENTNSINHSDINNMKTKNSIHSYAPSELGGLFNSSIKCYSIDESLNSSKMYLTPTLGNNNNNNINVSNNEAENIENVHKSDNLNNNNNNNEDDGVNVDSGINNNSKYNTDDSDIKYEDISSVIVDKIYHQQYSNCNMLNGNKTLINQKSINPDNSANRKNSNRHMVKIIQKYSKRFKKFRKSKQNSEGWIKENDKYLDLSHRVDKDNNISVHIRAKLPYEVNRILSILNETELSVNWAPFLTSAKKIKNLSRASAIITQLYEYPIIGKKESLMYCLGANSLEELGCIILCCKAPPEFNKDILFYENMCEKININKFGEIIKVKEIPIKFRKTYKEVTFFDYTLPEPVPKLDRQRAANLCFLLYPMNNGKSTVLELFLHFENEFKYTPIKMVTFFIKKIVKNMYENIIKSCRNYDLLYSEFLMNNAEFYIWLDDQIKRYMKGKNDSKLLYSISLESYDEPEHNEELDSKT

SEQ ID NO: 4 - protein, 1692 aa, Plasmodium falciparum
MKCINAEDKIQQDHEVLYKMFKSLRRHEDDKFINGSIKLKKKSLNEEINRFKDINDKYIFSNVKKKIKLEINGCIKIKRKGAHEEYYNQLVKKVLHGSIFIQNKNINNILDYIKYIKYIKLGGKISMVKKKKRTDKNEIISPLLSKENINKLRYLKTIYDVKKERNQKSPRKSSYNIERKEILDLSMNRYSHFQSEISNNYNLKNNNFYDSIKLVDNKMGKTKGNKEKGNKGKEEDKDKDKHKDKDKSKEKEKDINIYKTNGSIKNTLNNNKCSFVLSPSCNDKIRHVDELEKKMNTFKLNIPSSIINKFNKSNNTNAVENRGSIKYNLIKERQEFMYEPIRNNKEKHNKIHLKGTINYINNNNNNKKKNNNNNVHPTKFIFMGKKKKKIIEHEGTDDNYILGNEEKKKLLYMDQYNNETNKSHETNVLCGNKIYNKCGNKNIFLDHSVFLNYNKKVPKHISMSCSSKNKDMKKSTYDETMLSMSDVDDIDYFMYIKEKNAIKYKDYMNKPKTICQDKSSIEKEKSVLRIKKVEDQGDIKEKKSYMNDLKYKIDKKEPSNKENTSNIRNKKFSSVSRKLSKHFSLSRSGATFSMSNKKEKKSNKKKRTRFNKENRYMKKYLNKIMNMSILRRHFNNVNSSVYITHQKCFDKHEEHTKINEKKCKKGVIKTVDGKEDMNILNVNNNKEKITTKGNKNDHVGDSKDGNKNDHVGDSKDGNKNDHVGDSKEDNKNDHVGDSKEDNKNDHVGDSKDNNKNDHVGDSKDNNKNDHVGDSKDNNKNDHVGDSKEDNTIILSKEKNIKIGIYFEETCDIKQSNDKNISTTNEIKKMSSPVTNKLLTKKMGFPVTNKLLTKKEGMLKKFAISKEKMDITKKEGVVLSMKKSFSLKKDAEIHISNDDNVMDKNKVNSTIKSVQDILSKQLFKSKGNPLLINKKHVPKMDSTNKMIVDKKISFKSSLFKKDENKKTNYINSEKEEEKNVNMEKFLLNELKENYLNDERINKPGLSKEDDVDIKEPVLINEQVDLIKKEDQNICTNTEQEHEMMQEQKINEDEVNSKGDENTKKTENTINDKETIDIINKKNSSESVKEEIMEKEMLDEKDKNVPHYLECNNLHNMDENTKEIKVEKCNDYMTDNITSKNNNMIIKKINMKMLPMVTKKALPKSKALPILLKKNISIPKEKFTTNKKLSNESIDKTESSNIKMDEHKEEEKQIIKPSKSIHKIIKPGRHSFFNANKLLNSIEIKIGEHNSKIIEKEGPIKMFSSSTSIISDGKIDSIHKSDEEEKINDLKEDSNVNIYVKEKGKGKVFMKKGIPKLMMKFNAFSTKKMGINNLENNKKVNTLRSIKDDKILEKTNDTLEEINGTLEKTNGTLEETNGTLEKTNGTLEETSEISNIQHEEEVKNEKDATLQDDDDAGHTLKTTKAILMKKIPAKYKMNTVKMGDIKLPSHMNKFINKFHSVKILSTLKEFPVKKLIKKKDSNEENNSLEKIKMDKEKINDVDMEKEADEDVMKIEESVMNKKENDETEIEMNNNNEVDNNEIDNNKVDNNEIDNNKVDNNEIDNNKVDNNEIDNNKVDNNEIDNNKVDNNEIDNDKVDNISQDKSEISLNKIEEPNLVAESTTIEKEPIDKIIPRSFSKSLSIPMKVPTIKENINKAKPTFWNKPKGKMFFLTKKEKSISQAKEILRSLEKSNN

SEQ ID NO: 5 - protein, 88 aa, Plasmodium falciparum
MFTLISVCSYLFYTHKESLLNLIGLSENRNSRKNRRRKKKKGKLKQPFPEEKLCLSDSDMRRKKFILRTYSDVNFNVTCTTKKERNSI

SEQ ID NO: 6 - protein, 1213 aa, Plasmodium falciparum
MCNKYRLHLLNQINPTTNTSSYKQHQLNNDLYKLKKKMEKNKVLFNKDDKMDILSMDTLKLADEIKTKIIAQQLHTVLVLYLQILKEKKIEEKNINDDIGQYIENVIISALQLYSLIEKQTKDRKKCYIKKICQLCNTLSNILIEYIKNIKGIQVQHNSSKINENNQKLLIDSLENSNKLSKEKSTRSIESSKEKNENVFEGTSGKLNTVSYPCGCVHSQDMKIKEISLENEFSKGYRLMCLYSAQELNEQINYNKNIINNSFKTFQKKYCNVKLRDRRELIYKFQKILENKNIQFYDIYKGNVKSNIVSNQVNNNAQEFHTDLYDGCSYNSNHIIKNVEEKGIRHLINILLHTNKSITTFEINSQGKIDAIIQFLQKNEKKKDIILLNEYEYFFTNTDDYTQKEKNKNRRKYRDRENVTCNNRMVKRTYKNVDDNNNNLKEENENIYFYVGNKKKIVFDIKKKQKIVYEIDEKNSLLTKKKFLKDENNKDILLKVYDENDWSDENMEFCYITEKKEKEKIVIKKNKHGKKKTVYTNKNNELDNNVKNFIFYKKNIKNVKEINFYDDEEVILSLLKQNEERKKLEDTKDNYDIKNMENTISKDDNTLVCEKNKIPSIWNKAKNKTCTNDRDMYILEHKYVENKNDEINVESLDTYTNIITNRKEFLLKKHYLDNQQDISSFLKIDDYYYFKKNGNDSDTNKFNKYDNDYKMYETNVKGNMDDTKKCIGNKFDKMRNKKLDNIIAINDDDNNNNNNNYMNNVCLESYIDLYLEPKKKNEKEKKKKKKNYLINRMNTLISGDGKKNISEEYNMNDKEKIKIEDFLFNEYLKKVSVRSQSLKKKKEKKHEHEHEHEQEHEQEHEHEQVHEQEHKHKHEHEHKHKHKHEQEHKHKHEHEHEQVHEQEHNKIKKIIYDTKIDMKKEEDTQRKIENNHNIISNEKNDILQIHNIVKKDNKHMEINKCSKNNETHNNLPMSNDQKNNILDKNNKMIDKNLLKKKSSLQMNSFNSDKKLYDNDISNVKEKKIYNERKVSHENSMENQRYEDDKMGRKYLSEGFSAYVHVALCENTIISKKCIISCNINTKMLKIETKEKKFIIDMNKLHVQEIPTTYDNLAIIKLIIKKKNNNNSNCLLVESKNLGALQHLGDEIGWNYDKTGYSMDHKFNDEKEKKERRKTDDEHINNKPQLIDNSTVGFFDKIKLNKFLGKKKTEEKDI

SEQ ID NO: 7 - DNA, 20 bp, synthetic construct
  source 1..20: mol_type = other DNA
ttatattctcaattgaatgg

SEQ ID NO: 8 - DNA, 20 bp, synthetic construct
  source 1..20: mol_type = other DNA
cccttcaatagattcgcaca

SEQ ID NO: 9 - DNA, 24 bp, synthetic construct
  source 1..24: mol_type = other DNA
attgtatttatattctcaattgaa

SEQ ID NO: 10 - DNA, 24 bp, synthetic construct
  source 1..24: mol_type = other DNA
aaacttcaattgagaatataaata

SEQ ID NO: 11 - DNA, 24 bp, synthetic construct
  source 1..24: mol_type = other DNA
attgcccttcaatagattcgcaca

SEQ ID NO: 12 - DNA, 24 bp, synthetic construct
  source 1..24: mol_type = other DNA
aaactgtgcgaatctattgaaggg

SEQ ID NO: 13 - DNA, 35 bp, synthetic construct
  source 1..35: mol_type = other DNA
atagcggccgcatttattagatcatcatttgacag

SEQ ID NO: 14 - DNA, 30 bp, synthetic construct
  source 1..30: mol_type = other DNA
agttatcccgggactacttatcttctggtc

SEQ ID NO: 15 - DNA, 22 bp, synthetic construct
  source 1..22: mol_type = other DNA
tggaaatgtgggttctacacca

SEQ ID NO: 16 - DNA, 21 bp, synthetic construct
  source 1..21: mol_type = other DNA
tcttaacggaaatgggctcac

SEQ ID NO: 17 - DNA, 20 bp, synthetic construct
  source 1..20: mol_type = other DNA
ttttggacgcagttgctgac

SEQ ID NO: 18 - DNA, 23 bp, synthetic construct
  source 1..23: mol_type = other DNA
cctttaccgcggtcaagcgtaat

SEQ ID NO: 19 - DNA, 22 bp, synthetic construct
  source 1..22: mol_type = other DNA
ttcatatgcacctcctgaatgt

SEQ ID NO: 20 - DNA, 23 bp, synthetic construct
  source 1..23: mol_type = other DNA
agccataagggtttactattcca

SEQ ID NO: 21 - DNA, 20 bp, synthetic construct
  source 1..20: mol_type = other DNA
acgctgtacgtgtgtctagg

SEQ ID NO: 22 - DNA, 25 bp, synthetic construct
  source 1..25: mol_type = other DNA
tgcataattggattctacgatcagg

SEQ ID NO: 23 - DNA, 24 bp, synthetic construct
  source 1..24: mol_type = other DNA
ggggttgtaaaggaaaaagaagca

SEQ ID NO: 24 - DNA, 24 bp, synthetic construct
  source 1..24: mol_type = other DNA
tttctatcagtcgtttcagatcct

SEQ ID NO: 25 - DNA, 21 bp, synthetic construct
  source 1..21: mol_type = other DNA
tttgtacctccgaattcacca

SEQ ID NO: 26 - DNA, 24 bp, synthetic construct
  source 1..24: mol_type = other DNA
agtgtttgtatggaatgacaagtt

SEQ ID NO: 27 - DNA, 22 bp, synthetic construct
  source 1..22: mol_type = other DNA
agggataatggaactgtcgttt

SEQ ID NO: 28 - DNA, 23 bp, synthetic construct
  source 1..23: mol_type = other DNA
tctgttttcccaccaactaaaca

SEQ ID NO: 29 - DNA, 20 bp, synthetic construct
  source 1..20: mol_type = other DNA
tcatgcaactaccccctcgt

SEQ ID NO: 30 - DNA, 21 bp, synthetic construct
  source 1..21: mol_type = other DNA
tcaaagctcccttctcctcaa

SEQ ID NO: 31 - DNA, 23 bp, synthetic construct
  source 1..23: mol_type = other DNA
aagggagcaaatggtttaactgg

SEQ ID NO: 32 - DNA, 21 bp, synthetic construct
  source 1..21: mol_type = other DNA
gagcacctcctccaaattccc

SEQ ID NO: 33 - DNA, 21 bp, synthetic construct
  source 1..21: mol_type = other DNA
aagtagcaggtcatcgtggtt

SEQ ID NO: 34 - DNA, 22 bp, synthetic construct
  source 1..22: mol_type = other DNA
caaagtcatcaagttcggcaca

SEQ ID NO: 35 - DNA, 21 bp, synthetic construct
  source 1..21: mol_type = other DNA
gccttgaattttattgcccct

SEQ ID NO: 36 - DNA, 21 bp, synthetic construct
  source 1..21: mol_type = other DNA
acctgatgcaatggcagagta

SEQ ID NO: 37 - DNA, 21 bp, synthetic construct
  source 1..21: mol_type = other DNA
agaaagcactctcaagcctct

SEQ ID NO: 38 - DNA, 24 bp, synthetic construct
  source 1..24: mol_type = other DNA
tccgtttcttcattagcatttccg

SEQ ID NO: 39 - DNA, 20 bp, synthetic construct
  source 1..20: mol_type = other DNA
aggaggggcaagtgcatatt

SEQ ID NO: 40 - DNA, 22 bp, synthetic construct
  source 1..22: mol_type = other DNA
actttgcgctacataactacca

SEQ ID NO: 41 - DNA, 20 bp, synthetic construct
  source 1..20: mol_type = other DNA
cttcgcttttgcaaacctgg

SEQ ID NO: 42 - DNA, 21 bp, synthetic construct
  source 1..21: mol_type = other DNA
tgaaggggatccttttccagc

SEQ ID NO: 43 - DNA, 23 bp, synthetic construct
  source 1..23: mol_type = other DNA
tgcatccaaacaaatgaagagga

SEQ ID NO: 44 - DNA, 24 bp, synthetic construct
  source 1..24: mol_type = other DNA
attggtatgcactggtatgttttt

SEQ ID NO: 45 - DNA, 24 bp, synthetic construct
  source 1..24: mol_type = other DNA
aggtgcagaatttcaagaaatgga

SEQ ID NO: 46 - DNA, 24 bp, synthetic construct
  source 1..24: mol_type = other DNA
ttttgagaaaacattctgtgttgc

SEQ ID NO: 47 - DNA, 21 bp, synthetic construct
  source 1..21: mol_type = other DNA
ccattcgagctctgagtctgt

SEQ ID NO: 48 - DNA, 20 bp, synthetic construct
  source 1..20: mol_type = other DNA
ccccacctcttccatatccg

SEQ ID NO: 49 - DNA, 21 bp, synthetic construct
  source 1..21: mol_type = other DNA
atgcaccgataagcattacac

SEQ ID NO: 50 - DNA, 29 bp, synthetic construct
  source 1..29: mol_type = other DNA
tatgatgtgaatatgtataattccccgtc

SEQ ID NO: 51 - DNA, 21 bp, synthetic construct
  source 1..21: mol_type = other DNA
caattctgcaagcatttcaat

SEQ ID NO: 52 - DNA, 19 bp, synthetic construct
  source 1..19: mol_type = other DNA
cttggatgagagcaccgaa

SEQ ID NO: 53 - DNA, 21 bp, synthetic construct
  source 1..21: mol_type = other DNA
taatatgtacgcaaggtgcac

SEQ ID NO: 54 - DNA, 23 bp, synthetic construct
  source 1..23: mol_type = other DNA
atttctgcactaaggatgtatta

SEQ ID NO: 55 - DNA, 23 bp, synthetic construct
  source 1..23: mol_type = other DNA
caaatcattctttaccttatgtt

SEQ ID NO: 56 - DNA, 22 bp, synthetic construct
  source 1..22: mol_type = other DNA
tttatctttttgtgcacagacc

SEQ ID NO: 57 - DNA, 23 bp, synthetic construct
  source 1..23: mol_type = other DNA
cttataagaacgaaataagtgtg

SEQ ID NO: 58 - DNA, 23 bp, synthetic construct
  source 1..23: mol_type = other DNA
tgctcaatataaaataagcactg

SEQ ID NO: 59 - DNA, 15 bp, synthetic construct
  source 1..15: mol_type = other DNA
ccgttgattcgtgca

SEQ ID NO: 60 - DNA, 22 bp, synthetic construct
  source 1..22: mol_type = other DNA
atacatatatgcatatgcactc

SEQ ID NO: 61 - DNA, 20 bp, synthetic construct
  source 1..20: mol_type = other DNA
tatttatattctcaattgaa

(19) United States
(12) Patent Application Publication
(10) Pub. No.: US 2025/0009862 A1
(43) Pub. Date: Jan. 9, 2025

(54) COMPOSITIONS AND METHODS FOR REDUCING PARASITIC INFECTIONS

(71) Applicant: King Abdullah University of Science and Technology, Thuwal (SA)

(72) Inventors: Amit Kumar Subudhi, Thuwal (SA); Arnab Pain, Thuwal (SA)

(21) Appl. No.: 18/739,100

(22) Filed: Jun. 10, 2024

Related U.S. Application Data
(60) Provisional application No. 63/507,027, filed on Jun. 8, 2023.

(51) Int. Cl. A61K 39/015; A61K 39/00

(52) U.S. Cl. CPC A61K 39/015; A61K 2039/522; A61K 2039/53

(57) ABSTRACT

Parasitic egress and invasion-associated genes and their encoded proteins (referred to as “EIAGs,”) and compositions and methods of use there, including in vaccines and targets for treatment of parasitic infections are provided.

### CROSS REFERENCE TO RELATED APPLICATIONS

This application claims the benefit of and priority to U.S. Provisional Application No. 63/507,027 filed Jun. 8, 2023, which is hereby incorporated herein by reference in its entirety.

### REFERENCE TO SEQUENCE LISTING

The Sequence Listing XML submitted as a file named “KAUST_2021_116_02_2022_090_01_US.xml” created on Sep. 23, 2024, and having a size of 59,507 bytes is hereby incorporated by reference pursuant to 37 C.F.R. § 1.834(c)(1).

### FIELD OF THE INVENTION

This invention is generally in the field of strategies for treating and preventing parasitic infections particularly those cause by P. falciparum, such as malaria.

### BACKGROUND OF THE INVENTION

Malaria is caused by single-celled apicomplexan parasites of the genus Plasmodium. All malaria-related pathology results from the ability of these parasites to invade, replicate within and egress from host red blood cells (RBCs). To invade and egress successfully, two essential processes in parasite biology, the parasite uses multiple proteins that are released sequentially from specialized parasite-specific apical organelles called exonemes, micronemes, rhoptries, and dense granules. While previous studies have identified arrays of essential and non-essential proteins that take part in the intricate processes of invasion and egress, there is not a complete picture of all the components of the invasion and egress machinery or their essentiality in the parasite lifecycle. With the widespread emergence of resistance to almost all known antimalarial drugs (1, 2) including mainstay artemisinin derivatives (3-6), there is broad agreement on the need to identify new drug targets that are essential for parasite survival. Crucially, in order to prevent cross-resistance with existing therapeutics, it is important that new drugs have new modes of action. A well accepted strategy to control malaria parasites is to simultaneously target proteins involved in multiple independent essential steps in parasite biology, so there is a need to identify new essential components of parasite pathways through improved understanding of parasite biology during key phases of the erythrocytic parasite life cycle.

Many of the known proteins involved in egress and invasion have been identified as druggable targets (e.g., plasmepsin IX and X, PKG, PKA, MyoA and SUB1) (7). However, many proteins have unassigned functionality. Hypothetical proteins of Plasmodium falciparum are shown in the Examples below to be tightly coexpressed with other well-characterized invasion and egress associated proteins. It is believed that these proteins likely represent previously unrecognized missing components of the egress and invasion pathways. As discussed in more detail below, they, and their orthologs in other parasites, are provided herein as targets for therapeutic interventions, including druggable inhibition and vaccine candidates, against parasites including, but not limited to, P. falciparum.

Invasion and egress are key steps in the blood-stage malaria parasite life cycle Malaria in humans is caused by seven species of Plasmodium: Plasmodium falciparum: P. vivax, P. malariae, P. ovale curtisi, P. ovale wallikeri (8), P. knowlesi (9) and P. simium (10). P. falciparum is responsible for the most debilitating form of the disease and accounts for most of the malaria-related mortality worldwide (11). In 2018, an estimated 228 million cases of malaria were reported worldwide, with an estimated 405,000 deaths from the disease (11). The asexual blood-stage cycle that is responsible for all malaria-related pathogenesis starts with the invasion of RBCs by merozoites, which are exquisitely evolved to perform this function (12, 13). Upon invasion, the parasite forms and resides within a membrane-bound parasitophorous vacuole (PV) and subsequently morphs through ring, trophozoite and multinucleated schizont stages that eventually undergo segmentation to produce 16-32 daughter merozoites. These merozoites are then released into the bloodstream through a lytic process called egress, rapidly invading fresh RBCs. Invasion is initiated by attachment of the merozoite to the RBC surface membrane (RBCM), possibly through merozoite surface proteins (MSPs) (FIGS. 1K-1M). Immediately following initial attachment, the parasite reorients to form a tight interaction with the RBC mediated through two classes of adhesins released from apical secretory organelles called micronemes and rhoptry necks (13). In P. falciparum, these adhesins include the erythrocyte-binding-like (EBL) proteins (members comprise EBA-175, EBA-181, EBA-140, and EBA1) and reticulocyte-binding-like (Rh) protein homologs (PfRh1, PfRh2a, PfRh2b, PfRh4, and PfRh5) respectively, which bind to a range of host cell receptors. Of these adhesins, PfRh5 plays an essential role in invasion (14, 15) by forming a complex with PfRiPR (P. falciparum Rh5 interacting protein) and CyPRA (cysteine-rich protective antigen); this complex is probably required to create a pore at the junction between the merozoite and RBCM (14, 16, 17). Following this, a further microneme adhesin called apical membrane antigen 1 (AMA1) is released to form a complex with rhoptry neck protein (RON) 2, a part of a larger RON complex, to facilitate formation of a so-called tight junction between parasite and host RBC (18, 19). Entry into the nascent PV then occurs, with motility powered by the parasite actinomyosin-based motor or glideosome, a large molecular machine associated with the inner membrane complex (IMC) and made up of glideosome associated proteins (GAPs), myosin A (MyoA), and a myosin A tail domain interacting protein (MTIP) amongst other proteins. The glideosome, in association with the adhesins, propels the parasite into the host cell (13).

The egress pathway is initiated following formation of individual daughter merozoites by segmentation of the mature schizont. The first step appears to be a change in the permeability of the PV membrane (PVM) (FIG. 1L), which allows intermingling of the cellular contents of the PV and host RBC cytosol. Shortly following this the single parasite cGMP-dependent protein kinase PKG is activated, presumably by an increase in cytosolic cGMP levels. Activated PKG, in association with a calcium-dependent protein kinase called CDPK5, triggers the discharge of subtilisin-like protease 1 (SUB1) from exonemes (20) into the PV lumen, where SUB1 proteolytically modifies a range of merozoite surface and PV proteins (21-23). In addition to its essentiality for rupturing the PVM24, SUB1 proteolytically activates a putative cysteine protease called SERA6 (25-27) required for RBCM rupture (24). MSP1, the most abundant merozoite surface protein, is also a substrate of SUB1 and participates in merozoite egress (28). Within a few minutes of SUB1 release, the PVM is fragmented, quickly followed by poration and rupture of the RBCM to release the merozoites. Each year, several new proteins are characterized that participate in parasite invasion, and egress, indicating that a full picture of these pathways is not yet known. This is important, because many of the proteins and processes involved (including several of the enzymes already identified) could represent excellent drug targets (24, 29-31), particularly if suitable new compounds could be delivered in combination with already licensed antimalarial drugs.

Thus, it is object of the invention to provide targets for interventions including, but not limited to, therapeutics and vaccines, and compositions and methods thereto for the treatment of parasitic infections.

### SUMMARY OF THE INVENTION

Parasitic egress and invasion-associated genes and their encoded proteins (referred to as “EIAGs,”) and compositions and methods of use thereof, including in vaccines and as targets for treatment of parasitic infections are provided. See, e.g., Tables 1 and 2.

An immunogenic composition can include, for example, an effective amount of (i) a protein encoded by any of the genes of Table 1 or Table 2, a variant thereof with at least 75% sequence identity thereto, or a fragment thereof; or (ii) a nucleic acid encoding the protein or variant or fragment of (i). In some embodiments, the nucleic acid is mRNA or a vector. The immunogenic composition can further include, for example, a delivery vehicle, adjuvant, an adjuvant, etc. Methods of inducing an immune response in a subject in need thereof are also provided and typically include administering the subject an effective amount of an immunogenic composition induce an immune response against the protein or variant or fragment.

Pharmaceutical compositions include an effective amount of an inhibitor of (i) a protein encoded by any of the genes of Table 1 or Table 2, a variant thereof with at least 75% sequence identity thereto, or a fragment thereof; or (ii) a nucleic acid encoding the protein or variant or fragment of (i), are also provided. In some embodiments, the inhibitor is a functional nucleic acid, an expression vector encoding a functional nucleic acid, or a small molecule. Methods of treating a subject in need thereof are also provided can include administering the subject an effective amount of the inhibitor composition to reduce one or more symptoms of a parasitic infection. In some embodiments, the subject has malaria. In some embodiments, the method further includes administering the subject second antimalarial agent, which optionally can be included in the same or a different composition. In some embodiments, the subject has a Plasmodium falciparum infection.

The compositions in some forms include live attenuated ΔPFAP2-MRP (herein, LA-APFAP2-MRP) Plasmodiun parasites or an orthologue thereof, such as P. malariae, P. vivax, Plasmodium falciparum, P. ovale; and P. knowlesi, which express multiple PfEMP1 proteins on the surface of infected RBCs. In some preferred embodiments, the compositions additionally include an adjuvant and optionally, an excipient.

In some forms, the compositions include sporozoite stage LA-APFAP2-MRP Plasmodium. In some forms the compositions include merozoites stage LA-APFAP2-MRP Plasmodium. In some forms the LA-APFAP2-MRP Plasmodium is a chemically attenuated LA-APFAP2-MRP Plasmodium. In some forms the LA-APFAP2-MRP Plasmodium is a genetically attenuated LA-APFAP2-MRP Plasmodium. In some forms the LA-APFAP2-MRP Plasmodium has been irradiated to provide attenuation.

Methods for eliciting an immune response against a malaria parasite are disclosed. The methods include administering the LA-APFAP2-MRP malaria parasite to the subject. In one embodiment, the administration is not followed by administration of an antimalarial agent that is capable of preventing/blocking parasite growth.

### BRIEF DESCRIPTION OF THE DRAWINGS

FIGS. 1A-1E show Conditional knockout of PfAP2-MRP and confirmation. FIG. 1A is an illustration of a CRISPR Cas9 based approach used to generate a floxed second exon of PfAP2-M in the II-3 parasite line (Knuepfer et al., 2017). Two batches of CRISPR Cas9 based experiments were performed to achieve this. In the first batch of CRISPR Cas9 experiment, the single intron of PfAP2-M was replaced with sera2 loxPint. In the second batch of CRISPR Cas9 experiment, a parasite clone with integrated sera2 loxPint was used and the 3′ end of the PfAP2-M was replaced with either a recodonized coding sequence fused with sequence coding for a triple HA tag, a loxP followed by a stop codon or a recodonized coding sequence with a stop codon followed by a loxP (without a triple HA). Two types of parasite lines were generated that have a floxed second exon of PfAP2-M from which a PfAP2-M:loxP or a PfAP2-M-HA3:loxP fusion proteins are expressed. Only the parasite line with HA tagged AP2-M is shown in the last two lines of the schematic. In the presence of rapamycin the floxed second exon is excised to produce an inactive truncated PfAP2-M protein. FIG. 1B. Diagnostic PCR with primers binding to upstream of homologous region 1 (HR1) and downstream of homologous region 4 (HR4) to detect successful excision of floxed second exon of PfAP2-MRP in the presence of rapamycin. Amplified bands of 3382 bp and 1172 bp were expected from the floxed (RAPA−) and excised (RAPA+) PfAP2-MRP-HA3 locus, respectively when primer pair P3 and P4 were used. FIG. 1C. Coverage plot of RNA-seq reads mapped to PfAP2-MRP locus and viewed in IGV browser. As a comparison, RNA-seq reads mapped to both the exons in mock-treated parasites grown in presence of only DMSO (RAPA−). FIG. 1D. Western blot with anti-HA antibodies probed against schizont extracts of mock-treated (RAPA−) and rapamycin-treated (RAPA+) PfAP2-MRP-HA3:loxP parasites. Reactivity of antibodies to histone 3 was used as a loading control. FIG. 1E. Light microscopic images of mature schizonts at cycle 0, showing normal parasite development. Image of schizonts for mock-treated (RAPA−) parasites was captured at 47 h.p.i. whereas for rapamycin-treated (RAPA+) parasites, images were captured both at 47 h.p.i. and 49 h.p.i. but the only image from 49 h.p.i. is shown here (representative of 4 independent experiments). FIG. 1F. Invasion by mock (RAPA−) and rapamycin-treated (RAPA+) PfAP2-MRP-HA3:loxP parasites under static and shaking conditions. Statistical significance by two-tailed t-test: RAPA−versus RAPA+parasites in static condition (t=14.17, d.f.=4, P=0.000013, 95% CI 1.606 to 2.389) n=3 and RAPA−versus RAPA+parasites in shaking condition (t=12.41, d.f.=4, P=0.000021, 95% CI 1.578 to 2.487) n=3. FIG. 1G. Rapamycin was added to synchronized PfAP2-MRP parasite cultures at 16 h.p.i. Mock-treated schizonts were purified when egress was detectable in the culture, and rapamycin-treated schizonts were harvested 2 hours thereafter. Thin films of purified schizonts were made and prepared for indirect immunofluorescence. GAP45 was detected by incubation with rabbit anti-GAP45 IgG, followed by anti-rabbit IgG conjugated with Alexa 488 (labelled in green). In RAPA(−) parasites GAP45 localizes to the periphery of individual merozoites, whereas in RAPA(+) parasites the protein appears disorganized by comparison. FIG. 1H. MSP7 was detected by incubation with rabbit anti-MSP7 serum, followed by anti-rabbit IgG conjugated with Alexa 488 (labelled in green). In RAPA(−) parasites MSP7 localizes to the periphery of individual merozoites, whereas in RAPA(+) parasites, although the staining appears defined, there is no envelopment of merozoites. In all cases, the scale bar is 2 μm. FIGS. 1I and 1J show Immunofluorescence microscopy of control [RAPA (−)] and treated [RAPA (+)] parasites incubated with anti-GAP45 IgG (FIG. 1I) and anti-MSP7 serum (FIG. 1J); scale bar is 2 μm. FIGS. 1K-1M are illustrations of the Subcellular structure of the merozoite and steps of malaria parasite invasion and egress. FIG. 1K is an illustration of the architecture of a P. falciparum blood stage merozoite, showing the specialized secretory apical organelles. FIG. 1L is an illustration of major stages of invasion of the RBC (initial attachment, reorientation leading to apical interaction, and invasion) and the proteins known to be involved in each stage of invasion. Initial attachment between merozoite and RBC is low-affinity, likely involving interaction between merozoite surface proteins (MSPs) and receptors on the RBC surface. Tight interaction takes place following initial attachment, involving EBAs and Rhs. The merozoite then reorients so that its apical end is adjacent to the RBC, facilitating tight attachment. A pore is probably formed between the RBC and the apical end of the merozoite, mediated by the PfRH5-PfRipr-PfCyRPA complex. This is followed by formation of an irreversible tight junction between the parasite ligand AMA1 (secreted from micronemes) and RON2, a rhoptry neck protein that is inserted into the RBC membrane. The merozoite then moves into the nascent PV through the tight junction, motility being powered by the parasite's actinomyosin motor. During entry the MSP surface coat is shed through the activity of a serine protease or “sheddase’ called SUB2. Once the posterior pole of the merozoite enters the nascent PV, adhesive proteins at the junction between the parasite and RBC are also removed proteolytically. This probably facilitates resealing of the RBC membrane, enabling pinching off of the vacuole. FIG. 1L is adapted and modified from Cowman et al., 2017 (13). FIG. 1M is an illustration of the stages of parasite egress. After schizont segmentation, the PVM becomes permeable allowing mixing of the contents of the PV and residual RBC cytoplasm. The subtilisin like protease SUB1, maturation of which requires an aspartic protease called plasmepsin X, is then triggered by activated PKG to be released from the parasite's exonemes. SUB1 is required for PVM rupture, and modifies and activates many proteins including SERA6. Activated SERA6 mediates RBCM disruption, enabling release of the daughter merozoites. FIG. 1N shows RNA-seq reads coverage from 16 h.p.i. parasites (cycle 1) of pf-ap2-mrp locus. FIG. 1O shows images of Giemsa-stained parasites at 39, 43, 46 and 49 h.p.i. stages in cycle 1 following parasite treatment with DMSO [RAPA (−)] or rapamycin [RAPA(+)] at 35 h.p.i. at cycle 0.

FIGS. 2A and 2B are volcano plots showing differentially expressed var genes in 16 h.p.i (cycle 1) (FIG. 2A) and 40 h.p.i. (cycle 0) (FIG. 2B) Apfap2-mrp parasites. FIGS. 2C-2D. Differential expression of var genes (log 2 ratio of Apfap2-mrp to mock-treated control parasites) measured by qRTPCR at 16 h.p.i. (FIG. 2C) and 40 h.p.i. (FIG. 2D); error bar is s.e.m. FIG. 2E is a lollipop plot of expression level of top 20 significantly down-regulated genes in Δpfap2-mrp compared to control parasites at 40 h.p.i. FIG. 2F shows heatmaps for most down-regulated genes known to be involved in parasite egress and invasion in treated [RAPA (+)] or control [RAPA (−)] parasites, grouped based on the sub-cellular location of their products. FIG. 2G shows western blots of schizont extract from parental II-3 and PfAP2-MRP-3HA parasites in the absence (−) or presence (+) of rapamycin, probed with antibodies specific for invasion proteins. BiP was detected as a loading control. Molecular mass (kDa) of standards on left side of each panel. A non-specific cross-reacting protein on the SUB1 blot is marked with an asterisk. FIG. 2H. Left: uniform manifold approximation and projection (UMAP) of scRNA-seq data from Malaria Cell Atlas (MCA), with annotated developmental stages. Right: UMAP projections of scRNA-seq in-house data; each dot represents gene expression data from a single parasite (colors corresponding to h.p.i. and pfap2-mrp knockout status) plotted over MCA data. FIG. 2I shows distribution of developmental stages of treated [RAPA(+)] or control [RAPA(−)] parasites at 16 and 40 h.p.i. FIG. 2J shows violin plots of average var gene expression in treated [RAPA (+)] or control [RAPA (−)] parasites at both 16 h.p.i. and 40 h.p.i. FIG. 2K shows proportion of cells expressing one or more var genes from treated [RAPA(+)] or control [RAPA (−)] cultures (P=6.23e-13, Fisher's exact test, odds ratio=1.53). FIG. 2L shows percentage of iRBCs containing control or Apfap2-mrp parasites bound by IgG from serum of malaria-infected (HS+) patients, or untreated samples (HS−). Significance determined using a two-tailed t-test (t=6.687, d.f.=4, P<0.0001, 95% CI 0.9844 to 2.382, n=3).

FIG. 3A shows genome-wide occupancy of PfAP2-MRP at 16 h.p.i. and 40 h.p.i., determined by ChIP-seq. Sub-telomeric and internal regions of chromosome 7 (˜1450 kb) containing var genes with PfAP2-MRP bound in their promoter regions are shown as an example. Chromosomal positions are indicated. Results are representative of 2 independent replicates. FIG. 3B shows pie charts showing the proportion of each family of genes with PfAP2-MRP bound to the promoter region (in blue) at 16 h.p.i. FIG. 3C shows PfAP2-MRP occupancy at 16 h.p.i. in putative promoter regions of genes implicated in iRBC remodeling and parasite development. Two biological replicate ChIP vs. Input tracks are shown, (input subtracted PfAP2-MRP-ChIP). Positions on Chr 09, 10, 11 and 13 are indicated. X-axis shows the genomic position and numbers on the right, show the enrichment score (see Methods in Examples Section). FIG. 3D shows the expression levels of 27 P. falciparum AP2 genes during different IDC stages and in merozoites are depicted by the diameter of the circles. On the right, blue, brown and green circles indicate the binding of PfAP2-MRP to the promoter at either or both 16 h.p.i. and 40 h.p.i.; the heatmap displays the expression status of all 26 api-ap2s in Δpfap2-mrp parasites compared to controls at 16 and 40 h.p.i. The heatmap for pfap2-mrp is black because Δpfap2-mrp parasites only express RNA from the first exon and have no functional AP2-MRP protein.

FIG. 4A shows the most significantly enriched motif bound by PfAP2-MRP at 16 h.p.i. FIGS. 4B and 4C show the two most significantly enriched motifs bound by PfAP2-MRP at 40 h.p.i. FIG. 4D shows the most enriched motif (panel c) at 40 h.p.i. is similar to the PfAP2-I binding motif FIG. 4E shows the numbers of genes with promoters bound by PfAP2-MRP at either 16 h.p.i., 40 h.p.i. or both. FIGS. 4F and 4G show the Genomic regions uniquely bound by PfAP2-MRP at 16 h.p.i. (FIG. 4F) or at both 16 and 40 h.p.i. (FIG. 4G). X-axis shows the genomic position and numbers on the right, show the enrichment score. FIGS. 4H and 41 show the Label-free quantitative proteomic analysis of P. falciparum proteins enriched in PfAP2-MRP immunoprecipitates at 16 h.p.i. and 40 h.p.i.

FIG. 5A shows a chromatin contact count heatmap of chromosome 7 at 16 h.p.i. (top) and 40 h.p.i (bottom) for the WT (left) and Δpfap2-mrp (middle), as well as the log_2 fold change in interactions (right) between the WT and Δpfap2-mrp. Blue indicates a loss of interactions and red indicates an increase of interactions Δpfap2-mrp over WT. FIG. 5B shows Whole-genome interchromosomal contact count heatmaps at 16 h.p.i. for the WT (left) and Δpfap2-mrp (right). Chromosomes are sorted from left to right and bottom to top. Intrachromosomal interactions are removed. FIG. 5C shows Whole-genome 3D chromatin models at 16 h.p.i. for the WT (left) and Δpfap2-mrp (right). Centromeres (blue), telomeres (green), and var genes (red) are enhanced to display differences between the two samples. FIG. 5D shows the Number of interactions between var gene containing bins.

FIG. 6A is a line plot showing the expression pattern of PfAP2-MRP and top 40 down-regulated genes in rapamycin (RAPA+) treated parasite over a 48-hour IDC cycle of wild-type parasites (Subudhi et al., 2020). PfAP2-MRP shows two peaks of expression during IDC, the first peak at ˜16 h.p.i. and the second peak at ˜40 h.p.i. The peak of expression of the majority of known egress and invasion associated genes shown in the line graph either coincides with the second peak of expression of PfAP2-MRP or is just after that. FIG. 6B is a schematic showing the protein structure of PfAP2-MRP that contains a nuclear localization signal (NLS) and a single AP2 DNA binding domain. Both the NLS and AP2 DNA binding domain are encoded from the exon 2 sequence of the gene. FIG. 6C is a schematic representation of the phylogenetic distribution of AP2 genes in alveolate genomes. Dark boxes denote the presence of AP2 genes detected through reciprocal best BLAST hits to P. falciparum protein sequences. Light boxes indicate that no specific gene is detected but that the organism genomes encode an ortholog to a detected AP2 gene. The presence of AP2 protein domains are shown as circles. FIGS. 6D and 6E are electron micrographs of iRBCs from PfAP2-MRP control (FIG. 6D) and PfAP2-MRP (RAPA+) (FIG. 6E) samples. Bar represent lum (FIGS. 6D and 6E) and 100 nm in the inserts. Low power image through a mature schzont showing a number of fully formed merozoites each with a nucleus and electron dense rhoptires (R) in the apical cytoplasm located around a residual body with a food vacuole (FD) (FIG. 6D). A—apical polar ring; N—nucleus. Insert. Longitudinal section through a merozoite showing the apical ring (A), underlying rhoptires (R) and a posterior nucleus (N) enclosed by the pellicle (P). Low power image of early segmenter illustrating the formation of the apical ends (arrowheads) of a number merozoites with underlying nuclei (N) and rhoptries (R) at the surface of the schizont (FIG. 6E). Insert. Detail of the enclosed area in e illustrating the periphery of the schizont showing the limited growth of the apical IMC (arrowheads) forming the merozoite pellicle (P). Note the underlying nucleus (N) and rhoptries (R) of the daughter anlagen. FIG. 6F is a bar plot showing different developmental stages of Compound 2 treated (49 h.p.i) and RAPA treated parasites (49 h.p.i.); Visual inspection of 50 random iRBCs from each group was carried out and cells were demarcated as either mature schizont, segmenter or dead. FIG. 6G shows RNA-seq data from different IDC time-points mapped to the pfap2-mrp locus; there is a drastic reduction in RNA-seq reads mapping to the second exon 16 hours after the addition of rapamycin i.e. at 20 h.p.i. FIG. 6H is a schematic showing rapamycin treatment schedule to disrupt either first or second peak of pfap2-mrp expression

FIGS. 7A and 7B are volcano plots showing significantly differentially expressed genes in rapamycin-treated compared to control parasites at 16 h.p.i. (FIG. 7A) and 40 h.p.i. (FIG. 7B). FIG. 7C shows non-differentially expressed genes in treated [RAPA (+)] compared to control [RAPA (−)] parasites at 40 h.p.i., which had been reported to express at least 4-fold higher in schizont-stage parasites (>35 h.p.i.) compared to early-stage parasites (<35 h.p.i.)⁵. FIG. 7D shows expression of members of the gene families: rifin, stevor and Pfinc-2tm at 16 h.p.i. that encode antigenically variant proteins. FIG. 7E shows gene-ontology (GO) enrichment analysis of all genes down-regulated in rapamycin-treated compared with control parasites at 40 h.p.i. Shown is the number of genes down-regulated in treated parasites out of the total number of genes assigned to that specific GO term. FIG. 7F shows heatmap of the expression of all known and putative P. falciparum kinases downregulated following rapamycin treatment compared to controls, at 40 h.p.i. FIG. 7G shows violin plots of all surfin, rifin, stevor and pfmc-2tm expression per cell in treated [RAPA (+)] and control [RAPA (−)] parasites at 16 h.p.i. FIG. 7H shows FACS gating strategy for surface PfEMP1 expression detected by IgG binding from pooled serum of malaria-infected individuals. Cells were incubated either without (HS−) or with (HS+) pooled serum from malaria-infected individuals. The percentage of Human IgG+iRBCs is the mean of three biological replicates. A total of 7,500 Sybr green positive events (iRBCs) per sample were analyzed.

FIG. 8A shows gene ontology enrichment analysis of up-regulated genes in Δpfap2-mrp parasites at 40 h.p.i. FIG. 8B shows differential expression in Δpfap2-mrp parasites at 40 h.p.i. and 16 h.p.i. of genes that are known or putative early gametocyte markers. FIG. 8C shows differential expression of selected genes in RAPA-treated and control parasites measured using qRT-PCR, to validate RNA-seq data.

FIG. 9A shows enrichment of PfAP2-MRP bound reads (from replicate 1) around the ±5 kb region of peak summits, from 16 h.p.i. (left panel) and 40 h.p.i. (right panel) parasite samples. FIG. 9B shows the position of ChIP-seq peak summits (common between two biological replicates) relative to the predicted ATG translational start codon, in 16 h.p.i. (red) and 40 h.p.i. (blue) parasites. FIG. 9C shows three independent PfAP2-MRP ChIP experiments followed by qPCR, were performed to validate ChIP-seq data, using selected PfAP2-MRP-bound promoter regions of genes from samples at 40 h.p.i. The bar-plot shows percent input (% Input) enrichment of PfAP2-MRP on target genes (mean±SD of three independent experiments). IgG was used as the mock-treated control. P-values were calculated using a two-tailed t-test. FIG. 9D shows input subtracted ChIP peaks of PfAP2-MRP in chromosome 4 and 7 as representatives in both 16 and 40 h.p.i. stages. Also, zoomed in PfAP2-MRP bound central chromosomal and sub-telomeric heterochromatin regions are shown. X-axis shows the genomic position and numbers on the right show the enrichment score. FIG. 9E is a schematic diagram of the chromosomal position of var genes with promoters bound by PfAP2-MRP at either 16 h.p.i. (green), 40 h.p.i. (red) or at both stages (black).

FIG. 10 is a series of ChIP tracks showing the occupancy of PfAP2-MRP in the promoter region of apiap2 genes. ChIP tracks show input subtracted PfAP2-MRP-ChIP from 16 and 40 h.p.i. parasites. Arrow marks show the direction of gene transcription X-axis shows the genomic position, and numbers on the right show the enrichment score.

FIG. 11A shows the occupancy of PfAP2-MRP in the promoter region of pfap2-I at 16 and 40 h.p.i. FIG. 11B shows occupancy of PfAP2-MRP and PfAP2-I in the promoter region of pfap2-mrp at 16 and 40 h.p.i. FIG. 11C is a schematic showing probable gene regulatory network between PfAP2-MRP and PfAP2-I. Orange arrows indicate the binding of protein to its gene promoter. FIG. 11D shows comparison of genes with promoters bound by PfAP2-MRP (red), PfAP2-I (green) and PfAP2-G (light blue) at 40 h.p.i. FIG. 11E shows input-subtracted ChIP-seq read coverage for exemplar genes with promoters that are either bound by all three AP2 proteins (left panel), uniquely by PfAP2-MRP, by both PfAP2-MRP and PfAP2-I, or by both PfAP2-MRP and PfAP2-G (right panel). Arrows show the direction of gene transcription. X-axes show the chromosomal position, and the numbers on the side show the peak enrichment score. X-axis shows the genomic position and numbers on the right show the enrichment score.

FIG. 12 is a series of schematics showing that deletion of pfap2-mrp before the first peak of expression at 16 h.p.i. affects parasite development beyond late trophozoite/early schizont stages. Deletion of pfap2-mrp well before 40 h.p.i. but after the first peak of expression at 16 h.p.i. affects merozoite development and blocks parasite egress from infected RBCs. At this late stage of intraerythrocytic development, the second peak of pfap2-mrp expression activates many genes associated with invasion, egress, antigenic variation, host cell remodeling and protein phosphorylation, either directly by binding to their promoter or indirectly through other downstream ApiAP2 transcription factors and regulators. PfAP2-MRP is a direct repressor of var genes and an indirect repressor of many gametocytogenesis-associated marker genes. Deletion of pfap2-mrp derepresses expression of most of var genes leading to the displayed of the corresponding PfEMP1 on the iRBC surface. PfAP2-MRP acts as a direct activator of many other genes such as rifins, stevors, and pfmc-2tms coding for antigenically variant proteins, and binds to the promoter of 14 other ApiAP2s (50% of all P. falciparum ApiAP2 genes), suggesting that it is an upstream regulator of gene expression cascades during the IDC. PfAP2-MRP associates with many known and putative histone modifiers and chromatin remodelers that probably participate in PfAP2-MRP associated gene regulation. Altogether, PfAP2-MRP regulates the expression of most known pathogenic factors (associated with antigenic variation and parasite growth) in P. falciparum suggesting it is a master regulator of malaria pathogenesis

FIG. 13 is a series of ChIP tracks showing occupancy of PfAP2-MRP in the promoter region of different invasion associated genes. The ChIP tracks show two replicates with input subtracted from the PfAP2-MRP-ChIP data. Arrows indicate direction of transcription. X-axis shows the chromosomal position, and numbers on the right, show the enrichment score.

### DETAILED DESCRIPTION OF THE INVENTION

### I. Definitions

As used herein, “isolated,” “isolating,” “purified,” “purifying,” “enriched,” and “enriching,” when used with respect to a compound of interest, indicates that the compound of interest at some point in time were separated, enriched, sorted, etc., from or with respect to other material to yield a higher proportion of the compound of interest compared to the other materials, for example, cellular material, contaminates, or active agents such as enzymes, proteins, detergent, cations, anions, or other compounds. “Highly purified,” “highly enriched,” and “highly isolated,” when used with respect to a compound of interest, indicates that the compound of interest is at least about 70%, about 75%, about 80%, about 85%, about 90% or more, about 95%, about 99% or 99.9% or more purified or isolated from other materials such as cellular materials, contaminates, or active agents such as enzymes, proteins, detergent, cations or anions. “Substantially isolated,” “substantially purified,” and “substantially enriched,” when used with respect to a compound of interest, indicates that the compound of interest is at least about 70%, about 75%, or about 80%, more usually at least 85% or 90%, and sometimes at least 95% or more, for example, 95%, 96%, and up to 100% purified or isolated from other materials, such as cellular materials, contaminates, or active agents such as enzymes, proteins, detergent, cations or anions.

The terms “nucleic acid molecule,” “nucleic acid sequence,” “nucleic acid fragment,” “oligonucleotide” and “polynucleotide” are used interchangeably and are intended to include, but not limited to, a polymeric form of nucleotides that can have various lengths, either deoxyribonucleotides (DNA) or ribonucleotides (RNA), or analogs or modified nucleotides thereof, including, but not limited to locked nucleic acids (LNA) and peptide nucleic acids (PNA). An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides.

In some cases, nucleotide sequences are provided using character representations recommended by the International Union of Pure and Applied Chemistry (IUPAC) or a subset thereof. IUPAC nucleotide codes used herein include, A=Adenine, C=Cytosine,G=Guanine, T=Thymine, U=Uracil, R=A or G, Y=C or T, S=G or C, W=A or T, K=G or T, M=A or C, B=C or G or T, D=A or G or T, H=A or C or T, V=A or C or G, N=any base, “.” or “-”=gap. In some embodiments the set of characters is (A, C, G, T, U) for adenosine, cytidine, guanosine, thymidine, and uridine respectively. In some embodiments the set of characters is (A, C, G, T, U, I, X, Ψ) for adenosine, cytidine, guanosine, thymidine, uridine, inosine, uridine, xanthosine, pseudouridine respectively. In some embodiments the set of characters is (A, C, G, T, U, I, X, Ψ, R, Y, N) for adenosine, cytidine, guanosine, thymidine, uridine, inosine, uridine, xanthosine, pseudouridine, unspecified purine, unspecified pyrimidine, and unspecified nucleotide respectively.

As used herein, the term “immune cell” refers to cells of the innate and acquired immune system including neutrophils, eosinophils, basophils, monocytes, macrophages, dendritic cells, lymphocytes including B cells, T cells, and natural killer cells.

The term “antigen” as used herein is defined as a molecule capable of being recognized or bound by an antibody, B-cell receptor or T-cell receptor. An “immunogen” is an antigen that is additionally capable of provoking an immune response against itself (e.g., upon administration to a mammal, optionally in conjunction with an adjuvant). This immune response can involve either antibody production, or the activation of specific immunologically-competent cells, or both. Any macromolecule, including virtually all proteins or peptides as well as lipids and oligo- and polysaccharides, can serve as an antigen or immunogen. Furthermore, antigens/immunogens can be derived from recombinant or genomic DNA. Any DNA that includes a nucleotide sequences or a partial nucleotide sequence encoding a protein or peptide that elicits an immune response therefore encodes an “immunogen” as that term is used herein. An antigen/immunogen need not be encoded solely by a full-length nucleotide sequence of a gene. An antigen/immunogen need not be encoded by a “gene” at all. An antigen/immunogen can be generated, synthesized, or can be derived from a biological sample. Such a biological sample can include, but is not limited to a tissue sample, a tumor sample, a cell or a biological fluid.

The term “small molecule,” as used herein, generally refers to an organic molecule that is less than about 2,000 g/mol in molecular weight, less than about 1,500 g/mol, less than about 1,000 g/mol, less than about 800 g/mol, or less than about 500 g/mol. Small molecules are non-polymeric and/or non-oligomeric.

As used herein, the term “carrier” refers to an organic or inorganic ingredient, natural or synthetic, with which the active ingredient is combined to facilitate the application.

As used herein, the term “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

As used herein, the term “pharmaceutically acceptable carrier” means one or more compatible solid or liquid fillers, dilutants or encapsulating substances which are suitable for administration to a human or other vertebrate animal.

“Treatment” or “treating” means to administer a composition to a subject or a system with an undesired condition (e.g., an infectious disease, cancer). The condition can include one or more symptoms of a disease, pathological state, or disorder. Treatment includes medical management of a subject with the intent to cure, ameliorate, stabilize, or prevent a disease, pathological condition, or disorder. This includes active treatment, that is, treatment directed specifically toward the improvement of a disease, pathological state, or disorder, and also includes causal treatment, that is, treatment directed toward removal of the cause of the associated disease, pathological state, or disorder. In addition, this term includes palliative treatment, that is, treatment designed for the relief of symptoms rather than the curing of the disease, pathological state, or disorder; preventative treatment, that is, treatment directed to minimizing or partially or completely inhibiting the development of the associated disease, pathological state, or disorder; and supportive treatment, that is, treatment employed to supplement another specific therapy directed toward the improvement of the associated disease, pathological state, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

As used herein, the terms “effective amount” or “therapeutically effective amount” are used interchangeably and mean a quantity sufficient to alleviate or ameliorate one or more symptoms of a disorder, disease, or condition being treated, to induce or enhance an immune response, or to otherwise provide a desired pharmacologic and/or physiologic effect. Such amelioration only requires a reduction or alteration, not necessarily elimination. The precise quantity will vary according to a variety of factors such as subject-dependent variables (e.g., age, immune system health, weight, etc.), the disease or disorder being treated, the disease stage, as well as the route of administration, and the pharmacokinetics and pharmacodynamics of the agent being administered.

As used herein, the term “reduce”, “inhibit”, “alleviate” or “decrease” are used relative to a control. One of skill in the art would readily identify the appropriate control to use for each experiment.

As used herein, the terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. Thus, adult and newborn subjects, whether male or female, are intended to be covered. A subject can include a control subject or a test subject.

Recitation of ranges of values herein are merely intended to serve as a shorthand method of referring individually to each separate value falling within the range, unless otherwise indicated herein, and each separate value is incorporated into the specification as if it were individually recited herein.

### II. Compositions

### A. Parasitic Proteins Sequence

The rapid and successive events of parasite egress and invasion take place over a relatively short duration (˜15 mins) and involve a large number of proteins, including several that participate in both the processes (13, 32). This indicates that these proteins need to be available within a short time-window just before egress. Based on analogies with other developmentally-regulated Plasmodium genes, it is likely that the proteins are co-expressed, and their expression is probably driven by a single transcription factor (or functionally related transcription factors). In Plasmodium, a single Apicomplexan-specific ApiAP2 family of DNA binding proteins has emerged as the major transcriptional regulators driving various developmental and differentiation processes (33-36). A total of 27 ApiAP2 transcription factors have been identified in P. falciparum so far, all of which are characterized by the presence of 1-3 APITELA2/ethylene response factor DNA binding domains (33, 37). Santos et al. have identified an ApiAP2 known as PfAP2-I that interacts with a specific motif upstream of the transcriptional start site of several rhoptry-associated and MSP-encoding genes (38). However, there are still many secretory organelle, parasite surface or IMC-associated invasion genes (including those encoding the EBLs and Rhs), as well as those encoding genes involved in egress, the transcriptional regulators of which have not yet been identified.

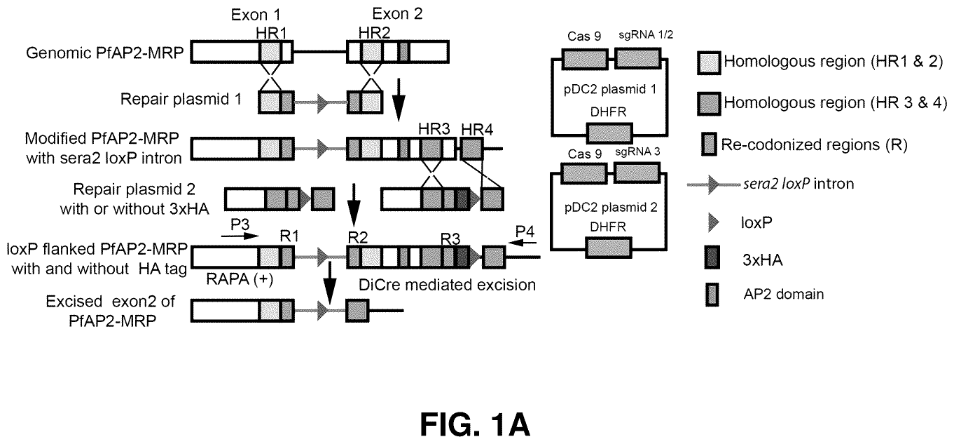

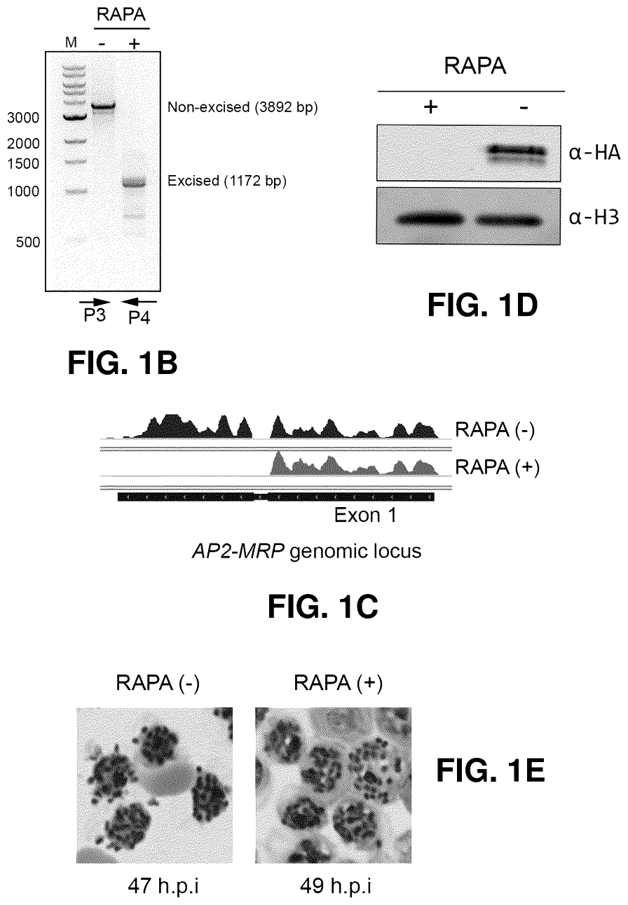

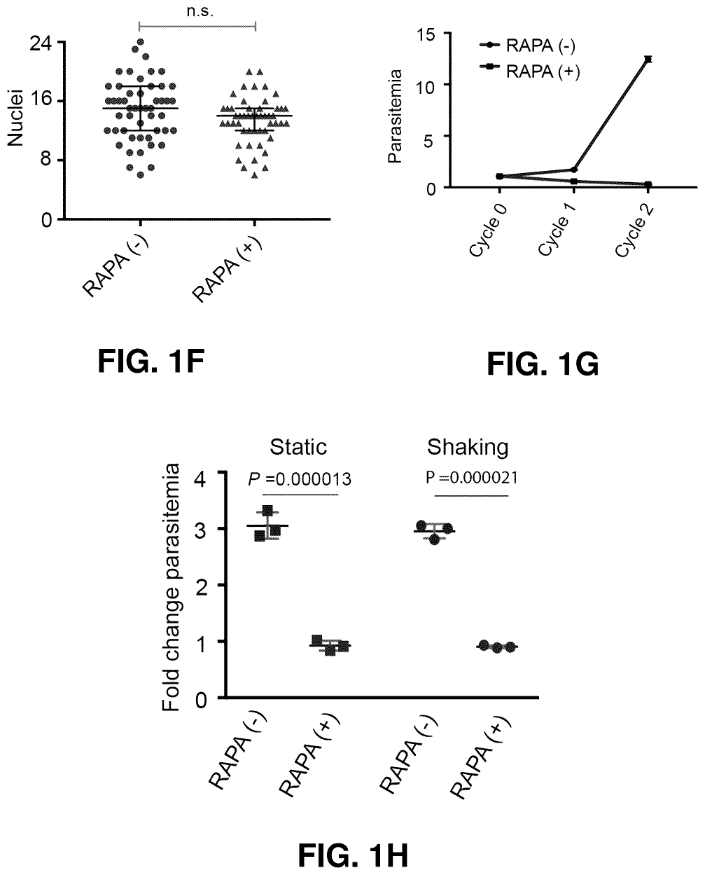

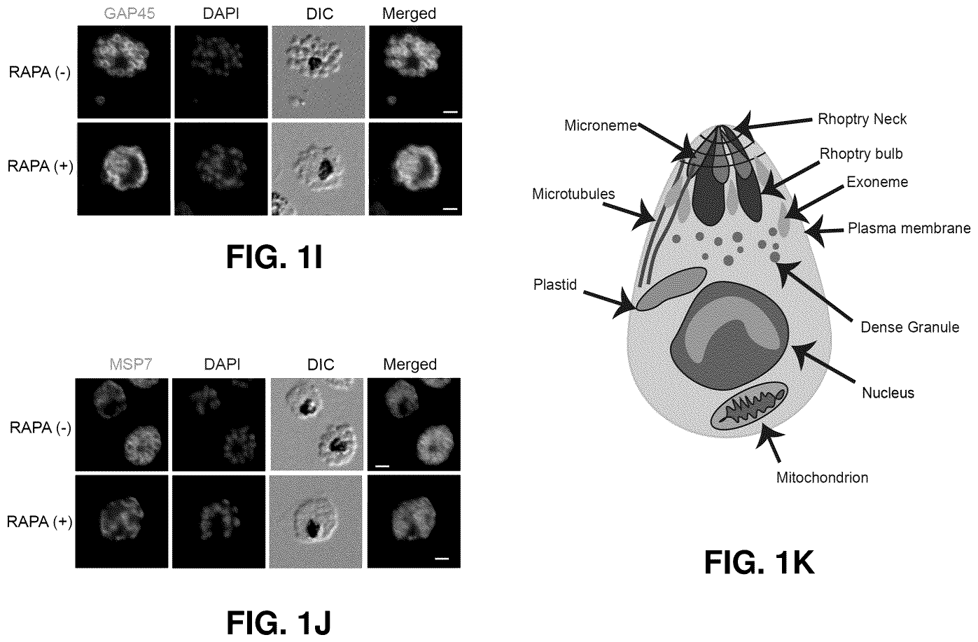

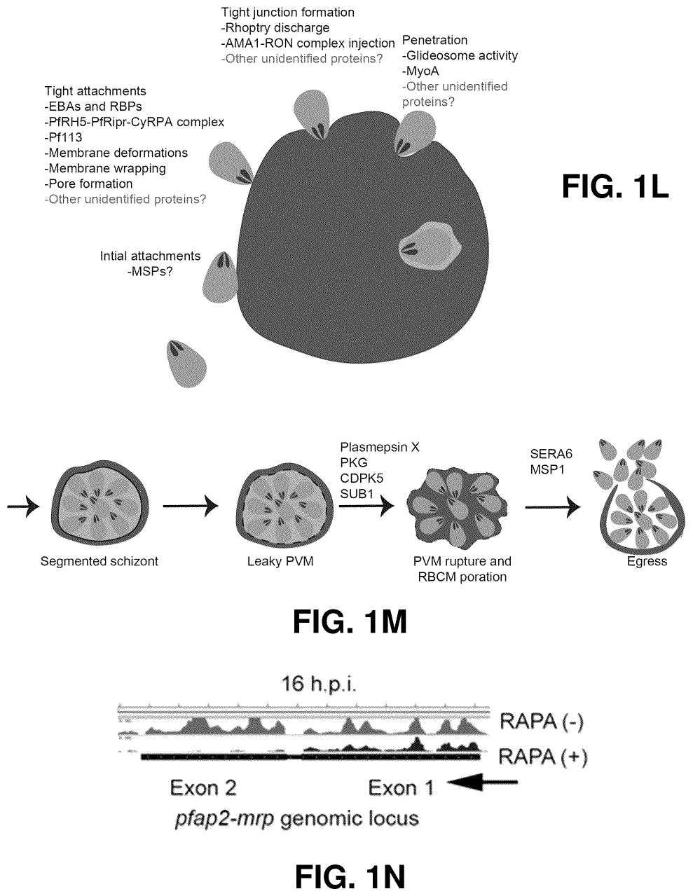

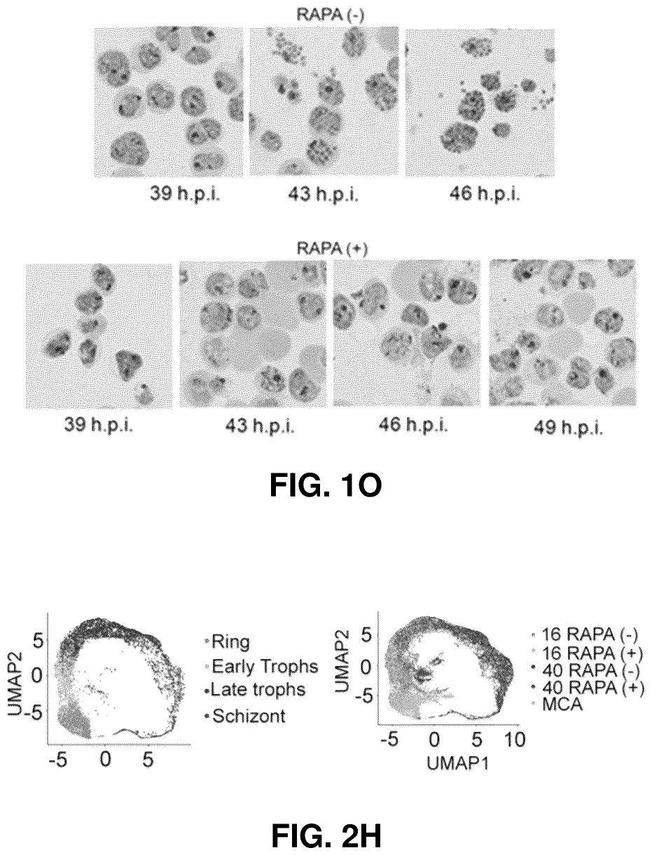

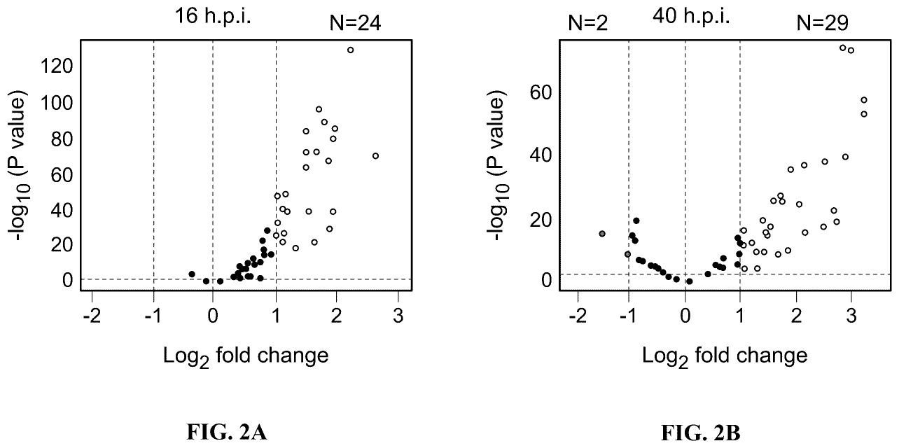

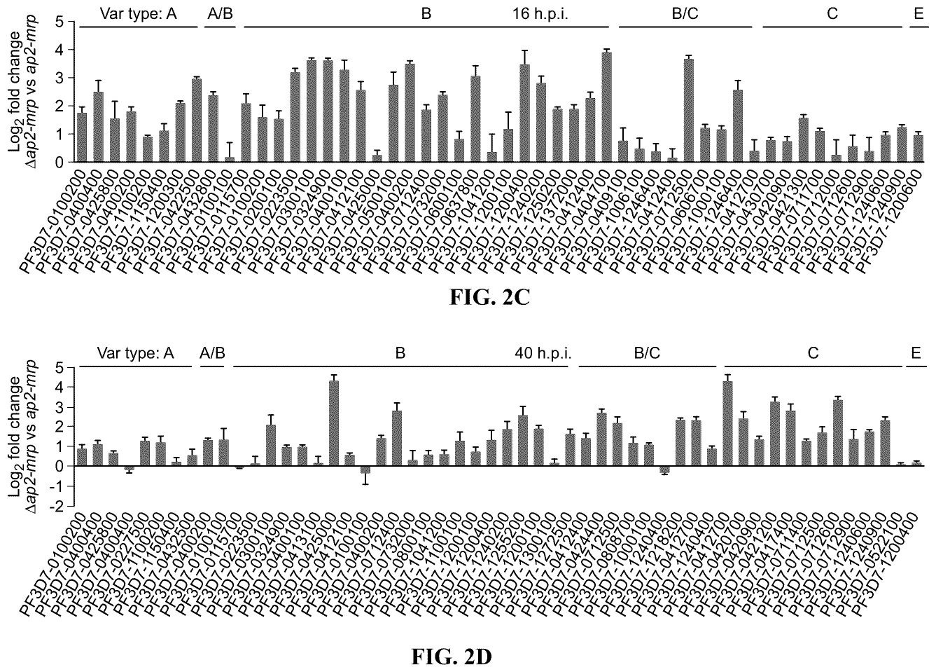

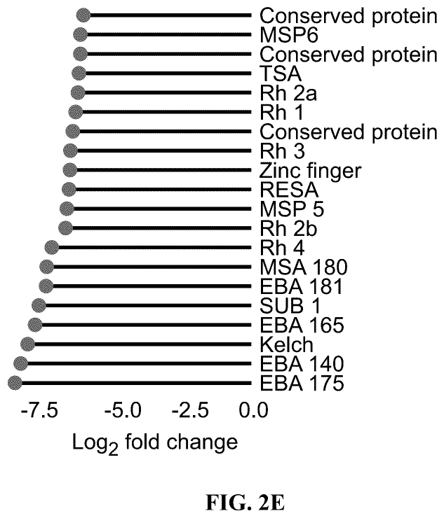

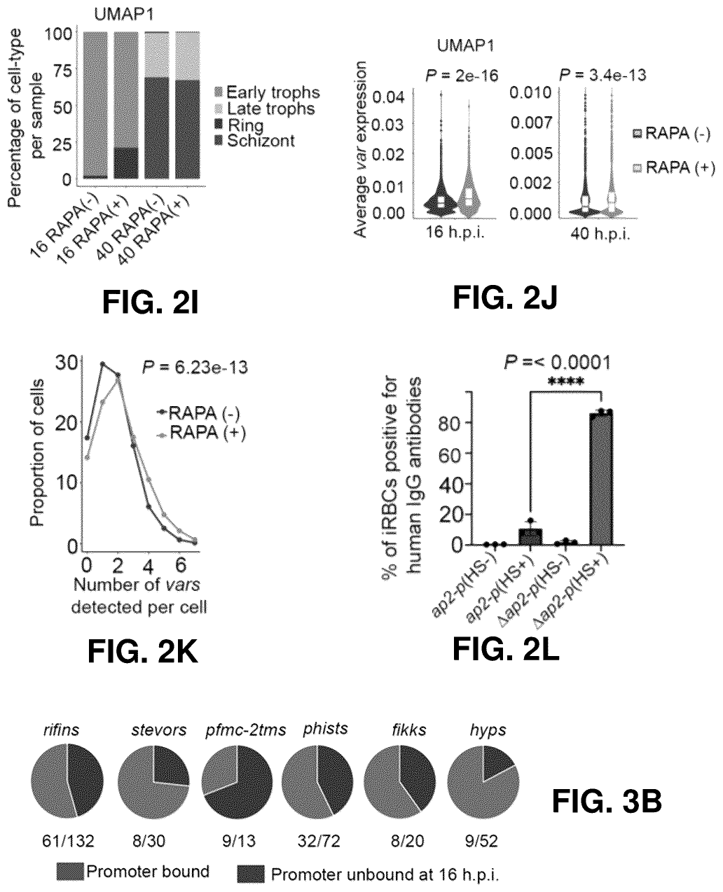

The Examples below evidence the identification of an essential P. falciparum transcription factor named PfApiAP2-MRP (master regulator of pathogenesis), that selectively regulates expression of the majority of known parasite genes associated with egress and invasion. Conditional disruption of PfApiAP2-MRP showed that ΔPfApiAP2-MRP parasites form morphologically normal mature segmented schizonts at the end of the erythrocytic cycle of gene disruption (referred to as ‘cycle 0’) (FIG. 1E) indicating that PfApiAP2-MRP is not required for intracellular parasite development. However, PfApiAP2-MRP parasites failed to egress, and in subsequent asexual replication cycles there was a dramatic reduction in parasite replication compared to control cultures (FIGS. 1G and 1H) indicating an important role for PfApiAP2-MRP in parasite growth. Conditional disruption of PfApiAP2-MRP at 40 hours post-invasion (40 hpi), the point of peak expression of PfApiAP2-MRP during the intraerythrocytic developmental cycle, revealed down-regulation of as many as 763 genes (Padj<0.05, log 2 fold change <−1) in ΔPfApiAP2-MRP parasites (FIG. 2B). Intriguingly, examination of the down-regulated genes showed that 14 of the 20 most significantly down-regulated genes were either established egress-related (e.g. SUB1) or invasion-related genes (e.g., EBA-175, 140 and 181; Rhl, 2a, 2b and 4) (e.g., FIG. 2G). In addition, strong down regulation of many hypothetical protein encoding genes whose function is not elucidated yet (e.g., FIG. 2H) was observed. Down-regulated genes encoding proteins associated with merozoite invasion in ΔPfApiAP2-MRP parasites include almost all known microneme proteins, rhoptry proteins, and merozoite peripheral proteins, as well as many IMC and glideosome proteins (FIG. 2E). Downregulation and/or processing defects were also confirmed in a handful of proteins (AMA1, PTRAMP, SUB1, ARO, and MyoA) in the ΔPfApiAP2-MRP parasites that are in line with the RNA-seq data. These exciting results perfectly complement the phenotypic observation that ΔPfApiAP2-MRP parasites fail to egress, strongly supporting the conclusion that PfApiAP2-MRP plays an essential role in parasite egress and invasion by regulating the expression of the majority of genes required for these processes.

The list of down-regulated genes in ΔPfApiAP2-MRP parasites also included many genes encoding conserved Plasmodium proteins of unknown function and with no recognizable protein domains (hypothetical proteins). For example, out of the top 20 down-regulated genes, 13 genes are known to be associated with parasite invasion, and egress whilst 3 others encode conserved genes of unknown function (FIG. 2G). An extensively validated principle termed ‘guilt by association’ has shown that functionally associated proteins tend to co-express under the control of common transcriptional regulator(s) (39). Using this principle, the function of hypothetical genes can be identified based on their degree of co-expression with other genes with known functions in various developmental and perturbed conditions. Based on this approach, Hu et al., (2010) (40) annotated the function of 1,376 hypothetical genes in P. falciparum.

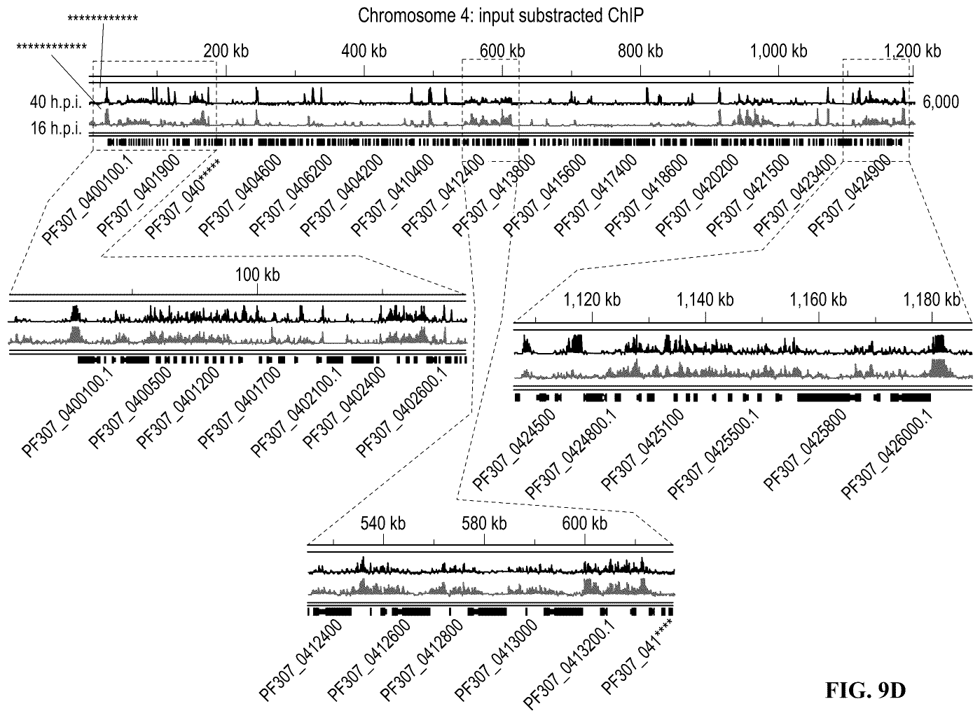

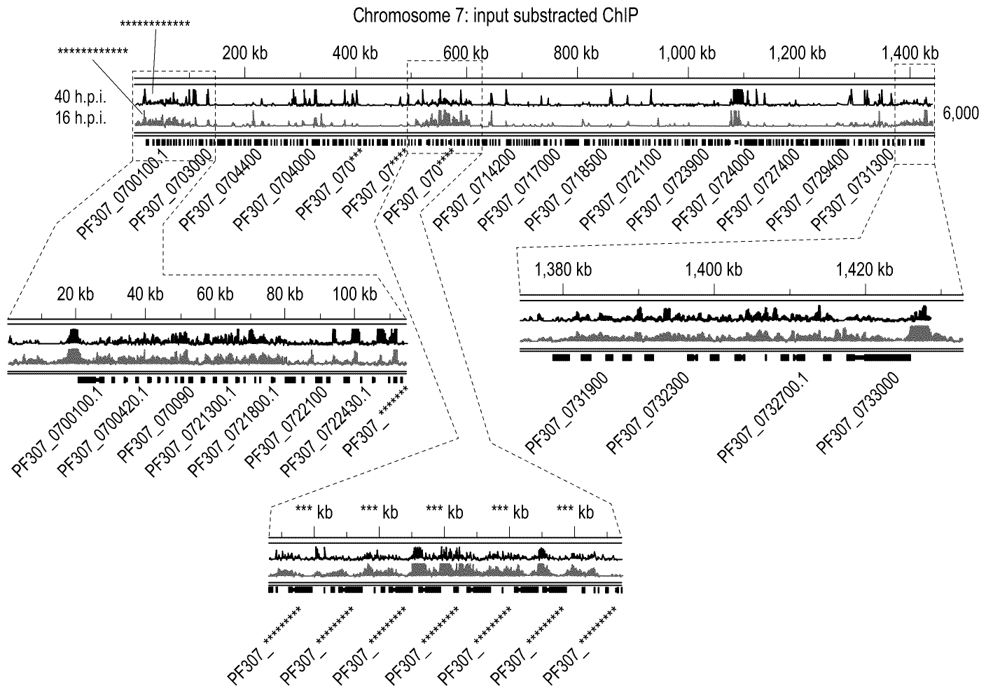

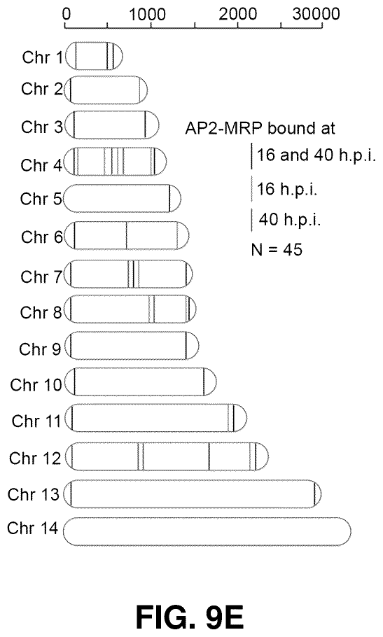

In order to test whether the PfApiAP2-MRP-dependent hypothetical genes co-express with other known egress and invasion-associated genes under a wide range of conditions and developmental stages, a co-expression-based network analysis was performed using the STRING database (41). The top down-regulated genes (log 2-fold change≤−4 to −9, Padj ≤−0.05, N=100 genes) were found to be significantly more interconnected to each other (protein-protein enrichment p<1.0e-16) than expected in the P. falciparum co-expression network (FIG. 9E). Remarkably, significantly more interactions than expected by chance were also observed between all the down-regulated genes. This observation indicates that the down-regulated hypothetical genes that co-express with other known genes associated with invasion and egress are likely candidates for previously unidentified members of the same crucial pathogenic processes. Based on the above analysis and the ‘guilt by association’ principle, it is believed that the majority of the top down-regulated conserved genes of unknown function in ΔPfApiAP2-MRP parasites likely represent previously unidentified components of the egress and invasion pathways. Notably, the two top-most significantly regulated genes encoding hypothetical proteins (Pf3D_71014100, Pf3D7_0210600) in the ΔPfApiAP2-MRP parasites have very recently been experimentally found to associate with the P. falciparum invasion pathway; these genes encode PfMSA18042 and the cytosolically exposed rhoptry leaflet interacting protein 1 (PfCERLI1) (43), respectively. Furthermore, conditional disruption of Pf3D7_1014100 (encoding PfMSA180) resulted in a complete block in parasite egress, with no impact on intracellular parasite maturation. Collectively, these observations evidence that many of the other PfApiAP2-MRP I-dependent hypothetical protein-encoding genes are involved in invasion and/or egress.

Thus provided are proteins of previously unknown function, most particularly the top 14 proteins, shown in the Examples below to be encoded by genes that showed strong down-regulation in ΔPfApiAP2-MRP parasites (log 2-fold change of −4 to −9) and their orthologs across all the human infecting malaria species such as P. vivax, P. malariae, P. ovale curtisi, P. ovale wallikeri, P. knowlesi, P. cynomolgi, P. simium (Table 1; FIG. 2H; Table 2), and compositions and methods of use thereof for prophylactic and therapeutic interventions including, but not limited to vaccines and/or inhibition.

These genes (and for brevity, the proteins encoded by them) are referred to as egress and invasion-associated genes or “EIAGs,” which encompasses both the 14 P. falciparum of Table 1, the orthologs of Table 2, and other orthologs in other species. The P. falciparum invasion machinery includes numerous proteins with varying levels of functional redundancy, in-part because P. falciparum employs several alternative invasion pathways that use different parasite-ligand and host-receptor pairs, possibly in order to accommodate phenotypic diversity in human RBCs (13). Some of the EIAGs are either genus-specific or P. falciparum-specific and some have orthologs in other Apicomplexan parasites. All 14 EIAGs are densely connected with other known invasion and egress associated genes and their expression peaks at the same time as PfApiAP2-MRP during the intra-erythrocytic developmental cycle (IDC), indicating a shared biological function. Furthermore, all 14 EIAGs were previously predicted to be involved in invasion by Hu et al., 2010 (40) based on their co-expression pattern in a large-scale growth perturbation study in P. falciparum.

TABLE 1
List of hypothetical protein encoding genes under investigation.

| EIAG | Corresponding P. falciparum Gene ID |
|---|---|
| EIAG-1 | PF3D7_0726400 |
| EIAG-2 | PF3D7_1413700 |
| EIAG-3 | PF3D7_0308300 |
| EIAG-4 | PF3D7_0408000 |
| EIAG-6 | PF3D7_0903600 |
| EIAG-6 | PF3D7_1331500 |
| EIAG-7 | PF3D7_1307500 |
| EIAG-8 | PF3D7_1146200 |
| EIAG-9 | PF3D7_0625400 |
| EIAG-10 | PF3D7_0911100 |
| EIAG-11 | PF3D7_0607400 |
| EIAG-12 | PF3D7_0805300 |
| EIAG-13 | PF3D7_1026600 |
| EIAG-14 | PF3D7_1411000 |

Protein sequences are provided below.

PF3D7_0625400 | Plasmodium falciparum 3D7 |
conserved Plasmodium protein, unknown
function | protein | length = 412
(SEQ ID NO: 1)
MKFFVLFLLVTLNLFHISLEHNVEEEKCDYMNIFTKPLFYNCYVF
KDHGNKIQQGCCKYYKAVRKGYERKSEELYKLSEEEYNKQYGNIK
TDLNKNDLYSINDIIKNSISEKDMDEIKHLFPVIGEKLLNMNGQD
GNINIKRSITDIDDLLVEFNNKHFNLKDIQVKILNGSTKNYDAYF
NHIKADQNFIKKKEEILQKSPFYNKTCFKSLGGKNCNNGNFNHNN
NNKAHEDDDNDDEDLNYELYEQYKEDDNLNSPSNNMNNIPNGNVN
NNDNTINNIHNNSTNNDTYNNSVTYNHPQYTELEEDNINNKSSYY
SFTQNFSIVNLFSQNPRKVMQKYLEFKHHLSSPSKEKMNGFLDRE
YYNTTKKLNEVLEDDLKKTGETIDQKGKKGFVSSFFDDLMSLIYF
PKKNVEL;
>PF3D7_0725400 | Plasmodium falciparum 3D7 |
conserved Plasmodium protein, unknown
function | protein | length = 109
(SEQ ID NO: 2)
MCFLKYFIFSLIWIFLYNEKAKDNSFLMNHNKREYNVPNGRILRL
DTPEPWGDSETYEVVTKDKHGNLITTVFNKNAEALYFYINKKKPK
EKNSSKPKNSKMRKKGQNN;
>PF3D7_0911100 | Plasmodium falciparum 3D7 |
START domain-containing protein,
putative | protein | length = 1165
(SEQ ID NO: 3)
MSCVLKKITVSEENICCYTGEEEILEKEKKKNARDLLVESKKEKN
KKIEQKKQLPLREKLPTYFSQNSSNSLKNSNFDKKQFCESGTKYL
IKYKIYNLIKNQFYQVDVHHASVSPSLDKKKLNSTKNIIREKNKQ
TNKKKKKLPKIPEKKKKDFLINIATNFRKNKPKKNSENSNKKKKK
KNYTNQQAKKDKSNNNIINNSNSNINNNSNSNSNSNSNNNNNYYY
YYYNNKNKNKNSNNNNNNNNNLKNKNKNNKRNRKGKKNIKKKIYI
EKNSLTLPKTNRYCNYKYEEYYDISDEKMEATNYDETYCYDEELK
NDSKTTIYDEGVSKKNIYNCDVNHIFDNFCNYFNCVDKKESMNDD
TANSEITLNSNNIKTQDIIIDNNEKSFLYEKNRQQNINVYTQTSN
KKSRWLCSTADDSHNEYLTEYEKKIDSYDNIDNNVHKDYSNIHKN
VTIANNNNITLNYDTNDFSSYTTQPIQLFNDQENNTYDMGTNIVR
KGKSLYEETRKISNALFEMYSTNVNKDNMNNNMNGNNNNNNNNNE
NVDKLCNKNQLEAPTPSLFTTYINQSHNYHSIVDKNIVLVQIKNF
SQKIDEYISDEKIFRAQKLIDHVKKYIEFYINYYKKYNDKEVVEK
LELYHEMHCMHKNIKYNINNLKVNIIMHFLNFFHLNDMYTILNDY
TYYFSVDMVNSITSESITNSNENTNSINHSDINNMKTKNSIHSYA
PSELGGLFNSSIKCYSIDESLNSSKMYLTPTLGNNNNNNINVSNN
EAENIENVHKSDNLNNNNNNNEDDGVNVDSGINNNSKYNTDDSDI
KYEDISSVIVDKIYHQQYSNCNMLNGNKTLINQKSINPDNSANRK
NSNRHMVKIIQKYSKRFKKFRKSKQNSEGWIKENDKYLDLSHRVD
KDNNISVHIRAKLPYEVNRILSILNETELSVNWAPFLTSAKKIKN
LSRASAIITQLYEYPIIGKKESLMYCLGANSLEELGCIILCCKAP
PEFNKDILFYENMCEKININKFGEIIKVKEIPIKFRKTYKEVTFF
DYTLPEPVPKLDRQRAANLCFLLYPMNNGKSTVLELFLHFENEFK
YTPIKMVTFFIKKIVKNMYENIIKSCRNYDLLYSEFLMNNAEFYI
WLDDQIKRYMKGKNDSKLLYSISLESYDEPEHNEELDSKT;
>PF3D7_1026600 | Plasmodium falciparum 3D7 |
conserved Plasmodium protein, unknown
function | protein | length = 1692
(SEQ ID NO: 4)
MKCINAEDKIQQDHEVLYKMFKSLRRHEDDKFINGSIKLKKKSLN
EEINRFKDINDKYIFSNVKKKIKLEINGCIKIKRKGAHEEYYNQL
VKKVLHGSIFIQNKNINNILDYIKYIKYIKLGGKISMVKKKKRTD
KNEIISPLLSKENINKLRYLKTIYDVKKERNQKSPRKSSYNIERK
EILDLSMNRYSHFQSEISNNYNLKNNNFYDSIKLVDNKMGKTKGN
KEKGNKGKEEDKDKDKHKDKDKSKEKEKDINIYKTNGSIKNTLNN
NKCSFVLSPSCNDKIRHVDELEKKMNTFKLNIPSSIINKENKSNN
TNAVENRGSIKYNLIKERQEFMYEPIRNNKEKHNKIHLKGTINYI
NNNNNNKKKNNNNNVHPTKFIFMGKKKKKIIEHEGTDDNYILGNE
EKKKLLYMDQYNNETNKSHETNVLCGNKIYNKCGNKNIFLDHSVF
LNYNKKVPKHISMSCSSKNKDMKKSTYDETMLSMSDVDDIDYFMY
IKEKNAIKYKDYMNKPKTICQDKSSIEKEKSVLRIKKVEDQGDIK
EKKSYMNDLKYKIDKKEPSNKENTSNIRNKKFSSVSRKLSKHFSL
SRSGATFSMSNKKEKKSNKKKRTRFNKENRYMKKYLNKIMNMSIL
RRHFNNVNSSVYITHQKCFDKHEEHTKINEKKCKKGVIKTVDGKE
DMNILNVNNNKEKITTKGNKNDHVGDSKDGNKNDHVGDSKDGNKN
DHVGDSKEDNKNDHVGDSKEDNKNDHVGDSKDNNKNDHVGDSKDN
NKNDHVGDSKDNNKNDHVGDSKEDNTIILSKEKNIKIGIYFEETC
DIKQSNDKNISTTNEIKKMSSPVTNKLLTKKMGFPVTNKLLTKKE
GMLKKFAISKEKMDITKKEGVVLSMKKSFSLKKDAEIHISNDDNV
MDKNKVNSTIKSVQDILSKQLFKSKGNPLLINKKHVPKMDSTNKM
IVDKKISFKSSLFKKDENKKTNYINSEKEEEKNVNMEKFLLNELK
ENYLNDERINKPGLSKEDDVDIKEPVLINEQVDLIKKEDQNICTN
TEQEHEMMQEQKINEDEVNSKGDENTKKTENTINDKETIDIINKK
NSSESVKEEIMEKEMLDEKDKNVPHYLECNNLHNMDENTKEIKVE
KCNDYMTDNITSKNNNMIIKKINMKMLPMVTKKALPKSKALPILL
KKNISIPKEKFTTNKKLSNESIDKTESSNIKMDEHKEEEKQIIKP
SKSIHKIIKPGRHSFFNANKLLNSIEIKIGEHNSKIIEKEGPIKM
FSSSTSIISDGKIDSIHKSDEEEKINDLKEDSNVNIYVKEKGKGK
VFMKKGIPKLMMKFNAFSTKKMGINNLENNKKVNTLRSIKDDKIL
EKTNDTLEEINGTLEKTNGTLEETNGTLEKTNGTLEETSEISNIQ
HEEEVKNEKDATLQDDDDAGHTLKTTKAILMKKIPAKYKMNTVKM
GDIKLPSHMNKFINKFHSVKILSTLKEFPVKKLIKKKDSNEENNS
LEKIKMDKEKINDVDMEKEADEDVMKIEESVMNKKENDETEIEMN
NNNEVDNNEIDNNKVDNNEIDNNKVDNNEIDNNKVDNNEIDNNKV
DNNEIDNNKVDNNEIDNDKVDNISQDKSEISLNKIEEPNLVAEST
TIEKEPIDKIIPRSFSKSLSIPMKVPTIKENINKAKPTFWNKPKG
KMFFLTKKEKSISQAKEILRSLEKSNN;
>PF3D7_1307500 | Plasmodium falciparum 3D7 |
conserved Plasmodium protein, unknown
function | protein | length = 88
(SEQ ID NO: 5)
MFTLISVCSYLFYTHKESLLNLIGLSENRNSRKNRRRKKKKGKLK
QPFPEEKLCLSDSDMRRKKFILRTYSDVNFNVTCTTKKERNSI;
and
>PF3D7_1413700 | Plasmodium falciparum 3D7 |
DET1 domain-containing protein,
putative | protein | length = 1213
(SEQ ID NO: 6)
MCNKYRLHLLNQINPTTNTSSYKQHQLNNDLYKLKKKMEKNKVLF
NKDDKMDILSMDTLKLADEIKTKIIAQQLHTVLVLYLQILKEKKI
EEKNINDDIGQYIENVIISALQLYSLIEKQTKDRKKCYIKKICQL
CNTLSNILIEYIKNIKGIQVQHNSSKINENNQKLLIDSLENSNKL
SKEKSTRSIESSKEKNENVFEGTSGKLNTVSYPCGCVHSQDMKIK
EISLENEFSKGYRLMCLYSAQELNEQINYNKNIINNSFKTFQKKY
CNVKLRDRRELIYKFQKILENKNIQFYDIYKGNVKSNIVSNQVNN
NAQEFHTDLYDGCSYNSNHIIKNVEEKGIRHLINILLHTNKSITT
FEINSQGKIDAIIQFLQKNEKKKDIILLNEYEYFFTNTDDYTQKE
KNKNRRKYRDRENVTCNNRMVKRTYKNVDDNNNNLKEENENIYFY
VGNKKKIVFDIKKKQKIVYEIDEKNSLLTKKKFLKDENNKDILLK
VYDENDWSDENMEFCYITEKKEKEKIVIKKNKHGKKKTVYTNKNN
ELDNNVKNFIFYKKNIKNVKEINFYDDEEVILSLLKQNEERKKLE
DTKDNYDIKNMENTISKDDNTLVCEKNKIPSIWNKAKNKTCTNDR
DMYILEHKYVENKNDEINVESLDTYTNIITNRKEFLLKKHYLDNQ
QDISSFLKIDDYYYFKKNGNDSDTNKFNKYDNDYKMYETNVKGNM
DDTKKCIGNKFDKMRNKKLDNIIAINDDDNNNNNNNYMNNVCLES
YIDLYLEPKKKNEKEKKKKKKNYLINRMNTLISGDGKKNISEEYN
MNDKEKIKIEDFLFNEYLKKVSVRSQSLKKKKEKKHEHEHEHEQE
HEQEHEHEQVHEQEHKHKHEHEHKHKHKHEQEHKHKHEHEHEQVH
EQEHNKIKKIIYDTKIDMKKEEDTQRKIENNHNIISNEKNDILQI
HNIVKKDNKHMEINKCSKNNETHNNLPMSNDQKNNILDKNNKMID
KNLLKKKSSLQMNSFNSDKKLYDNDISNVKEKKIYNERKVSHENS
MENQRYEDDKMGRKYLSEGFSAYVHVALCENTIISKKCIISCNIN
TKMLKIETKEKKFIIDMNKLHVQEIPTTYDNLAIIKLIIKKKNNN
NSNCLLVESKNLGALQHLGDEIGWNYDKTGYSMDHKFNDEKEKKE
RRKTDDEHINNKPQLIDNSTVGFFDKIKLNKFLGKKKTEEKDI.

TABLE 2
Orthologs of 14 P. falciparum EIAGs in other human infecting malaria parasite species.

| | Plasmodium | Plasmodium | Plasmodium | Plasmodium | Plasmodium | Plasmodium |
|---|---|---|---|---|---|---|
| | falciparum | vivax | malariae | ovale [symbol] | knowlesi | cynomolgi |
| EIAGs | 3D7 | P01 | [symbol] G01 | GH01 | strain H | strain M |
| EIAG-1 | [symbol] | [symbol] | [symbol] | [symbol] | [symbol] | [symbol] |
| EIAG2 | [symbol] | [symbol] | [symbol] | [symbol] | [symbol] | [symbol] |
| EIAG-3 | [symbol] | [symbol] | [symbol] | [symbol] | [symbol] | [symbol] |
| EIAG-4 | [symbol] | [symbol] | [symbol] | [symbol] | [symbol] | [symbol] |
| EIAG-5 | [symbol] | [symbol] | [symbol] | [symbol] | [symbol] | [symbol] |
| EIAG-6 | [symbol] | [symbol] | [symbol] | [symbol] | [symbol] | [symbol] |
| EIAG-7 | [symbol] | [symbol] | [symbol] | [symbol] | [symbol] | [symbol] |
| EIAG-8 | [symbol] | [symbol] | [symbol] | [symbol] | [symbol] | [symbol] |
| EIAG-9 | [symbol] | [symbol] | [symbol] | [symbol] | [symbol] | [symbol] |
| EIAG-10 | [symbol] | [symbol] | [symbol] | [symbol] | [symbol] | [symbol] |
| EIAG-11 | [symbol] | [symbol] | [symbol] | [symbol] | [symbol] | [symbol] |
| EIAG-12 | [symbol] | [symbol] | [symbol] | [symbol] | [symbol] | [symbol] |
| EIAG-13 | [symbol] | [symbol] | [symbol] | [symbol] | [symbol] | [symbol] |
| EIAG-14 | [symbol] | [symbol] | [symbol] | [symbol] | [symbol] | [symbol] |

Information about orthologs of EIAGs in other human infecting malaria parasites were collected from OrthoMCL DB (https://orthomcl.org) [symbol]
indicates data missing or illegible when filed

### B. Isolated Nucleic Acids

All of the accession numbers provided herein are specifically incorporated by reference herein in their entireties. For the Gene IDS provided, the genomic, mRNA, and protein sequences provided therein are all expressly provided. Where a nucleic acid sequence is provided in an accession number or elsewhere herein, the encoded amino acid (e.g., polypeptide) sequence is also provided. Where an amino acid (e.g., polypeptide) is provided in an accession number or elsewhere herein, all nucleic acid sequences including but not limited to, gene, cDNA, and mRNA sequences and their complements, and codon variations thereof encoding the amino acid sequence, both as single strands and double strands, and in any form of nucleic acid, including, but not limited to DNA and RNA, and analogs and variations thereof including but limited to modified bases, sugars, and linkages (e.g., peptide nucleic acids (PNA)), are all expressly provided.

Isolated nucleic acid sequences encoding EIAG proteins, polypeptides, fusions fragments and variants thereof, as well as inhibitor nucleic acid, and vectors and other expression constructs encoding the foregoing are also disclosed herein. As used herein, “isolated nucleic acid” refers to a nucleic acid that is separated from other nucleic acid molecules that are present in a mammalian genome, including nucleic acids that normally flank one or both sides of the nucleic acid in a mammalian genome (e.g., nucleic acids that encode non-EIAG proteins). The term “isolated” as used herein with respect to nucleic acids also includes the combination with any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

The nucleic acid sequences encoding EIAG polypeptides include genomic sequences. Also disclosed are mRNA sequence wherein the exons have been deleted. Other nucleic acid sequences encoding EIAG polypeptides, such polypeptides that include the above-identified amino acid sequences and fragments and variants thereof, are also disclosed. Nucleic acids encoding EIAG polypeptides may be optimized for expression in the expression host of choice. Codons may be substituted with alternative codons encoding the same amino acid to account for differences in codon usage between the organism from which the EIAG nucleic acid sequence is derived and the expression host. In this manner, the nucleic acids may be synthesized using expression host-preferred codons.

Nucleic acids can be in sense or antisense orientation, or can be complementary to a reference sequence encoding an EIAG polypeptide. Nucleic acids can be DNA, RNA, or nucleic acid analogs. Nucleic acid analogs can be modified at the base moiety, sugar moiety, or phosphate backbone. Such modification can improve, for example, stability, hybridization, or solubility of the nucleic acid. Common modifications are discussed in more detail below.

Nucleic acids encoding polypeptides can be administered to subjects in need thereof. Nucleic delivery involves introduction of “foreign” nucleic acids into a cell and ultimately, into a live animal. Compositions and methods for delivering nucleic acids to a subject are known in the art (see Understanding Gene Therapy, Lemoine, N. R., ed., BIOS Scientific Publishers, Oxford, 2008).

### 1. Vectors and Host Cells

Vectors encoding EIAG polypeptides, fusion, fragments, and variants and inhibitor nucleic acids thereof are also provided. Nucleic acids, such as those described above, can be inserted into vectors for expression in cells. As used herein, a “vector” is a replicon, such as a plasmid, phage, virus or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. For example, the control sequence can be incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the EIAG polypeptides or fusion polypeptides described herein.

The vectors can be used to express EIAG nucleic acids or nucleic acids inhibitory thereof in cells. An exemplary vector includes, but is not limited to, an adenoviral vector. One approach includes nucleic acid transfer into primary cells in culture followed by autologous transplantation of the ex vivo transformed cells into the host, either systemically or into a particular organ or tissue. Ex vivo methods can include, for example, the steps of harvesting cells from a subject, culturing the cells, transducing them with an expression vector, and maintaining the cells under conditions suitable for expression of the encoded polypeptides. These methods are known in the art of molecular biology. The transduction step can be accomplished by any standard means used for ex vivo gene therapy, including, for example, calcium phosphate, lipofection, electroporation, viral infection, and biolistic gene transfer. Alternatively, liposomes or polymeric microparticles can be used. Cells that have been successfully transduced then can be selected, for example, for expression of the coding sequence or of a drug resistance gene. The cells then can be lethally irradiated (if desired) and injected or implanted into the subject. In one embodiment, expression vectors containing nucleic acids encoding fusion proteins are transfected into cells that are administered to a subject in need thereof.

In vivo nucleic acid therapy can be accomplished by direct transfer of a functionally active DNA into mammalian somatic tissue or organ in vivo. Nucleic acids may also be administered in vivo by viral means. Nucleic acid molecules encoding polypeptides or fusion proteins may be packaged into retrovirus vectors using packaging cell lines that produce replication-defective retroviruses, as is well-known in the art. Other virus vectors may also be used, including recombinant adenoviruses and vaccinia virus, which can be rendered non-replicating. In addition to naked DNA or RNA, or viral vectors, engineered bacteria may be used as vectors.

Nucleic acids may also be delivered by other carriers, including liposomes, polymeric micro- and nanoparticles and polycations such as asialoglycoprotein/polylysine.

In addition to virus- and carrier-mediated gene transfer in vivo, physical means well-known in the art can be used for direct transfer of DNA, including administration of plasmid DNA and particle-bombardment mediated gene transfer.

### 2. Oligonucleotide Composition

The disclosed nucleic acids nucleic acids can be DNA or RNA nucleotides which typically include a heterocyclic base (nucleic acid base), a sugar moiety attached to the heterocyclic base, and a phosphate moiety which esterifies a hydroxyl function of the sugar moiety. The principal naturally-occurring nucleotides include uracil, thymine, cytosine, adenine and guanine as the heterocyclic bases, and ribose or deoxyribose sugar linked by phosphodiester bonds.

In some embodiments, the oligonucleotides are composed of nucleotide analogs that have been chemically modified to improve stability, half-life, or specificity or affinity for a target receptor, relative to a DNA or RNA counterpart. The chemical modifications include chemical modification of nucleobases, sugar moieties, nucleotide linkages, or combinations thereof. As used herein ‘modified nucleotide” or “chemically modified nucleotide” defines a nucleotide that has a chemical modification of one or more of the heterocyclic base, sugar moiety or phosphate moiety constituents. In some embodiments, the charge of the modified nucleotide is reduced compared to DNA or RNA oligonucleotides of the same nucleobase sequence. For example, the oligonucleotide can have low negative charge, no charge, or positive charge.

Typically, nucleoside analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). In some embodiments, the analogs have a substantially uncharged, phosphorus containing backbone.

### 3. Delivery Vehicles

The disclose compounds can be administered and taken up into the cells of a subject with or without the aid of a delivery vehicle. Appropriate delivery vehicles for the disclosed inhibitors are known in the art and can be selected to suit the particular inhibitor. For example, if the compound is a nucleic acid or vector, the delivery vehicle can be a viral vector, for example a commercially available preparation, such as an adenovirus vector (Quantum Biotechnologies, Inc. (Laval, Quebec, Canada). The viral vector delivery can be via a viral system, such as a retroviral vector system which can package a recombinant retroviral genome (see e.g., Pastan et al., (1988) Proc. Natl. Acad. Sci. U.S.A. 85:4486; Miller et al., (1986) Mol. Cell. Biol. 6:2895). The recombinant retrovirus can then be used to infect and thereby deliver to the infected cells nucleic acid encoding the compound inhibitor. The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948 (1994)), adeno-associated viral (AAV) vectors (Goodman et al., Blood 84:1492-1500 (1994)), lentiviral vectors (Naidini et al., Science 272:263-267 (1996)), pseudotyped retroviral vectors (Agrawal et al., Exper. Hematol. 24:738-747 (1996)).

Physical transduction techniques can also be used, such as liposome delivery and receptor-mediated and other endocytosis mechanisms (see, for example, Schwartzenberger et al., Blood 87:472-478 (1996)). For example in some embodiments, the CTPS1 inhibitor is delivered via a liposome. Commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art are well known. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.). This disclosed compositions and methods can be used in conjunction with any of these or other commonly used gene transfer methods.

In some embodiments, the delivery vehicle is incorporated into or encapsulated by a nanoparticle, microparticle, micelle, synthetic lipoprotein particle, or carbon nanotube. For example, the compositions can be incorporated into a vehicle such as polymeric microparticles which provide controlled release of the compound. In some embodiments, release of the drug(s) is controlled by diffusion of the compound out of the microparticles and/or degradation of the polymeric particles by hydrolysis and/or enzymatic degradation. Suitable polymers include ethylcellulose and other natural or synthetic cellulose derivatives. Polymers which are slowly soluble and form a gel in an aqueous environment, such as hydroxypropyl methylcellulose or polyethylene oxide may also be suitable as materials for drug containing microparticles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof.

### C. Live Attenuated Parasites (LAIPs)

The disclosed compositions include Live attenuated (LA)-ΔPFAP2-MRP malaria parasites. Five species of Plasmodium (single-celled parasites) can infect humans and cause illness: Plasmodium falciparum (P. falciparum), Plasmodium malariae (P. malariae), Plasmodium vivax (P. vivax), Plasmodium ovale (P. ovale); and Plasmodium knowlesi (P. knowlesi). Falciparum malaria is potentially life-threatening. Patients with severe falciparum malaria may develop liver and kidney failure, convulsions, and coma. Although occasionally severe, infections with P. vivax and P. ovale generally cause less serious illness, but the parasites can remain dormant in the liver for many months, causing a reappearance of symptoms months or even years later. ΔPFAP2-MRP parasites refer to parasites in which PFAP2-MRP exon2 is deleted. ΔPFAP2-MRP parasites at 40 h.p.i. refers to parasites with disrupted second peak of expression that are collected at 40 h.p.i. and ΔPFAP2-MRP parasites at 16 h.p.i. refers to parasites with disrupted first peak of expression that were collected at 16 h.p.i. LA-ΔPFAP2-MRP malaria parasites can be ΔPFAP2-MRP parasites at 40 h.p.i. ΔPFAP2-MRP parasites at 16 h.p.i. produced as demonstrated in the Examples.

The malaria parasite life cycle involves 2 hosts. During a blood meal, a malaria-infected female Anopheles mosquito inoculates sporozoites into the human host. Sporozoites infect liver cells. There, the sporozoites mature into schizonts. The schizonts rupture and release merozoites. This initial replication in the liver is called the exoerythrocytic cycle. Merozoites infect RBCs. There, the parasite multiplies asexually (called the erythrocytic cycle). The merozoites develop into ring-stage trophozoites. Some then mature into schizonts. The schizonts rupture, releasing merozoites. Some trophozoites differentiate into gametocytes. During a blood meal, an Anopheles mosquito ingests the male (microgametocytes) and female (macrogametocytes) gametocytes, beginning the sporogonic cycle. In the mosquito's stomach, the microgametes penetrate the macrogametes, producing zygotes. The zygotes become motile and elongated, developing into ookinetes. The ookinetes invade the midgut wall of the mosquito where they develop into oocysts. The oocysts grow, rupture, and release sporozoites, which travel to the mosquito's salivary glands. Inoculation of the sporozoites into a new human host perpetuates the malaria life cycle.

The compositions in one embodiment include live attenuated ΔPFAP2-MRP Plasmodium parasites or an orthologue thereof, in P. malariae, P. vivax), Plasmodium ovale P. ovale; and P. knowlesi, which express multiple PfEMP1 proteins on the surface of infected RBCs.

Attenuation can be achieved by chemical treatment of the pathogen, through radiation (Espetin, et al., Science, Vol 334, Issue 6055 pp. 475-48 (2011), or by genetic modification, using methods known to those skilled in the art. Attenuation may result in decreased proliferation, attachment to host cells, or decreased production or strength of toxins.

### Chemical Attenuation

ΔPFAP2-MRP Plasmodiun parasites or an orthologue thereof can be attenuated using methods known in the art. For example, Cyclopropylpyrolloindole analogues, such as centanamycin (CM) and tafuramycin-A (TF-A) have been used to successfully attenuate both sporozoite and asexual blood-stage malaria parasites. For example, Purcell, et al., Infection and Immunity, March 2008, p. 1193-1199, demonstrated effective attenuation of sporozoites using 2 mM centanamycin diluted in Dulbecco modified Eagle medium (DMEM).

### Genetic Attenuation

Genetically attenuated sporozoites (GAS) of ΔPFAP2-MRP Plasmodium parasites, in which genes essential to sporozoite function in parasite strains are deleted, can also be used as live. Methods used in the art include for example, deletion of the uis3, and/or uis4. P36p, genes in Plasmodium berghei. as well as deletions of uis3 and uis4 and simultaneous deletion of the P52 and P36 genes in Plasmodium yoelii. Labaied, et al. Infect. Immun.75:3758-3768. (2007); Jobe, et al, Infect. Dis. 196:599-607.

Methods for providing compositions suitable for routine use in human subjects that comprises live attenuated sporozoites, and live infectious sporozoites are know; the sporozoites must be substantially purified from the source from which they were produced. Pharmaceutical compositions containing substantially purified sporozoites and excipient as well as methods of purifying sporozoites are known in the art (U.S. Patent Publ. No. 2010/018368). This publication is explicitly incorporated herein by reference. Plasmodium-species parasites are grown aseptically in cultures as well as in vivo in Anopheles-species mosquito hosts, most typically Anopheles stephensi hosts. Methods of axenically culturing Plasmodium-species liver stage parasites (US Pub. 2005/0233435) and methods of producing attenuated and non-attenuated Plasmodium-species sporozoites, particularly, methods of growing and attenuating parasites in mosquitoes, and harvesting attenuated and non-attenuated sporozoites are known in the art and have been described (See: U.S. Pat. No. 7,229,627; U.S. Pub. No. 2005/0220822).

### D. Formulations

The disclosed compounds and LAIP can be formulated in a pharmaceutical composition. Pharmaceutical compositions can be for administration by parenteral (intramuscular, intraperitoneal, intravenous (IV) or subcutaneous injection), enteral, transdermal (either passively or using iontophoresis or electroporation), or transmucosal (nasal, pulmonary, vaginal, rectal, or sublingual) routes of administration or using bioerodible inserts and can be formulated in dosage forms appropriate for each route of administration.

The compositions can be administered systemically.

Drugs can be formulated for immediate release, extended release, or modified release. A delayed release dosage form is one that releases a drug (or drugs) at a time other than promptly after administration. An extended release dosage form is one that allows at least a twofold reduction in dosing frequency as compared to that drug presented as a conventional dosage form (e.g. as a solution or prompt drug-releasing, conventional solid dosage form). A modified release dosage form is one for which the drug release characteristics of time course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms.

Formulations are typically prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes, but is not limited to, diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et al. (New York, Marcel Dekker, Inc., 1989), “Remington—The science and practice of pharmacy”, 20th ed., Lippincott Williams & Wilkins, Baltimore, MD, 2000, and “Pharmaceutical dosage forms and drug delivery systems”, 6^{th} Edition, Ansel et. al., (Media, PA: Williams and Wilkins, 1995) which provides information on carriers, materials, equipment and process for preparing tablets and capsules and delayed release dosage forms of tablets, capsules, and granules.

The compound can be administered to a subject with or without the aid of a delivery vehicle. Appropriate delivery vehicles for the compounds are known in the art and can be selected to suit the particular active agent. For example, in some embodiments, the active agent(s) is incorporated into or encapsulated by, or bound to, a nanoparticle, microparticle, micelle, synthetic lipoprotein particle, or carbon nanotube. For example, the compositions can be incorporated into a vehicle such as polymeric particles which provide controlled release of the active agent(s). In some embodiments, release of the drug(s) is controlled by diffusion of the active agent(s) out of the particles and/or degradation of the polymeric particles by hydrolysis and/or enzymatic degradation.

Suitable polymers include ethylcellulose and other natural or synthetic cellulose derivatives. Polymers which are slowly soluble and form a gel in an aqueous environment, such as hydroxypropyl methylcellulose or polyethylene oxide, may also be suitable as materials for drug containing particles or particles. Other polymers include, but are not limited to, polyanhydrides, poly (ester anhydrides), polyhydroxy acids, such as polylactide (PLA), polyglycolide (PGA), poly(lactide-co-glycolide) (PLGA), poly-3-hydroxybut rate (PHB) and copolymers thereof, poly-4-hydroxybutyrate (P4HB) and copolymers thereof, polycaprolactone and copolymers thereof, and combinations thereof. In some embodiments, both agents are incorporated into the same particles and are formulated for release at different times and/or over different time periods. For example, in some embodiments, one of the agents is released entirely from the particles before release of the second agent begins. In other embodiments, release of the first agent begins followed by release of the second agent before the all of the first agent is released. In still other embodiments, both agents are released at the same time over the same period of time or over different periods of time.

### 1. Formulations for Parenteral Administration

Compounds and pharmaceutical compositions thereof can be administered in an aqueous solution, by parenteral injection. The formulation may also be in the form of a suspension or emulsion. In general, pharmaceutical compositions are provided including effective amounts of the active agent(s) and optionally include pharmaceutically acceptable diluents, preservatives, solubilizers, emulsifiers, adjuvants and/or carriers. Such compositions include diluents sterile water, buffered saline of various buffer content (e.g., Tris-HCl, acetate, phosphate), pH and ionic strength; and optionally, additives such as detergents and solubilizing agents (e.g., TWEEN® 20, TWEEN® 80 also referred to as POLYSORBATE® 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### 2. Oral Immediate Release Formulations

Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydorxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, POLOXAMER® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

### 3. Extended Release Dosage Forms

The extended release formulations are generally prepared as diffusion or osmotic systems, for example, as described in “Remington—The science and practice of pharmacy” (20th ed., Lippincott Williams & Wilkins, Baltimore, MD, 2000). A diffusion system typically consists of two types of devices, reservoir and matrix, and is well known and described in the art. The matrix devices are generally prepared by compressing the drug with a slowly dissolving polymer carrier into a tablet form. The three major types of materials used in the preparation of matrix devices are insoluble plastics, hydrophilic polymers, and fatty compounds. Plastic matrices include, but not limited to, methyl acrylate-methyl methacrylate, polyvinyl chloride, and polyethylene. Hydrophilic polymers include, but are not limited to, methylcellulose, hydroxypropylcellulose, hydroxypropylmethylcellulose, sodium carboxymethylcellulose, and carbopol 934, polyethylene oxides. Fatty compounds include, but are not limited to, various waxes such as carnauba wax and glyceryl tristearate.

Alternatively, extended release formulations can be prepared using osmotic systems or by applying a semi-permeable coating to the dosage form. In the latter case, the desired drug release profile can be achieved by combining low permeable and high permeable coating materials in suitable proportion.

The devices with different drug release mechanisms described above could be combined in a final dosage form having single or multiple units. Examples of multiple units include multilayer tablets, capsules containing tablets, beads, granules, etc.

An immediate release portion can be added to the extended release system by means of either applying an immediate release layer on top of the extended release core using coating or compression process or in a multiple unit system such as a capsule containing extended and immediate release beads.

Extended release tablets containing hydrophilic polymers are prepared by techniques commonly known in the art such as direct compression, wet granulation, or dry granulation processes. Their formulations usually incorporate polymers, diluents, binders, and lubricants as well as the active pharmaceutical ingredient. The usual diluents include inert powdered substances such as any of many different kinds of starch, powdered cellulose, especially crystalline and microcrystalline cellulose, sugars such as fructose, mannitol and sucrose, grain flours and similar edible powders. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders include substances such as starch, gelatin and sugars such as lactose, fructose, and glucose. Natural and synthetic gums, including acacia, alginates, methylcellulose, and polyvinylpyrrolidine can also be used. Polyethylene glycol, hydrophilic polymers, ethylcellulose and waxes can also serve as binders. A lubricant is necessary in a tablet formulation to prevent the tablet and punches from sticking in the die. The lubricant is chosen from such slippery solids as talc, magnesium and calcium stearate, stearic acid and hydrogenated vegetable oils.

Extended release tablets containing wax materials are generally prepared using methods known in the art such as a direct blend method, a congealing method, and an aqueous dispersion method. In a congealing method, the drug is mixed with a wax material and either spray-congealed or congealed and screened and processed.

### 4. Delayed Release Dosage Forms

Delayed release formulations are created by coating a solid dosage form with a film of a polymer which is insoluble in the acid environment of the stomach, and soluble in the neutral environment of small intestines.

The delayed release dosage units can be prepared, for example, by coating a drug or a drug-containing composition with a selected coating material. The drug-containing composition may be, e.g., a tablet for incorporation into a capsule, a tablet for use as an inner core in a “coated core” dosage form, or a plurality of drug-containing beads, particles or granules, for incorporation into either a tablet or capsule. Preferred coating materials include bioerodible, gradually hydrolyzable, gradually water-soluble, and/or enzymatically degradable polymers, and may be conventional “enteric” polymers. Enteric polymers, as will be appreciated by those skilled in the art, become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename EUDRAGIT®. (Rohm Pharma; Westerstadt, Germany), including EUDRAGIT®. L30D-55 and L100-55 (soluble at pH 5.5 and above), EUDRAGIT®. L-100 (soluble at pH 6.0 and above), EUDRAGIT®. S (soluble at pH 7.0 and above, as a result of a higher degree of esterification), and EUDRAGITS*. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

The preferred coating weights for particular coating materials may be readily determined by those skilled in the art by evaluating individual release profiles for tablets, beads and granules prepared with different quantities of various coating materials. It is the combination of materials, method and form of application that produce the desired release characteristics, which one can determine only from the clinical studies.

The coating composition may include conventional additives, such as plasticizers, pigments, colorants, stabilizing agents, glidants, etc. A plasticizer is normally present to reduce the fragility of the coating, and will generally represent about 10 wt. % to 50 wt. % relative to the dry weight of the polymer. Examples of typical plasticizers include polyethylene glycol, propylene glycol, triacetin, dimethyl phthalate, diethyl phthalate, dibutyl phthalate, dibutyl sebacate, triethyl citrate, tributyl citrate, triethyl acetyl citrate, castor oil and acetylated monoglycerides. A stabilizing agent is preferably used to stabilize particles in the dispersion. Typical stabilizing agents are nonionic emulsifiers such as sorbitan esters, polysorbates and polyvinylpyrrolidone. Glidants are recommended to reduce sticking effects during film formation and drying, and will generally represent approximately 25 wt. % to 100 wt. % of the polymer weight in the coating solution. One effective glidant is talc. Other glidants such as magnesium stearate and glycerol monostearates may also be used. Pigments such as titanium dioxide may also be used. Small quantities of an anti-foaming agent, such as a silicone (e.g., simethicone), may also be added to the coating composition.

### 5. Formulations for Mucosal and Pulmonary

### Administration

Active agent(s) and compositions thereof can be formulated for pulmonary or mucosal administration. The administration can include delivery of the composition to the lungs, nasal, oral (sublingual, buccal), vaginal, or rectal mucosa. In a particular embodiment, the composition is formulated for and delivered to the subject sublingually.

In one embodiment, the compounds are formulated for pulmonary delivery, such as intranasal administration or oral inhalation. The respiratory tract is the structure involved in the exchange of gases between the atmosphere and the blood stream. The lungs are branching structures ultimately ending with the alveoli where the exchange of gases occurs. The alveolar surface area is the largest in the respiratory system and is where drug absorption occurs. The alveoli are covered by a thin epithelium without cilia or a mucus blanket and secrete surfactant phospholipids. The respiratory tract encompasses the upper airways, including the oropharynx and larynx, followed by the lower airways, which include the trachea followed by bifurcations into the bronchi and bronchioli. The upper and lower airways are called the conducting airways. The terminal bronchioli then divide into respiratory bronchiole, which then lead to the ultimate respiratory zone, the alveoli, or deep lung. The deep lung, or alveoli, is the primary target of inhaled therapeutic aerosols for systemic drug delivery.

Pulmonary administration of therapeutic compositions composed of low molecular weight drugs has been observed, for example, beta-androgenic antagonists to treat asthma. Other therapeutic agents that are active in the lungs have been administered systemically and targeted via pulmonary absorption. Nasal delivery is considered to be a promising technique for administration of therapeutics for the following reasons: the nose has a large surface area available for drug absorption due to the coverage of the epithelial surface by numerous microvilli, the subepithelial layer is highly vascularized, the venous blood from the nose passes directly into the systemic circulation and therefore avoids the loss of drug by first-pass metabolism in the liver, it offers lower doses, more rapid attainment of therapeutic blood levels, quicker onset of pharmacological activity, fewer side effects, high total blood flow per cm³, porous endothelial basement membrane, and it is easily accessible.

The term aerosol as used herein refers to any preparation of a fine mist of particles, which can be in solution or a suspension, whether or not it is produced using a propellant. Aerosols can be produced using standard techniques, such as ultrasonication or high-pressure treatment.

Carriers for pulmonary formulations can be divided into those for dry powder formulations and for administration as solutions. Aerosols for the delivery of therapeutic agents to the respiratory tract are known in the art. For administration via the upper respiratory tract, the formulation can be formulated into a solution, e.g., water or isotonic saline, buffered or un-buffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2. One skilled in the art can readily determine a suitable saline content and pH for an innocuous aqueous solution for nasal and/or upper respiratory administration.

Preferably, the aqueous solution is water, physiologically acceptable aqueous solutions containing salts and/or buffers, such as phosphate buffered saline (PBS), or any other aqueous solution acceptable for administration to an animal or human. Such solutions are well known to a person skilled in the art and include, but are not limited to, distilled water, de-ionized water, pure or ultrapure water, saline, phosphate-buffered saline (PBS). Other suitable aqueous vehicles include, but are not limited to, Ringer's solution and isotonic sodium chloride. Aqueous suspensions may include suspending agents such as cellulose derivatives, sodium alginate, polyvinyl-pyrrolidone and gum tragacanth, and a wetting agent such as lecithin. Suitable preservatives for aqueous suspensions include ethyl and n-propyl p-hydroxybenzoate.

In another embodiment, solvents that are low toxicity organic (i.e. nonaqueous) class 3 residual solvents, such as ethanol, acetone, ethyl acetate, tetrahydrofuran, ethyl ether, and propanol may be used for the formulations. The solvent is selected based on its ability to readily aerosolize the formulation. The solvent should not detrimentally react with the compounds. An appropriate solvent should be used that dissolves the compounds or forms a suspension of the compounds. The solvent should be sufficiently volatile to enable formation of an aerosol of the solution or suspension. Additional solvents or aerosolizing agents, such as freons, can be added as desired to increase the volatility of the solution or suspension.

In one embodiment, compositions may contain minor amounts of polymers, surfactants, or other excipients well known to those of the art. In this context, “minor amounts” means no excipients are present that might affect or mediate uptake of the compounds in the lungs and that the excipients that are present are present in amount that do not adversely affect uptake of compounds in the lungs.

Dry lipid powders can be directly dispersed in ethanol because of their hydrophobic character. For lipids stored in organic solvents such as chloroform, the desired quantity of solution is placed in a vial, and the chloroform is evaporated under a stream of nitrogen to form a dry thin film on the surface of a glass vial. The film swells easily when reconstituted with ethanol. To fully disperse the lipid molecules in the organic solvent, the suspension is sonicated. Nonaqueous suspensions of lipids can also be prepared in absolute ethanol using a reusable PARI LC Jet+ nebulizer (PARI Respiratory Equipment, Monterey, CA).

Dry powder formulations (“DPFs”) with large particle size have improved flowability characteristics, such as less aggregation, easier aerosolization, and potentially less phagocytosis. Dry powder aerosols for inhalation therapy are generally produced with mean diameters primarily in the range of less than 5 microns, although a preferred range is between one and ten microns in aerodynamic diameter. Large “carrier” particles (containing no drug) have been co-delivered with therapeutic aerosols to aid in achieving efficient aerosolization among other possible benefits.

Polymeric particles may be prepared using single and double emulsion solvent evaporation, spray drying, solvent extraction, solvent evaporation, phase separation, simple and complex coacervation, interfacial polymerization, and other methods well known to those of ordinary skill in the art. Particles may be made using methods for making microspheres or microcapsules known in the art. The preferred methods of manufacture are by spray drying and freeze drying, which entails using a solution containing the surfactant, spraying to form droplets of the desired size, and removing the solvent.

The particles may be fabricated with the appropriate material, surface roughness, diameter and tap density for localized delivery to selected regions of the respiratory tract such as the deep lung or upper airways. For example, higher density or larger particles may be used for upper airway delivery. Similarly, a mixture of different sized particles, provided with the same or different active agents may be administered to target different regions of the lung in one administration.

### 6. Topical and Transdermal Formulations

Transdermal formulations may also be prepared. These will typically be gels, ointments, lotions, sprays, or patches, all of which can be prepared using standard technology. Transdermal formulations can include penetration enhancers.

### III. Immunogenic Compositions and Vaccines, and Methods of Use

### A. Compositions

Typically, an immunogenic composition includes an adjuvant, an antigen, a LAIP, or a combination thereof. The combination of an adjuvant and an antigen can be referred to as a vaccine. When administered to a subject in combination, the adjuvant and antigen can be administered in separate pharmaceutical compositions, or they can be administered together in the same pharmaceutical composition. As described herein, the disclosed proteins (and fragments and variants thereof) can serve as the antigen component of an immunogenic composition or vaccine formulation. Additionally, the composition can include an adjuvant. Thus, in some embodiments, the composition includes both an antigen/LAIP and an adjuvant. Two or more different antigens, one or more different adjuvants, or combinations thereof, can be used or combined.

### 1. Antigens

Antigens are compounds that are specifically bound by antibodies or T lymphocyte antigen receptors. They stimulate production of or are recognized by antibodies. Sometimes antigens are part of the host itself in an autoimmune disease. An immunogen is an antigen (or adduct) that is able to trigger a humoral or cell-mediated immune response. It first initiates an innate immune response, which then causes the activation of the adaptive immune response. An antigen binds the highly variable immunoreceptor products (B cell receptor or T cell receptor) once these have been generated. Immunogens are those antigens, termed immunogenic, capable of inducing an immune response. Thus, an immunogen is necessarily an antigen, but an antigen may not necessarily be an immunogen. For brevity, the disclosed antigenic and vaccines composition are typically referred to as having or encoding an antigen. However, unless specifically indicated otherwise, any of the antigens can also be an immunogenic (i.e., an immunogen). Thus, all the disclosure of compositions and methods of use related to antigenic compositions and vaccines is also expressly provided with respect to immunogen unless indicated to the contrary.

The EIAG encoded hypothetical proteins, and antigenic fragments thereof, and nucleic acids (e.g., mRNA) encoding them, are provided for use in inducing immune response thereto. They typically co-express strongly with proteins that are bloodstage vaccine candidates and currently at different stages of trials (44). For example, between the year 2000-2015, more than 30 blood stage vaccines registered in ClinicalTrials.gov have completed and with majority of the vaccine targeting the MSP1 and AMA1. The other candidates are EBA-175 and MSP3. However, because of their redundancy in their function or due to polymorphism in the genes encoding these proteins in the natural population, the trials were not successful. Recently, two other vaccine candidates have been proposed that are important for the parasite invasion are PfRH5 and double-candidates AMA1-RON2 complex. Interestingly, all these genes are strongly co-expressed with the 14 EIAGs supporting a conclusions that these proteins are viable vaccine candidates.

Thus, immunogenic compositions and vaccine including an effective amount an EIAG protein (e.g., any of the proteins of Tables 1 and/or 2) or variant thereof with at least 65 70, 75, 80, 85, 90, or 95 percent sequence identity thereto, or a fragment thereof, or a nucleic encoding the same, to induce an immune response thereto are provided. The immunogenic compositions and vaccines can be used in methods of treating and preventing parasitic infections, optionally, but preferably to the species of parasite from which the EIAG originates.

### 2. Adjuvants

Immunologic adjuvants stimulate the immune system's response to a target antigen, but do not provide immunity themselves. Adjuvants can act in various ways in presenting an antigen to the immune system. Adjuvants can act as a depot for the antigen, presenting the antigen over a longer period of time, thus maximizing the immune response before the body clears the antigen. Examples of depot type adjuvants are oil emulsions. An adjuvant can also act as an irritant, which engages and amplifies the body's immune response.

The adjuvant may be without limitation alum (e.g., aluminum hydroxide, aluminum phosphate); saponins purified from the bark of the Q. saponaria tree such as Quil A (a mixture of more than 25 different saponin molecules), or subcombinations or individual molecules thereof such as QS21 (a glycolipid that elutes in the 21st peak with HPLC fractionation; Antigenics, Inc., Worcester, Mass.); poly[di(carboxylatophenoxy) phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic).

Adjuvants may be TLR ligands, such as those discussed above. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

The adjuvant can also be oil emulsions (e.g., Freund's adjuvant); saponin formulations; virosomes and viral-like particles; bacterial and microbial derivatives; immunostimulatory oligonucleotides; ADP-ribosylating toxins and detoxified derivatives; alum; BCG; mineral-containing compositions (e.g., mineral salts, such as aluminium salts and calcium salts, hydroxides, phosphates, sulfates, etc.); bioadhesives and/or mucoadhesives; microparticles; liposomes; polyoxyethylene ether and polyoxyethylene ester formulations; polyphosphazene; muramyl peptides; imidazoquinolone compounds; and surface active substances (e.g. lysolecithin, pluronic polyols, polyanions, peptides, oil emulsions, keyhole limpet hemocyanin, and dinitrophenol).

Adjuvants may also include immunomodulators such as cytokines, interleukins (e.g., IL-1, IL-2, IL-4, IL-5, IL-6, IL-7, IL-12, etc.), interferons (e.g., interferon-.gamma.), macrophage colony stimulating factor, and tumor necrosis factor.

Immunostimulatory complexes called ISCOMs are particulate antigen delivery systems having antigen, cholesterol, phospholipid and saponin (Quil A or other saponin) with potent immunostimulatory activity. ISCOMATRIX® is a particulate adjuvant having cholesterol, phospholipids and saponins (Quil A) but without containing antigen. See, e.g., U.S. Pat. No. 9,149,520, Sun, et al., Volume 27, Issue 33, 16 Jul. 2009, Pages 4388-4401, and Morelli, et al., J Med Microbiol. 2012 July; 61(Pt 7):935-43. doi: 10.1099/jmm.0.040857-0. Epub 2012 Mar. 22. This adjuvant has principally the same structure as ISCOMs, consisting of perforated cage-like particles of approximately 40 nm in diameter. The antigens can be formulated with ISCOMATRIX® to produce vaccines capable of antigen presentation and immunostimulants similar to ISCOMs-type formulations, but with a wider range of applicability, since its use is not limited to hydrophobic membrane proteins. Modifications of ISCOMs formulations and ISCOMATRIX® have also been developed to achieve a better association of some antigens, such as described in WO 98/36772.

ISCOMs and ISCOMATRIX® combine the advantages of a particulate delivery system with the in situ presence of an adjuvant (Quil A) and consequently have been found to be more immunogenic than other colloidal systems such as liposomes and protein micelles. Formulations of ISCOMs and ISCOMATRIX® retained the adjuvant activity of the Quil A, while increasing its stability, reducing its hemolytic activity, and producing less toxicity. They also generate a similar immune response to the one obtained by immunizing with simple mixtures of antigen and saponin, but allow for the use of substantially smaller amounts of antigen. Several ISCOMs-type vaccine formulations or containing ISCOMATRIX® have been approved for veterinary use, for example against equine influenza virus.

Other liposomal systems mainly composed of saponins from Q. saponaria and sterols (primarily cholesterol) have been described, one of which is referred to as ASO1B. See, e.g., WO 96/33739, being also formulated as emulsions such as described in US 2005/0220814. See, also, U.S. Published Application No. 2011/0206758.

Iscomatrix-like adjuvants such as ISCOMATRIX® are thought to function via canonical inflammasome activation and subsequent release of pro-inflammatory cytokines such as IL-18 and IL-1β (Wilson, et al., Journal of immunology. 2014; 192(7):3259-68. doi: 10.4049/jimmunol.1302011. PubMed PMID: 24610009). This mechanism is thought to be mediated at least in-part by endosomal degradation and the release of NRLP3-activating cathepsin proteases into the cytosol.

B. Methods of Inducing an Immune Response, Immunity, and/or Antibody Production

Methods of inducing an immune response in a subject (e.g., a human) by administering to the subject a therapeutically effective amount of the disclosed immunogenic or vaccine compositions are provided. The immune response can be induced, increased, or enhanced by the composition compared to a control (e.g., absence of the composition or presence of another composition).

In some embodiments, the disclosed antigens or LAIP increase a B cell response, for example increasing antigen binding to B cell receptors, increased antigen internalization by B cells, increased calcium release, increased pSyk phosphorylation, or a combination thereof. In some embodiments, B cell proliferation and/or differentiation is increased.

The methods include administering the LA-ΔPFAP2-MRP malaria parasite to the subject. In one embodiment, the administration is not followed by administration of an antimalarial agent that is capable of preventing/blocking parasite growth. The subject is preferably a mammal, such as a human subject.

The compositions in some forms include LA-ΔPFAP2-MRP Plasmodium, such as P. falciparum, P. vivax, P. ovale, P. knowlesi, and P. malariae.

In some forms, the subject is administered a composition containing an effective amount of sporozoite stage LA-ΔPFAP2-MRP Plasmodium. In some forms the subject is administered a composition containing an effective amount of merozoites stage LA-ΔPFAP2-MRP Plasmodium. In some forms the LA-ΔPFAP2-MRP Plasmodium is a chemically attenuated LA-ΔPFAP2-MRP Plasmodium. In some forms the LA-ΔPFAP2-MRP Plasmodium is a genetically attenuated LA-ΔPFAP2-MRP Plasmodium. In some forms the LA-ΔPFAP2-MRP Plasmodium has been irradiated to provide attenuation.

In certain embodiments, a pharmaceutical composition comprising one or more species of live Plasmodium sporozoite-stage parasites is co-administered with an adjuvant such as a glycolipid adjuvant. Useful glycolipid analogs are disclosed for example in U.S. Pat. No. 9,642,909. In some embodiments, the co-administration is by the same or a different route of administration. For example, a pharmaceutical composition comprising one or more species of LA-ΔPFAP2-MRP Plasmodium sporozoite-stage parasites administered by an intravenous, intramuscular, intradermal, or subcutaneous route can be co-administered with a glycolipid adjuvant administered by an intravenous, intramuscular, intradermal, or subcutaneous route.

In some forms the subject is administered one or more doses or a pharmaceutical composition containing no more than 150,000 sporozoites: no more than 50,000 sporozoites or no more than 25,000 sporozoites. In some forms no more than 3 doses are administered, no more than two doses are administered or no more than one dose is administered.

In some embodiments, a disclosed composition is administered to a subject in need thereof in an effective amount to induce an antigen-specific antibody response (e.g., IgG, IgG2a, IgG1, or a combination thereof), increase a response in germinal centers (e.g., increase the frequency of germinal center B cells, increase frequencies and/or activation T follicular helper (Tfh) cells, increase B cell presence or residence in dark zone of germinal center or a combination thereof), increase plasmablast frequency, increase inflammatory cytokine expression (e.g., IL-6, IFN-γ, IFN-α, IL-1β, TNF-α, CXCL10 (IP-10), or a combination thereof), or a combination thereof.

In some embodiments, the administration of the composition alternatively or additionally induces a B-memory cell response in subjects administered the composition compared to a control. A B-memory cell response is intended to mean an increased frequency of peripheral blood B lymphocytes capable of differentiation into antibody-secreting plasma cells upon antigen encounter.

In some embodiments, the compositions can induce an effector cell response such as a CD4 or CD8 T-cell immune response, against at least one of the component antigen(s) or antigenic compositions compared to the effector cell response obtained under control conditions (e.g., absence of the composition or presence of another composition). The term “improved effector cell response” refers to a higher effector cell response such as a CD8 or CD4 response obtained in a human patient after administration of a disclosed composition than that obtained under control conditions.

The described compositions may be administered as part of prophylactic vaccines or immunogenic compositions which confer resistance in a subject to subsequent exposure to infectious agents, or as part of therapeutic vaccines, which can be used to initiate or enhance a subject's immune response to a pre-existing antigen.

The desired outcome of a prophylactic or therapeutic immune response may vary according to the disease or condition to be treated, or according to principles well known in the art. For example, an immune response against an infectious agent may completely prevent colonization and replication of an infectious agent, affecting “sterile immunity” and the absence of any disease symptoms. However, a vaccine against infectious agents may be considered effective if it reduces the number, severity or duration of symptoms; if it reduces the number of individuals in a population with symptoms; or reduces the transmission of an infectious agent. Similarly, immune responses may completely treat a disease, may alleviate symptoms, or may be one facet in an overall therapeutic intervention against a disease.

The disclosed compositions may be used in methods of inducing protective immunity against an infectious agent, disease, or condition by administering to a subject (e.g., a human) a therapeutically effective amount of the compositions. “Protective immunity” or “protective immune response” refers to immunity or eliciting an immune response against an infectious agent, which is exhibited by a subject (e.g., a human), that prevents or ameliorates an infection or reduces at least one symptom thereof.

Another disclosed method provides for inducing the production of neutralizing antibodies or inhibitory antibodies in a subject (e.g., a human) by administering any of the disclosed compositions to the subject. In some embodiments, a disclosed composition is administered to a subject in need thereof in an effective amount to increase an antigen-specific antibody response (e.g., IgA, IgD, IgE, IgM, IgG, IgG2a, IgG1, or a combination thereof).

The antibody response is important for preventing many infections and may also contribute to resolution of infection. For example, when a vertebrate (e.g., a human) is infected with a virus, antibodies are produced against many epitopes on multiple virus proteins. A subset of these antibodies can block virus infection by a process called neutralization. Antibodies can neutralize viral infectivity in a number of ways. They may interfere with virion binding to receptors (blocking viral attachment), block uptake into cells (e.g., blocking endocytosis), prevent uncoating of the genomes in endosomes, or cause aggregation of virus particles. Many enveloped viruses are lysed when antiviral antibodies and serum complement disrupt membranes.

### IV. Inhibitors and Methods of Use

### A. Inhibitors

The EIAG encoded hypothetical proteins, and the genes that encode them, are also provided as drug targets drug targets, preferably for inhibition thereof. As discussed in more detail elsewhere herein, the parasite invasion and egress processes involve signaling molecules such as kinases, proteases and many of them are drug candidates. For example, Plasmepsin IX45, an aspartic protease and SUB146, a subtilisin like serine protease have been shown to essential for parasite egress and are druggable candidates. Both SUB1 and Plamsmesin IX co-express strongly with all the 14 EIAGs. Therefore, it is believed that many of these EIAGs (i.e., from Table 1 and/or Table 2 or as described elsewhere herein, either singly or in combination) could be a new classes of proteins that could be a target for intervention strategies to control malaria.

Any suitable compound can be used. Most typically it is effective to reduce expression or activity of one or EIAG proteins in a subject in need thereof, alone or in combination with one or more additional active agents. In some embodiments, the compound is an inhibitory polypeptide; a small molecule or peptidomimedic, or an inhibitory nucleic acid that targets genomic or expressed EIAG nucleic acids (e.g., mRNA), or a vector that encodes an inhibitory nucleic acid. The compound can reduce the expression or bioavailability of an EIAG protein or nucleic acid. EIAG inhibition can be competitive, non-competitive, uncompetitive, or product inhibition. Thus, an EIAG inhibitor can directly inhibit EIAG, a EIAG inhibitor can inhibit another factor in a pathway that leads to induction, persistence, or amplification of EIAG expression, or a combination thereof.

The EIAG inhibitor can be a functional nucleic acid. Functional nucleic acids are nucleic acid molecules that have a specific function, such as binding a target molecule or catalyzing a specific reaction.

The functional nucleic acid molecules can act as effectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA or the genomic DNA of a target polypeptide or they can interact with the polypeptide itself. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Therefore the compositions can include one or more functional nucleic acids designed to reduce expression of an EIAG gene, or a gene product thereof. For example, the functional nucleic acid or polypeptide can be designed to target and reduce or inhibit expression or translation of An EIAG mRNA; or to reduce or inhibit expression, reduce activity, or increase degradation of An EIAG protein. In some embodiments, the composition includes a vector suitable for in vivo expression of the functional nucleic acid.

In some embodiments, a functional nucleic acid or polypeptide is designed to target a segment of the nucleic acid sequence of an EIAG mRNA sequence, or the complement thereof, or a genomic sequence corresponding therewith, or variants thereof having a nucleic acid sequence at least 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical thereto.

In some embodiments, the function nucleic acid hybridizes to the nucleic acid encoding an EIAG protein, or a complement thereof, for example, under stringent conditions.

Functional nucleic acid molecules can be divided into the following non-limiting categories: antisense molecules, siRNA, miRNA, aptamers, ribozymes, triplex forming molecules, RNAi, external guide sequences, and gene editing compositions. In some embodiments, the gene editing composition induces a single or a double strand break in the target cell's genome. One example is the CRISPR/Cas system. CRISPR (Clustered Regularly Interspaced Short Palindromic Repeats) is an acronym for DNA loci that contain multiple, short, direct repetitions of base sequences. The prokaryotic CRISPR/Cas system has been adapted for use as gene editing (silencing, enhancing or changing specific genes) for use in eukaryotes (see, for example, Cong, Science, 15:339(6121):819-823 (2013) and Jinek, et al., Science, 337(6096):816-21 (2012)). By transfecting a cell with the required elements including a Cas gene and specifically designed CRISPRs, the organism's genome can be cut and modified at any desired location. Methods of preparing compositions for use in genome editing using the CRISPR/Cas systems are described in detail in WO 2013/176772 and WO 2014/018423, which are specifically incorporated by reference herein in their entireties.

In general, “CRISPR system” refers collectively to transcripts and other elements involved in the expression of or directing the activity of CRISPR-associated (“Cas”) genes and/or proteins, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. One or more tracr mate sequences operably linked to a guide sequence (e.g., direct repeat-spacer-direct repeat) can also be referred to as pre-crRNA (pre-CRISPR RNA) before processing or crRNA after processing by a nuclease.

In some embodiments, a tracrRNA and crRNA are linked and form a chimeric crRNA-tracrRNA hybrid where a mature crRNA is fused to a partial tracrRNA via a synthetic stem loop to mimic the natural crRNA:tracrRNA duplex as described in Cong, Science, 15:339(6121):819-823 (2013) and Jinek, et al., Science, 337(6096):816-21 (2012)). A single fused crRNA-tracrRNA construct can also be referred to as a guide RNA or gRNA (or single-guide RNA (sgRNA)). Within an sgRNA, the crRNA portion can be identified as the ‘target sequence’ and the tracrRNA is often referred to as the ‘scaffold’. sgRNAs targeting the EIAG genes are expressly provided.

There are many resources available for helping practitioners determine suitable target sites once a desired DNA target sequence is identified. For example, numerous public resources, including a bioinformatically generated list of about 190,000 potential sgRNAs, targeting more than 40% of human exons, are available to aid practitioners in selecting target sites and designing the associate sgRNA to affect a nick or double strand break at the site. See also, crispr.u-psud.fr/, a tool designed to help scientists find CRISPR targeting sites in a wide range of species and generate the appropriate crRNA and sgRNA sequences.

Other gene editing compositions that can be used to target the EIAG genes include, but are not limited to, zinc finger nucleases (ZFNs), transcription activator-like effector nuclease (TALEN). See, e.g., WO 2011/072246, U.S. Pat. Nos. 6,140,081; 6,453,242; 6,534,261; 6,610,512; 6,746,838; 6,866,997; 7,067,617; U.S. Published Application Nos. 2002/0165356; 2004/0197892; 2007/0154989; 2007/0213269; and International Patent Application Publication Nos. WO 98/53059 and WO 2003/016496.

In some embodiments, the genome editing composition optionally includes a donor polynucleotide. The modifications of the target DNA due to NHEJ and/or homology-directed repair can be used to induce gene correction, gene replacement, gene tagging, transgene insertion, nucleotide deletion, gene disruption, gene mutation, etc.

### B. Methods of Treating Parasitic Infections

The disclosed inhibitors can be administered to subject in need thereof in an effective amount to treat or prevent parasitic infections including those caused by Plasmodium falciparum, as well as others, including those provided herein, particularly those that can infect and optionally cause disease in humans. In particularly embodiments, the compositions are used to treat or prevent malaria.

The subject can be administered the inhibitor in a dosage and for a duration sufficient to improve one or more symptoms of the infection or disease.

In some embodiments, the inhibitor is administered in combination with another active agent, optionally wherein the other active agent is also for treatment of the infection of disease (e.g., malaria). In some embodiments, the other active agent targets another aspect of the parasite or its life cycle. In some embodiments, the inhibitor is administered in combination with, Atovaquone/Proguanil (Malarone), Chloroquine, Doxycycline, Mefloquine, Primaquine, Tafenoquine (Arakoda™), or a combination thereof.

The route of administration can be oral, parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration or using bioerodible inserts and can be formulated in dosage forms appropriate for each route of administration. Administration can be local or systemic. Formulations are discussed above. The precise dosage will vary according to a variety of factors including but not limited to the inhibitor that is selected and subject-dependent variables (e.g., age, immune system health, clinical symptoms etc.).

### EXAMPLES

The Examples below provide the results of conditional gene knockout approach (Collins et al., 2013; Knuepfer et al., 2017), showing that PF3D7_1107800 codes for PfAP2-MRP (master regulator of pathogenesis), which is an essential ApiAP2 transcription factor required for malaria parasite blood-stage growth. Using a series of phenotypic, RNA-seq and ChIP-seq analyses, PfAP2-MRP was established as a master regulator of malaria pathogenicity factors that directly regulates (activates or suppresses) the majority of the genes involved in egress, invasion, exportome and antigenic variation at different intraerythrocytic developmental stages, by binding to their promoters, and indirectly through downstream regulators. PfAP2-MRP also binds to the promoter of about˜50% of PfAP2s present in the P. falciparum and regulate the expression of majority of them, which is believed to make PfAP2-MRP an up-stream regulator of transcriptional cascade that P. falciparum shows during its IDC.

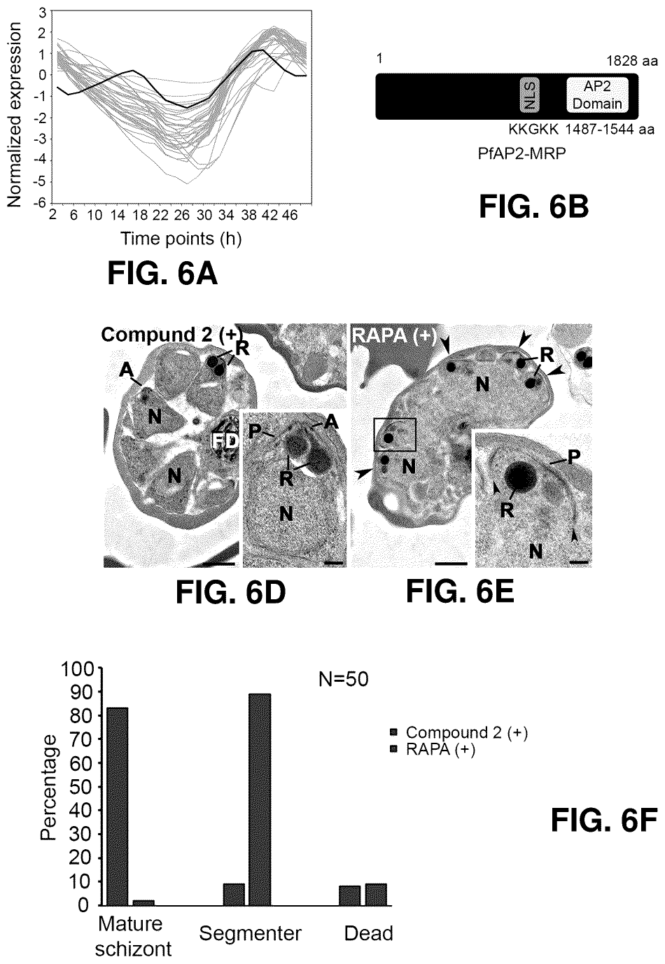

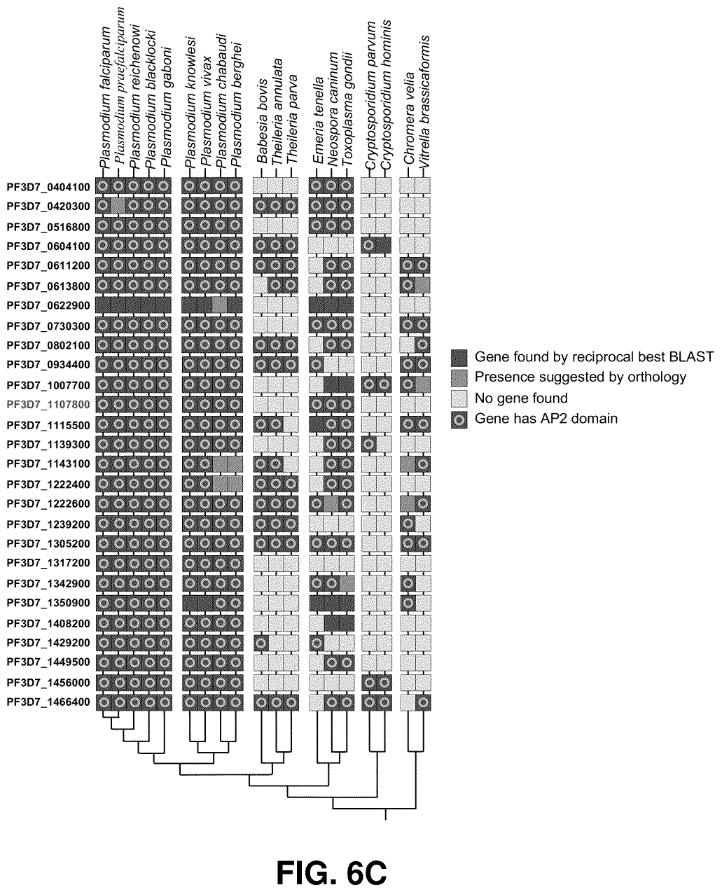

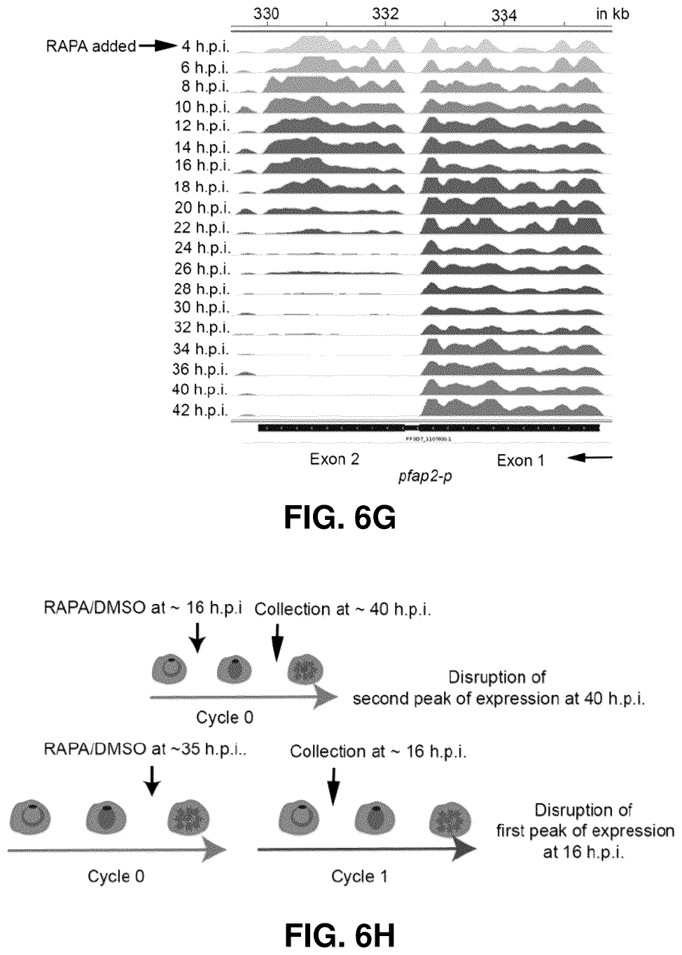

Subudhi et al., “Malaria parasites regulate intra-erythrocytic development duration via serpentine receptor 10 to coordinate with host rhythms, “Nat Commun., 11(1):2763 (2020). doi: 10.1038/s41467-020-16593-y, which is specifically incorporated by reference herein in its entirety, reports identification of 24 h rhythmic (circadian-like) transcripts in P. falciparum during the IDC. Out of 363 genes that showed a 24 h rhythmic expression i.e. with two peaks of expression in the 48 hour IDC, PfAP2-MRP was the only ApiAP2 DNA-binding protein. The first peak of expression at−16 h.p.i. coincides with maximal var gene family expression. The second peak at ˜40 h.p.i., is just before maximal expression of known invasion- and egress-associated genes, suggesting a role of PfAP2-MRP in regulating these genes (FIG. 6A). The second peak of PfAP2-MRP expression coincides with the time of maximal expression of the majority of known invasion- and egress-associated genes (FIG. 6A) that indicates the probable role of PfAP2-MRP as a regulator of these genes.

### Materials and Methods

### Parasite Culture Maintenance, Synchronization and Transfection

The DiCre-expressing P. falciparum clone 113 (Knuepfer et al., 2017) was maintained in human AB+erythrocytes at 37° C. in RPMI 1640 medium containing AlbumaxII (Invitrogen) supplemented with 2 mM L-glutamine. Parasites were either synchronized by sorbitol treatment or by purifying mature schizont stages using 70% (v/v) isotonic Percoll (GE Healthcare Life Sciences) before allowing reinvasion to occur, followed by sorbitol treatment. For transfection of plasmid constructs, purified Percoll-enriched mature schizonts (˜20 μl packed volume) were suspended in 100 μl of P3 primary cell solution (Lonza) containing 60 μg of linearized repair plasmid DNA (repair plasmid 1 and 2 separately in two separate transfection experiments) and 20 μg of pDC2 plasmid with the required cloned guide DNA and electroporated using an Amaxa 4D Nucleofactor (Lonza), using program FP158 as previously described (Moon et al., 2013). Drug selection was applied ˜20 h after transfection with 5 nm WR99210 (a kind gift of Jacobus Pharmaceuticals) for four days. Once sustained growth of drug-resistant transgenic parasites was observed, the cultures were treated with 1 μM 5-fluorocytosine (5-FC) provided as clinical Ancotil (MEDA) for four days. Transgenic parasite clones PfAP2M:loxP and PfAP2M-HA3:loxP were obtained by limiting dilution cloning in microplates. DiCre recombinase-mediated excision was induced by adding rapamycin to the culture of ring-stage parasites at a final concentration of 10 nM.

### Strategy to Perform Conditional Exon 2 Deletion (Gene Truncation) of PfAP2-MRP

To excise exon 2 of PfAP2-MRP that contains the DNA-binding AP2 domain and nuclear localization signal, endogenous exon 2 of PfAP2-MRP in the 113 DiCre-expressing P. falciparum clone was replaced with transgenic, “floxed” and HA-tagged and non-tagged forms of the exon 2 using two sequential Cas9-mediated genome editing procedures. In the first Cas9-mediated editing experiment, the single intron of PfAP2-MRP was replaced with SERA2-loxPint. The repair plasmid (repair plasmid 1) for this was synthesized commercially (GeneArt; Thermo) with recodonised sequences in the 2986-3024 bp and 3273-3305 bp regions of the PfAP2-MRP and 400 bp homology arms flanking the 5′ and 3′ regions of the intron. In the second Cas9-mediated editing experiment, cloned parasites with the integrated SERA2-loxPint were used to introduce a further loxP immediately after the stop codon of the PfAP2-MRP gene. The repair plasmid for this (repair plasmid 2) contained recodonized sequences in the 6261-6330 bp region of the gene, a XmaI restriction enzyme site followed by a loxP sequence just after the TAA stop codon, and 400 bp homology regions.

A triple HA-tagged version of the PfAP2-MRP was also prepared. For this, the stop codon was removed from the donor sequence (repair plasmid 2) by PCR amplification using primer pairs (Oligos P1 and P2) and digested with NotI and XmaI restriction enzymes. Repair plasmid 2 was also digested with NotI and XmaI restriction enzymes to remove the sequence with the stop codon from the plasmid backbone. Digested, amplified sequence without stop codon and digested plasmid backbone were ligated to get the repair plasmid 2 without stop codon after the PfAP2-MRP coding sequence. A triple HA tag sequence was PCR amplified from pFCSS plasmid with XmaI sites on both the sides of the HA tag sequence using oligos P5 and P6. XmaI digested repair plasmid 2 without stop codon was ligated with XmaI digested triple HA tag fragment to generate a triple HA-tagged encoding repair plasmid 2.

pDC2-Cas9-U6-hdhfr plasmid expressing spCas9 was used in this study. Guide RNA sequences were identified using Benchling. The TATTTATATTCTCAATTGAA (SEQ ID NO:61) and TTATATTCTCAATTGAATGG (SEQ ID NO:7) sequences targeting the 3′ end of the first exon, upstream of the TGG protospacer adjacent motif and 5′ end of the second exon upstream of the TGG protospacer respectively, were cloned into the pDC2-Cas9-U6-hdhfr (pDC2 plasmid 1) and used to target Cas9 in the first Cas9-mediated editing experiment (FIG. 1A). The CCCTTCAATAGATTCGCACA (SEQ ID NO:8) sequence towards the 3′ end of the second exon, upstream of the CGG protospacer adjacent motif, was 744 cloned into the pDC2-Cas9-U6-hdhfr (pDC2 plasmid 1) and used to target Cas9 in the second Cas9-mediated editing experiment (FIG. 1A).

Sequences of all the oligos and primers used are listed in Table 3.

TABLE 3
Sequences

| Oligo_Name | Sequence | Comment |
|---|---|---|
| Guide_1F | ATTGTATTTATATTCTCAATTGAA | Target to introduce |
| | (SEQ ID NO: 9) | sera2loxPint |
| Guide_1R | AAACTTCAATTGAGAATATAAATA | |
| | (SEQ ID NO: 10) | |
| Guide_3F | ATTGCCCTTCAATAGATTCGCACA | Target to introduce 3′ 3XHA |
| | (SEQ ID NO: 11) | and loxP |
| Guide_3R | AAACTGTGCGAATCTATTGAAGGG | |
| | (SEQ ID NO: 12) | |
| P1 | ATAGCGGCCGCATTTATTAGAT | Forward Primer to remove stop |
| | CATC ATTTGACAG | codon from repair plasmid 2 |
| | (SEQ ID NO: 13) | |
| P2 | AGTTATCCCGGGACTACTTATCT | Reverse Primer to remove stop |
| | TCTGGTC | codon from repair plasmid 2 |
| | (SEQ ID NO: 14) | |
| P3 | TGGAAATGTGGGTTCTACACCA | Forward primer in the up |
| | (SEQ ID NO: 15) | stream region of HR1 |
| P4 | TCTTAACGGAAATGGGCTCAC | Reverse primer down stream of |
| | (SEQ ID NO: 16) | HR 4 |
| P5 | TTTTGGACGCAGTTGCTGAC | Forward primer in the |
| | (SEQ ID NO: 17) | recodnized region R3 |
| P6 | CCTTTACCGCGGTCAAGCGTAAT | Reverse primer targeting HA |
| | (SEQ ID NO: 18) | tag sequences |

Primer used for qPCR

| | | |
|---|---|---|
| FIKK11_F | TTCATATGCACCTCCTGAATGT | PF3D7_1149300 |
| | (SEQ ID NO: 19) | |
| FIKK11_R | AGCCATAAGGGTTTACTATTCCA | PF3D7_1149300 |
| | (SEQ ID NO: 20) | |
| EBA175_F | ACGCTGTACGTGTGTCTAGG | PF3D7_0731500 |
| | (SEQ ID NO: 21) | |
| EBA175_R | TGCATAATTGGATTCTACGATCAGG | PF3D7_0731500 |
| | (SEQ ID NO: 22) | |
| EBA181_F | GGGGTTGTAAAGGAAAAAGAAGCA | PF3D7_0102500 |
| | (SEQ ID NO: 23) | |
| EBA181_R | TTTCTATCAGTCGTTTCAGATCCT | PF3D7_0102500 |
| | (SEQ ID NO: 24) | |
| RH4_F | TTTGTACCTCCGAATTCACCA | PF3D7_0424200 |
| | (SEQ ID NO: 25) | |
| RH4_R | AGTGTTTGTATGGAATGACAAGTT | PF3D7_0424200 |
| | (SEQ ID NO: 26) | |
| RH2B_F | AGGGATAATGGAACTGTCGTTT | PF3D7_1335300 |
| | (SEQ ID NO: 27) | |
| RH2B_R | TCTGTTTTCCCACCAACTAAACA | PF3D7_1335300 |
| | (SEQ ID NO: 28) | |
| SUB1_F | TCATGCAACTACCCCCTCGT | PF3D7_0507500 |
| | (SEQ ID NO: 29) | |
| SUB1_R | TCAAAGCTCCCTTCTCCTCAA | PF3D7_0507500 |
| | (SEQ ID NO: 30) | |
| MSP6_F | AAGGGAGCAAATGGTTTAACTGG | PF3D7_1035500 |
| | (SEQ ID NO: 31) | |
| MSP6_R | GAGCACCTCCTCCAAATTCCC | PF3D7_1035500 |
| | (SEQ ID NO: 32) | |
| Seryl_t_ | AAGTAGCAGGTCATCGTGGTT | Internal Control |
| lig_F | (SEQ ID NO: 33) | |
| Seryl_t_ | CAAAGTCATCAAGTTCGGCACA | Internal control |
| lig_R | (SEQ ID NO: 34) | |
| ETRAMP11_F | GCCTTGAATTTTATTGCCCCT | ETRAMP 11 |
| | (SEQ ID NO: 35) | |
| ETRAMP11_R | ACCTGATGCAATGGCAGAGTA | ETRAMP 11 |
| | (SEQ ID NO: 36) | |
| MDV1_F | AGAAAGCACTCTCAAGCCTCT | PF3D7_1216500 |
| | (SEQ ID NO: 37) | |
| MDV1_R | TCCGTTTCTTCATTAGCATTTCCG | PF3D7_1216500 |
| | (SEQ ID NO: 38) | |
| Pfpuf2_F | AGGAGGGGCAAGTGCATATT | PF3D7_0417100 |
| | (SEQ ID NO: 39) | |
| Pfpuf2_R | ACTTTGCGCTACATAACTACCA | PF3D7_0417100 |
| | (SEQ ID NO: 40) | |
| PfS16_F | CTTCGCTTTTGCAAACCTGG | PF3D7_0406200 |
| | (SEQ ID NO: 41) | |
| PfS16_R | TGAAGGGGATCCTTTTCCAGC | PF3D7_0406200 |
| | (SEQ ID NO: 42) | |
| 1462500_F | TGCATCCAAACAAATGAAGAGGA | PF3D7_1462500 |
| | (SEQ ID NO: 43) | |
| 1462500_R | ATTGGTATGCACTGGTATGTTTTT | PF3D7_1462500 |
| | (SEQ ID NO: 44) | |
| 714100_F | AGGTGCAGAATTTCAAGAAATGGA | PF3D7_0714100 |
| | (SEQ ID NO: 45) | |
| 714100_R | TTTTGAGAAAACATTCTGTGTTGC | PF3D7_0714100 |
| | (SEQ ID NO: 46) | |
| LSD2_F | CCATTCGAGCTCTGAGTCTGT | PF3D7_0801900 |
| | (SEQ ID NO: 47) | |
| LSD2_R | CCCCACCTCTTCCATATCCG | PF3D7_0801900 |
| | (SEQ ID NO: 48) | |

Primers used for Chip qPCR

| | | |
|---|---|---|
| RAP1_chip_F | ATGCACCGATAAGCATTACAC | PF3D7_1410400 |
| | (SEQ ID NO: 49) | |
| RAP1_chip_R | TATGATGTGAATATGTATAATT | PF3D7_1410400 |
| | CCCCGTC | |
| | (SEQ ID NO: 50) | |
| RhopH2_chip_F | CAATTCTGCAAGCATTTCAAT | PF3D7_0929400 |
| | (SEQ ID NO: 51) | |
| RhopH2_chip_R | CTTGGATGAGAGCACCGAA | PF3D7_0929400 |
| | (SEQ ID NO: 52) | |
| RhopH3_chip_F | TAATATGTACGCAAGGTGCAC | PF3D7_0905400 |
| | (SEQ ID NO: 53) | |
| RhopH3_chip_R | ATTTCTGCACTAAGGATGTATTA | PF3D7_0905400 |
| | (SEQ ID NO: 54) | |
| AP2I_RON3-F1 | CAAATCATTCTTTACCTTATGTT | PF3D7_1252100 |
| | (SEQ ID NO: 55) | |
| AP2I_RON3-R1 | TTTATCTTTTTGTGCACAGACC | PF3D7_1252100 |
| | (SEQ ID NO: 56) | |
| AP2I_RALP1-F1 | CTTATAAGAACGAAATAAGTGTG | PF3D7_0722200 |
| | (SEQ ID NO: 57) | |
| AP2I_RALP1-R1 | TGCTCAATATAAAATAAGCACTG | PF3D7_0722200 |
| | (SEQ ID NO: 58) | |
| AP2I_SIP2-F1 | CCGTTGATTCGTGCA | PF3D7_0604100 |
| | (SEQ ID NO: 59) | |
| AP2I_SIP2-R1 | ATACATATATGCATATGCACTC | PF3D7_0604100 |
| | (SEQ ID NO: 60) | |

### Flow Cytometry-Based Quantification of Parasitemia

Parasites were fixed in 0.1% glutaraldehyde and incubated for 30 minutes at 37° C., then stored at 4° C. until further use. Fixed parasites were stained with SyBR green (diluted 1:10,000) for 30 min at 37° C., then parasitemia was determined using an LSRFortessa flow cytometer (BD Biosciences, San Jose, CA, USA)).

### Invasion and Growth Assays

For invasion assay, the parasites were treated with DMSO or rapamycin at ˜16 h.p.i. in cycle 0 and mature schizonts in the same cycle were isolated using Percoll as described earlier and added to the culture at 5% parasitemia. Merozoites were allowed to invade for 4 hours under either static or vigorous shaking (250 rpm) conditions. Four hours after adding mature schizonts to the culture, the parasitemia was measured by flow cytometry. Three biological replicates per condition were used.

### Electron Microscopy

Compound 2 treated and RAPA treated highly synchronized parasites were allowed grow until they reach to the segmented schizont stage. Then the infected RBCs were fixed with 2.5% glutaraldehyde in cacodylate buffer (0.1 M, pH 7.4) for 48 h. A first Osmication was performed using reduced osmium (1:1 mixture of 2% osmium tetroxide and 3% potassium ferrocyanide) for 1 hour. After a quick wash with distilled water, a second osmication was performed with Osmium tetroxide 2% for 30 min. Samples were washed 3×min in water and placed in 1% Uranyl acetate for 12 hours at 4 degree C. Prior dehydration sample were washed 3×15 min in water. After pre embedding in agar 1%, samples were dehydrated in ethanol series (40% to 100%) and embedded in epoxy resin. Thin sections (100 nm to 150 nm thickness) were collected on copper grids and contrasted with lead citrate. Imaging was performed using a transmission electron microscope (TEM) operating at 300 kV (Titan Cryo Twin, Thermo Fisher Scientific). Images were 774 recorded on a 4k×4k CCD camera (Gatan Inc.).

### Nucleic Acid Extraction and Polymerase Chain Reaction

For DNA isolation, cells were pelleted and treated with 0.15% saponin in PBS for 10 min on ice, then washed twice with PBS. DNA was extracted from parasite pellets using DNeasy blood and tissue kit (Qiagen) following the manufacturer's instructions. For diagnostic PCR to check clones, GoTaq (Promega) DNA mastermix was used; for amplification of fragments (3×HA tag) used in construct design, Phusion high fidelity DNA polymerase (NEB) was used, and for amplification of fragments longer than >3 kb Platinum taq HiFi polymerase (Invitrogen) was used.

### PAGE, Immunoblotting and Immunofluorescence

Ring-stage parasites were treated with DMSO or rapamycin and subsequently mature schizonts (>45 h.p.i) were purified using 70% Percoll, then treated with 0.15% saponin in PBS and washed twice with PBS. Schizonts pellets were lysed by adding sample lysis buffer (1% NP40, 0.1% SDS, 150 mM NaCl) and 5 μg of each sample was separated under reducing conditions on Bis-Tris NuPAGE polyacrylamide gels and transferred to nitrocellulose membranes by electroblotting. Blots were blocked overnight in 5% milk powder (w/v) in phosphate buffered saline (PBS) containing 0.2% Tween-20. For the detection of 3×HA tagged PfAP2-MRP, the rat anti-HA mAb 3F10 (Sigma) was used at a 1:1000 dilution, followed by horseradish conjugated secondary antibody (1:2500). For the proteins tested, relevant primary antibodies were used (see below), then secondary HRP conjugated antibodies specific for mouse, rabbit or rat IgG (Biorad) were used at a dilution of 1:2500. The signal was developed using Immobilon Western Chemoluminescent HRP Substrate (Merck Millipore) and detected using Hyperfilm ECL film (GE Healthcare).

For immunofluorescence, thin films of parasite cultures were prepared and fixed in 4% paraformaldehyde in PBS for 20 min at room temperature. The cells were permeabilized with 0.1% Triton X100 in PBS for 5 min, then blocked with 3% bovine serum albumin (BSA) in PBS overnight at 4° C. before being probed with 804 relevant primary antibodies. Secondary Alexa Fluor 488- or 594-conjugated antibodies specific for mouse, rabbit or rat IgG (Invitrogen) were used at a 1:5000 dilution. Slides were examined using a Nikon Ni microscope with a 100×Plan Apo NA 1.45 objective; images were captured with an Orca Flash 4 digital camera, and prepared with Nikon NIS Elements and Adobe Photoshop software.

### Antibodies

The following antibodies and dilutions were used for Western blots in these studies. Rabbit anti-EBA175 (1:10,000), rat anti-MyoA (1:1,000), rat anti-BiP (1:1,000), rabbit anti-PTRAMP (1:4,000), rabbit anti-ARO (1:1,000), rabbit anti-AMA1 (1:10,000), and rabbit anti-SUB1 (1:1,000). Antibodies used in immunofluorescence were rabbit anti-GAP45 (1:1,000) and rabbit anti-MSP7 (1:1,000). Anti-EBA175 was obtained from MR4 (beiresources.org/MR4Home), anti-BiP was provided by Dr. Ellen Knuepfer, anti-SUB1 was a generous gift from Prof. Mike Blackman (Francis Crick Institute), and anti-AMA1 was a generous gift from Bart Faber and Clemens Kocken from the Primate Research centre in Rijswijk. All other antibodies were generated in the Holder laboratory and are now held and freely available at NIBSC-CFAR (please contact cfar@nibsc.org with any inquiries).

### Antibodies RNA Extraction and Strand-Specific RNA-Seq Library Preparation

Parasite cultures (˜0.5-2 ml depending on the asexual developmental stage) were pelleted at 2400 rpm for 3 min, lysed by adding 1 ml of Trizol (Sigma), then immediately stored at −80° C. until further use. Total RNA was isolated from Trizol lysed parasites according to manufacturer's instructions (Life Technologies). Strand-specific mRNA libraries were prepared from total RNA using TruSeq Stranded mRNA Sample Prep Kit LS (Illumina) according to the manufacturer's instructions. Briefly, for each sample 100-300 ng of total RNA was used to prepare the libraries. PolyA+mRNAs were captured from total RNA using oligo-T attached to magnetic beads. First strand synthesis was performed using random primers followed by second strand synthesis where dUTP was incorporated in place of dTTP to achieve strand-specificity. Double stranded cDNA ends were ligated with adaptors and the libraries were amplified by PCR for 15 cycles, before sequencing the libraries on Illumina HiSeq-4000 platform with paired-end 150 bp read chemistry according to manufacturer's instructions (Illumina).

### RNA-Seq Data Processing and Analysis

The quality of the raw reads was assessed using FASTQC (http://www.bioinformatics.babraham.ac.uk/projects/fastqc). Low-quality reads and Illumina adaptor sequences from the read ends were removed using TrimmomaticR⁴⁷. Processed reads were mapped to the P. falciparum 3D7 reference genome (release 40 in PlasmoDB-http://www.plasmoddb.org) using Hisat2⁴⁸ (V 2.1.0) with parameter “-rna-strandness FR”. Counts per feature were estimated using FeatureCounts⁴⁹. Raw read counts data were converted to counts per million (cpm), and genes were excluded if they failed to achieve a cpm value of 1 in at least one of the three replicates performed. Library sizes were scale-normalized by the TMM method using EdgeR software⁵⁰ and further subjected to linear model analysis using the voom function in the limma package⁵¹. Differential expression analysis was performed using DeSeq2⁵². Genes with a false discovery rate corrected P value (Benjamini-Hochberg procedure)<0.05 and log 2 fold change ≥1 or ≤−1 were considered as up-regulated or down regulated, respectively.

### Real-Time Quantitative PCR

Total RNA was treated with DNAase (TURBO DNase, Cat No. AM2238, Invitrogen) following the manufacturer's instructions. The removal of DNA was confirmed by performing PCR using housekeeping genes. cDNA (1 μg) was prepared from total RNA using LunaScript cDNA synthesis mix (NEB), diluted 5 times before using it for qRT-PCR. mRNA expression levels were estimated on a Quant Studio 3 real-time qPCR machine (Applied Biosystems) using Fast SYBR green master mix (Applied Biosystems, Cat. No. 4385612). Seryl-tRNA ligase (PF3D7_0717700) was used as the internal control to normalize mRNA levels. Specific amplification of the PCR product was verified by dissociation curve analysis and relative quantities of mRNA calculated using the ΔΔCt Method⁵³. PCR primers used in the real-time qRT-PCR experiment are listed in Table 1. For the var gene qRT-PCR experiment, primer sets targeting individual var genes described elsewhere⁵⁴ were used, including the primers targeting the seryl tRNA ligase gene (PF3D7_0717700) used as the internal control.

The ChIP assay was performed as described⁵⁵ with a few modifications. Parasite culture (50 ml) containing synchronized late stage schizonts (˜40 h.p.i.) at ˜5% parasitemia was centrifuged at 900 g for 4 min, and the cells were washed once with PBS. To the cell pellet, 25 ml of 0.15% saponin in PBS was added and incubated on ice for 10 min, followed by washing twice with cold PBS. Parasites were crosslinked for 10 min by adding methanol-free formaldehyde at 1% final concentration and incubated for 10 min at 37° C. with occasional shaking. The crosslinking reaction was quenched by adding 1.25 M glycine to achieve a final concentration of 0.125 M and incubated at 37° C. for another 5 min. Parasites were centrifuged for 10 min at 3250 g at 4° C., washed three times with DPBS, snap-frozen in liquid nitrogen, and stored at −80° C. until further use. Frozen formaldehyde-fixed parasites were thawed on ice for chromatin immunoprecipitation. One ml of nuclear extraction buffer (10 mM HEPES, 10 mM KCl, 0.1 mM EDTA, 0.1 mM EGTA, 1 mM DTT, 1×EDTA-free protease inhibitor cocktail [Roche]) was added to the tubes containing thawed parasites and incubated on ice for 30 min. After the incubation, 10% NP-40 was added to reach a final concentration of 0.25%, and the parasites were lysed by passing through a 26^{1/2} G needle seven times. Parasite nuclei were collected by centrifuging at 2500 g for 10 min at 4° C. Shearing of chromatin was carried out using the Covaris Ultra Sonicator (E220) for 14 min with the following settings; 5% duty cycle, 140 intensity peak incident power, 200 cycles per burst to obtain fragment size of 200 to 600 bp. Insoluble materials were removed by centrifuging the sheared chromatin for 10 min at 13500 g at 4° C. 30 μl of fragmented chromatin were stored as input at −80° C.

Fragmented chromatin was diluted 1:1 in ChIP dilution buffer (30 mM Tris-HCl pH 8.0, 0.1% SDS, 3 mM EDTA, 300 mM NaCl, 1% Triton X-100, EDTA-free protease inhibitor cocktail). Chromatin was incubated overnight with 6 μg of rabbit polyclonal anti-HA (Abcam no. ab9110) or, as control, the same amount of rabbit IgG isotype control (Cat. No. 10500c, Invitrogen). For histone marks H3K9me3, H3K9ac and H3K4me3, rabbit polyclonal anti H3K9me3 antibody (Millipore no. 07-442), rabbit polyclonal anti H3K9ac antibody (Millipore no. 07-352) and rabbit polyclonal anti H3K4me3 antibody (Abcam no. ab8580) were used respectively. The antibody-protein complex was recovered with protein A coupled to magnetic beads (Dynabeads, Invitrogen, Cat No. 10002D), followed by extensive washes with low salt immune complex wash buffer, high salt immune complex wash buffer (washes done at 4° C.), and TE buffer (washes done at RT). Chromatin was eluted with elution buffer (1% SDS, 0.1 M NaHCO₃) at 45° C. for 30 min with shaking. Immunoprecipitated chromatin and input were reverse crosslinked overnight at 45° C. by adding 5 M NaCl to a final concentration of 0.5 M. Samples were treated with RNaseA for 30 min at 37° C. followed by a 2 h incubation at 45° C. with proteinase K (final concentration 66 μg/ml). DNA was purified using ChIP DNA clean & concentrator (Zymo Research, Cat. No. D5205).

### PfAP2-MRP ChIP-Sequencing and Analysis

Libraries were prepared using NEBNext Ultra II DNA library kit following the manufacturer's instructions until the step of adapter ligation (Adapters were diluted at 1:20 ratio). Adapter ligated libraries were purified using AmpureXP beads. The libraries were amplified for a total of 6 PCR cycles (2 min at 98° C. initial denaturation; 6 cycles of 30 s at 98° C., 50 s at 62° C., final extension 5 min at 62° C.) using the KAPA HiFi HotStart Ready Mix (Kapa Biosystems). Amplified libraries were purified, and size selected for 350 bp inserts using AmpureXP beads and sequenced on the Illumina HiSeqX platform with 150 bp paired end read layouts. Low-quality reads and Illumina adaptor sequences from the read ends were removed using Trimmomatic⁴⁷. Quality trimmed reads were aligned to the P. falciparum genome (plasmodb.org, v3, release v32) using HiSat2. Duplicate reads were removed using samtools (markdup)⁵⁶. GC bias was corrected using deeptool's correctGCBias tool⁵⁷. For coverage plots of AP2-MRP 40 h.p.i. and 16 h.p.i. ChIP-seq experiments, deeptool's bamCompare tool was used to normalize the read coverage per base of the genome position (option ‘-bs 1’) in the respective input and ChIP samples or IgG and ChIP samples to the total number of reads in each library (—normalizeUsing RPKM). Normalized input coverage or IgG coverage per bin was subtracted from the ChIP values (option—operation subtract). Coverage plots were visualized using IGV genome browser⁵⁸.

ChIP-Peaks (q-value cutoff <0.05) were identified using macs2⁵⁹ by comparing the input with ChIP or IgG with ChIP with default settings but without prior peak modeling (option ‘-nomodel’), the fragment size set to 200 bp (option ‘-extsize 200’) and the genome size (option ‘-g’) set to 233332839. Robust common peaks between replicates were identified using bedtools ‘intersect’ (option −f 0.30−r)⁶⁰. Peak annotation was carried out using Homer's annotatePeaks.pl that assigned each peak with the nearest downstream gene. After intersecting, common peaks with peak score >50 were kept for further analysis. Enrichment heatmaps and profile plots were generated using the deepTools computeMatrix and plotHeatmap tools.

### Processing of Published PfAP2-I and PfAP2-G ChIP-Seq Data

PfAP2-I and PfAP2-G ChIP-seq published raw data^{30,31} were downloaded from ENA and processed exactly as the ChIP-seq data for PfAP2-MRP. ChIP-Peaks (q-value cutoff <0.05) were identified using macs2⁵⁹ by comparing the input with ChIP for both the replicates of PfAP2-G and PfAP2-I. Robust common peaks between PfAP2-MRP and PfAP2-I; PfAP2-MRP and PfAP2-MRP and between all three were identified using bedtools ‘intersect’ (option−f 0.30-r) to find peaks that overlapped at least 30%.

### Parasite Sample Preparation for Single-Cell RNA-Seq

For 40 h.p.i. time-point, tightly synchronous parasites were enriched using 63% Percol, washed twice with incomplete RPMI medium, and processed immediately on the 10× Chromium controller (10× Genomics, Pleasanton, CA). For 16 h.p.i. time point parasites were stained with Mitotracker Deep Red FM (Life Technologies, #M22426) for FACS analysis and flow sorting, respectively. Briefly, 50 μl of SYBR Green I stained RBCs were analyzed on BD LSR Fortessa Flow Cytometer with High Throughput sampler (BD Biosciences, San Jose, CA, USA) using BD FACS Diva Software v6.2 and 488 laser excitation/530 emission filter to determine the concentration of SYBR Green I positive cells per microliter. A BD Influx Cell Sorter (BD Biosciences, San Jose, CA, USA) with BD FACS Sortware v1.0.01 software was used to sort ˜40,000 MitoTracker Deep Red FM-positive RBC's using a 70 μm nozzle, a 640 nm laser excitation/670 nm emission filter and a pressure setting of 30 psi. Post-sorted cell concentration and quality were checked using a Countess® II Automated Cell Counter (Invitrogen) and FLoid™ Cell Imaging Station (ThermoFisher). Finally, labeled cells (i.e., SYBR Green I or MitoTracker Deep Red FM positive cells) were then loaded onto a 10× chip (Chip G) and processed immediately on the 10× Chromium controller (10× Genomics, Pleasanton, CA).

### Single-Cell RNA-Seq Library Preparation

Single-cell libraries were constructed using the 10× Genomics Chromium Next GEM Single Cell 3′ Reagent Kits v3.1 with Single Index Kit T Set A. Due to the extremely low RNA content of single-cell malaria parasite and the AT-rich genome (˜70% AT), modifications to the cDNA amplification and library preparation workflow were made accordingly. These modifications included—30×cDNA amplification cycles; taking 50% cDNA as input into library generation; reducing fragmentation time to 2 minutes; and changing the extension time to 65° C. during index PCR. Individual library Qc was performed using the BioAnalyzer HS DNA Assay kit (Agilent).

### Sequencing of Single-Cell RNA-Seq Library

Library concentration was determined with the KAPA Library Quantification Kit (ROCHE) using the QuantStudio3 Real-Time PCR systems (ThermoFisher) and assessed for fragment size using the BioAnalyzer HS DNA Assay kit (Agilent). Following library pooling in equimolar concentrations, a total of 1.2 nM library was sequenced on the Illumina NovaSeq 6000 with SP flow cell using version 1.5 chemistry as follows: Read 1-28 bp; Index read i7-8 bp; Index read i5-0 bp; and Read 2-91 bp.

### Single-Cell Transcriptome Alignment and Read Count Estimation

The Droplet-based sequencing reads were aligned to the Hybrid genome of Human hg38 and P. falciparum pd37 (PlasmoDB-46_Pfalciparum3D7_Genome.fasta) to remove any human transcript contamination. This was achieved using CellRanger standard pipeline using—nosecondary flag. The raw gene count matrix was subjected to various single-cell preprocessing steps separately.

### Preprocessing and Normalization

Primarily, the reads mapping to human genes were removed, followed by the identification and removal of empty droplets using the emptyDrops( ) function from R package dropletUtils v1.12.3⁶¹. This function determines whether the RNA content of a cell barcode differs considerably from the ambient background RNA present in each sample. Cells with FDR<=0.001 (Benjamini-Hochberg-corrected) were examined for subsequent investigation. The per-cell quality metrics were computed by the addPerCellQC function of the scuttle package v1.2.1⁶². The deconvolution approach in the computeSumFactors function of the Scran R package v1.20.1⁶³ was utilized to normalize cell-specific biases. The mitochondrial genes of Pfalciparum was keptsince the proportion of UMIs allocated to mitochondrial genes in both controls and knockouts were similar.

Further, the doublets identification was performed using the computeDoubletDensity function of the scDblFinder Bioconductor package v1.6.0 [Germain P (2021). scDblFinder: scDblFinder. R package version 1.6.0, https://github.com/plger/scDblFinder.]. This was achieved in three steps. a) The log normalization of counts was achieved using the logNormCounts function of scuttle package. b) The modelGeneVarByPoisson function of scran63 was then used to model the per-gene variance followed by c) doublet score calculation using the top 10% of highly variable genes (HVGs). The data was cleaned based on a 95% quantile cut-off. Additionally, the standard functions of the Seurat package⁶⁴ were also used to generate intuitive QC plots.

### Cell Type and Infection Stage Recognition:

The transcriptomic data at single-cell resolution about the malaria life cycle was obtained from the Malaria Cell Atlas. The phenotypic data for MCA was obtained from github.com/vhowick/MalariaCellAtlas/blob/master/Expression_Matrices/10×/pf10×IDC/pf10 xIDC_pheno.csv65. The SingleR package v1.6.1 was used to transfer labels from the reference atlas to each cell in our data. It identifies marker genes for each stage in the reference atlas and uses them to compute assignment scores (based on the Spearman correlation across markers) for each cell in the test dataset against each label in the reference. The top 20 marker genes were identified using the Wilcoxon ranked sum test66 The time-series data of the intra-erythrocytic development cycle (IDC) was obtained from14. To transfer the time-series labels from Bulk RNASeq, a subset of samples for both 16 h.p.i and 40 h.p.i was used to prevent label misassignments. Each biological replicate was considered a single cell and markers were identified using the “classic” method of SingleR. The correlation between cells of different stages was carried out using the CorrelateReference( ) function of the CHETAH R package v1.8.067. Similarly, an inter-sample correlation wasperformed between the transcriptional profiles of samples from different time points.

Integration of In-House Data with MCA

The cleaned data was then integrated with Malaria Cell Atlas Single Cell data using standard scRNAseq integration workflow as described elsewhere64. Briefly, an “integrated” data assay was created but first identifying the pairwise anchors using FindIntegrationAnchors followed by IntegrateData that exploit this anchor-set to combine the MCA and In-house data. Next, the standard workflow was run on integrated assay including scaling, dimension reduction and clustering using parameters described in https://satijalab.org/seurat/articles/integration_introduction.html #integration-goals-1. The clusters were then manually annotated using the RenameIdents function of Seurat 64.

### Var Gene Expression Per Cell

A set of 61 var genes were used for their average expression calculation. The var gene expression was calculated using Raw counts (RNA assay data slot). var gene Expression calculation=sum of var gene expression/sum of Expression of all genes.

### PfAP2-MRP Motif Identification

Sequences from the commonly identified peaks from two replicates were extracted from the P. falciparum 3D7 genome using the bedtools ‘getfasta’. These retrieved sequences were then uploaded to the DREME server68 to identify significantly enriched motifs in the peak region. Tomtom69 was used to compare the de novo identified motifs to previously in silico discovered motifs70.

### Immunoprecipitation of PfAP2-MRP and Identification of Proteins by Mass Spectrometry

To identify the AP2 interacting proteins, the on beads digestion with trypsin approach was used after immunoprecipitation of AP2 complex. Before performing the on beads digestion, after TE buffer washes the immunoprecipitated complex was washed two times with exchange buffer (100 mM NaCl, Tris 50 mM pH 7.5) for 10 minutes at 4° C. Subsequently, the beads were resuspended in 100 μl of 100 mM Triethylammonium bicarbonate (TEAB), and reduction of bound proteins was done with 1 mM DTT at 37° C. for 30 minutes on thermo mixture with constant shaking at 750 rpm. The sample was brought to room temperature and alkylated in the dark with 3 mM iodoacetamide for 45 minutes, the excess iodoacetamide was quenched with 3 mM DDT for 10 minutes (Sigma Aldrich). Afterward, the sample was digested with 2.5 μg trypsin (Promega) overnight on a thermo mixture with constant shaking at 1000 rpm. The resulting digested peptides were purified from beads, and trypsin digestion was stopped by adding TFA to a 2% final concentration. The acidified peptide was desalted using Sep-Pak C18 1 cc Vac Cartridge, 50 mg Sorbent per Cartridge (Waters). In brief, the Sep-Pak column was conditioned with 1 ml 100% methanol twice and equilibrated with 1 ml of 0.1% trifluoroacetic acid (TFA) twice. After that, the digested acidified peptides were loaded. The bound peptides were washed with 1 ml of 0.1% TFA twice and eluted with 300 μl of elution buffer (Acetonitrile 75% with 0.1% TFA in water) twice. The eluted peptides were dried in SpeedVac and kept at −80° C. till further use.

### LC-MS Analysis of Peptides

The LC-MS analysis was performed on Q-Exactive HF mass spectrometer coupled with an UltiMate™ 3000 UHPLC (Thermo Fisher Scientific). The peptides were dissolved in 0.1% formic acid (FA; Sigma Aldrich), and approximately 1 μg of peptides was separated on an Acclaim PepMap™ C18 column (75 μm I.D.×250 mm, 2 μm particle sizes, 100 Å pore sizes) with a gradient of 5-35% mobile phase A and B respectively for 55 minutes, ramping up to 90% phase B for 5 minutes, 90% phase B for 5 minutes and the column was conditioned to 2% phase B for 10 minutes with the flow rate of 300 nl/min⁻¹ (Phase A 0.1% FA, Phase B 99.9% ACN with 0.1% FA). The peptides were introduced into the mass spectrometer through Nanospray Flex with an electrospray potential of 2.5 kV. Data were acquired in the Orbitrap at the resolution of 60,000 in the mass range of 350-16000 m/z with a maximum ion accumulation time set to 50 ms. The 20 most intense ions with a threshold of more than 1×e⁶ and having the multiple charges were further fragmented by using higher-energy collision dissociation (HCD) at 15000 resolution. The Dynamic exclusion for HCD fragmentation was 30 seconds. The maximum time for fragmented ion accumulation was 30 ms, a target value of 2.50×e³, the normalized collision energy at 28%, and the isolation width of 1.6. During the acquisition, the ion transfer tube temperature was set at 160° C., data was acquired in data-dependent acquisition (DDA) mode, and the total run time was 75 minutes.

### Identification, Quantification and Statistical Analysis of LC-MS Data

Raw LC-MS data files from Q-Exactive HF were converted to .mgf files using Proteo Wizard MS covertgui 64 bit and analyzed using Mascot v2.4. The annotated protein sequence for Plasmodium falciparum: was downloaded from https://plasmodb.org (Release 51). Trypsin was set as the enzyme of choice with maximum missed cleavage 1, fixed modification carbamidomethyl (K), variable modification deamidated and oxidation of N, Q, and M, respectively, with the peptide and fragment mass tolerance at 0.6 Da.

ChIP qPCR

After de-crosslinking, ChIP DNA was purified using the Zymo ChIP DNA kit and quantified by Qubit HS DNA assay. The purified ChIP DNA was first diluted 20-fold in elution buffer and then analyzed by qPCR using the CFX-96 Biorad system. All ChIP primers used (Table 1) were first checked using genomic DNA to determine specificity (based on a single peak in the melting curve) and efficiency. ChIP qPCR data were analyzed using the ΔΔCt method. Pfap2-mrp ChIP-qPCR results are expressed as a percentage of input. Three biological replicates of both samples and negative control (mock IP using IgG) were used for ChIP-qPCR experiments.

### Flow Cytometry Using a Pooled Human Serum.

iRBCs with trophozoite stage parasites from cycle 1, treated with DMSO or rapamycin in cyle 0 to disrupt the first peak of PfAP2-MRP expression, were washed thrice with PBS supplemented with 0.1% BSA. iRBCS were either untreated or treated with Hiserum. When untreated, the same volume of 0.1% BSA in PBS was added and incubated for 30 mins at room temperature. Cells were washed thrice with 0.1% BSA in PBS, and all the samples were treated with sybr green (1×) and mouse anti-human IgG conjugated with Alexflour 647 (1:100 dilution, from BioLegend) for 30 mins at room temperature. After incubation, samples were washed thrice again with 0.1% BSA in PBS and analyzed on an BD LSR Fortessa flow cytometer (BD). Data were analyzed using FlowJo v9 software.

### In Situ Hi-C

Parasite were crosslinked using 1.25% formaldehyde in warm 1×PBS for 25 min at 37° C. with rotation. Glycine was then added to a final concentration of 150 mM to quench the formaldehyde and incubated for 15 min at 37° C. and 15 min at 4° C., both with rotation. Following centrifugation at 660×g for 15 min at 4° C., the pellet was resuspended in 5 volumes of ice-cold 1×PBS and incubated for 10 min at 4° C. with rotation. After another centrifugation at 660×g for 15 min at 4° C. the pellet was resuspended in 20 ml ice-cold 1×PBS. Several more washes in cold 1×PBS were used to clear cellular debris before resuspending in 1 ml 1×PBS and separated into multiple 1.5 ml tubes at a concentration of 1×10⁸ parasites per tube. The tubes were flash frozen in liquid nitrogen and stored at 80° C. before continuing with the rest of the in-situ Hi-C protocol⁷¹ using MboI restriction enzyme, with modifications to the standard protocol⁷². The final Hi-C libraries were sequenced using the Illumina NovaSeq 6000 using the S4 300 cycle flow cell for paired-end read libraries.

### Hi-C Data Processing and Analysis

Paired-end HiC library reads were processed using HiC-Pro (Servant et al., 2015) with default parameters and mapping at 10kb resolution to the P. falciparum genome (release-50, plasmodb.org). The ICED-normalized interaction matrices output by HiC-Pro were interaction counts-per-million normalized before generating interaction heatmaps. All intra-bin contacts and contacts within a two-bin distance were set to 0 to enhance visualization and the color map per chromosome was scaled based on the minimum number of interactions in the highest 10% of interacting bins to aid in comparison between samples. Interaction matrices for the replicates were merged and differential interactions were identified by calculating the log₂ fold change between the wild type and AP2-KO at each time point. Coordinate matrices generated by PASTIS⁷³ were visualized as 3D chromatin models using ChimeraX⁷⁴.

### Results

An Apicomplexan AP2 domain has been identified, containing DNA binding protein (referred to, herein as PfAP2-MRP: Master Regulator of Pathogenesis, Gene ID: PF3D7_1107800) that participate in repressing the majority of var genes, thus playing a crucial role in mutually exclusive expression pattern shown by var gene family members. PfAP2-MRP has two peaks of expression during the intra-erythrocytic developmental cycle at 16-hour post invasion (h.p.i.) and 40 h.p.i (FIG. 1A).

### PfAP2-MRP is Essential for Parasite Growth

363 genes have been identified with transcripts displaying a 24 hour (circadian-like) rhythmic periodicity in the 48-hour P. falciparum IDC, one of which was an ApiAP2 designated as PfAP2-MRP (FIG. 6A). PfAP2-MRP protein contains a single AP2 domain (residues 1487-1544, PFAM ID:PF00847), encoded in the second exon of PfAP2-MRP (FIG. 6B). PfAP2-MRP orthologs are present in the genus Plasmodium and in parasites from the class Coccidia but absent from the rest of the tree of life including other Apicomplexan species (FIG. 6C). The PfAP2-MRP and its P. berghei ortholog (PBANKA_0939100) were identified as essential during blood-stage development^{15, 16}. To investigate the function of PfAP2-MRP, a 3HA-tagged PfAP2-MRP inducible knockout P. falciparum (PfAP2-MRP-3HA:loxP) was generated using a parental line that expresses rapamycin (RAPA)-inducible dimerizable Cre recombinase (DiCre)13 (FIG. 1A, Methods). The entire second exon of PfAP2-MRP, including the sequence encoding the AP2 domain (as defined by PFAM ID:PF00847) and nuclear localization signal, was flanked with loxP sequences using CRISPR/Cas9 technology in a P. falciparum parental strain that contains rapamycin (RAPA)-inducible dimerizable Cre recombinase (DiCre) (Knuepfer et al., 2017) (FIG. 1A, also see methods). Immunofluorescence assays (IFA), confirmed the nuclear localization of PfAP2-P in various stages suggesting a basal level of PfAP2-P protein throughout the IDC.

Upon addition of RAPA to synchronized parasite culture, the loxP-flanked pfap2-mrp second exon was efficiently excised (FIG. 1B). A time-series RNA-seq analysis revealed that loxP flanked exon 2 deletion, and disruption of pfap2-mrp expression, occurred 14 to 16 hours after RAPA addition (FIG. 6G), allowing us to determine when to add RAPA to disrupt each peak of pfap2-mrp expression during the IDC. To ablate only the second peak of functional pfap2-mrp expression at 40 h.p.i. in the cycle 0, RAPA was added at ˜16 h.p.i in the same cycle (i.e., cycle 0). To ablate only the first peak of expression at 16 h.p.i. in cycle 1, RAPA was added at ˜35 h.p.i. in cycle 0, with parasite collection at 16 h.p.i. in cycle 1 (FIG. 6H). From here onwards, Δpfap2-mrp parasites (Pfap2-mrp-exon2 deleted) at 40 h.p.i. refers to parasites with disrupted second peak of expression that were collected at 40 h.p.i. and Δpfap2-mrp parasites at 16 h.p.i. refers to parasites with disrupted first peak of expression that were collected at 16 h.p.i.

RAPA addition at ˜16 h.p.i in cycle 0 ablated the second peak in the same cycle as demonstrated by RNA-seq (FIG. 1C) and western blot (FIG. 1D), and these Δpfap2-mrp parasites progressed through the IDC, forming morphologically normal segmented schizonts (49 h.p.i [cycle 0], FIG. 1E), with no significant difference in the number of nuclei in late schizonts of Δpfap2-mrp and DMSO (mock)-treated PfAP2-MRP-3HA:loxP parasites (FIG. 1F).

However, Δpfap2-mrp parasites failed to egress (FIG. 1E), with a resultant dramatic reduction in parasitemia in subsequent cycles compared to controls (FIG. 1G), indicating an essential role of PfAP2-MRP in parasite proliferation. In egress assays with highly synchronized segmented mature schizonts under either static or vigorous shaking (shear) conditions, no egress or increased parasitemia was observed for Δpfap2-mrp parasites, in contrast to the increased parasitemia in control cultures, irrespective of conditions (FIG. 1H); therefore, the blocked egress phenotype was not corrected by mechanical disruption of the iRBC.

To look for morphological differences, Δpfap2-mrp and control parasites were compared by indirect immunofluorescence with antibodies specific for proteins of the parasite surface pellicle: GAP45, a protein of the glideosome/inner membrane complex (IMC), and merozoite surface protein 7 (MSP7) (FIGS. 1I and 1J). In mock treated schizonts, both proteins were associated with the developing surface pellicle, enveloping individual nuclei in a characteristic pattern. However, in schizonts of rapamycin-treated cultures, neither protein was located at the periphery of merozoites, with a disordered appearance when compared to mock-treated parasites. These results indicate a failure to form morphologically intact fully mature merozoites in the absence of PfAP2-MRP, with development halted at a stage after nuclear division when the pellicular IMC and PPM would normally be distributed around individual nuclei.

This observation was further supported by electron microscopy of RAPA and compound 2 treated parasites (compound 2; c2 is a protein kinase G inhibitor which reversibly blocks parasite egress that results in stalled segmented schizonts with mature merozoites) at 49 h.p.i. (Thomas et al., 2018). In the control (compound 2 treated), the majority iRBCs contain mature schizonts with fully formed merozoites distributed around residual cytoplasmic mass containing the food vacuole with haemozoin crystals (FIG. 6D). The individual merozoites exhibited the apical polar ring, electron dense rhoptries and the nucleus enclosed by the pellicle (FIG. 6D, insert). In contrast, the vast majority of schizonts in the RAPA treated sample were frozen at the early segmenter stage (FIG. 6E). This was characterized by the initiation of merozoite formation around the periphery of the schizont (FIG. 6E). The apical end of the merozoite was initiated by the formation of cone-shaped inner membrane complex (IMC) beneath the plasmalemma (FIG. 6E, insert). However the posterior growth of the IMC appeared limited although there was evidence of the formation of the rhoptries and a nucleus associated with each daughter anlagen (FIG. 6E, insert). When quantified by counting a random sample of 50 iRBCs for each group, the control group showed 83% mature schizonts with 9% early segmenters and 8% dead parasites. In Δpfap2-mrp compared with control parasites, the distribution of these proteins was disordered. Electron microscopy analysis of Δpfap2-mrp and parasites treated with compound 2 (an inhibitor of egress17) at 49 h.p.i. revealed that most mature schizonts contained fully formed merozoites in the control sample. In contrast, most Δpfap2-mrp schizonts were at an earlier segmented stage. In samples of 50 iRBCs, the control group contained 83% mature schizonts compared to the Δpfap2-mrp group that contained only 2% mature schizonts (FIG. 6F). These results indicate that the essential function of PfAP2-MRP, if the gene is disrupted before second peak of expression, is at the very final stages of intra-erythrocytic development, prior to parasite egress from infected RBCs.

RAPA treatment from ˜35 h.p.i. had little effect on parasite egress or invasion at the end of cycle 0, but functional deletion of pfap2-mrp occurred well before the first expression peak in the next cycle. These parasites with the first peak of pfap2-mrp expression ablated were collected at 16 h.p.i. in cycle 1 (FIG. 6H), and loss of functional pfap2-mrp expression was confirmed by RNA-seq (FIG. 1N). Parasite development stalled at the late trophozoite/early schizont stage in cycle 1 (FIG. 1O), suggesting that the first peak of pfap2-mrp expression during the IDC plays a critical role in parasite development immediately after its expression at ˜16 h.p.i.

### PfAP2-MRP is a Crucial Regulator of Malaria Pathogenesis Associated Genes

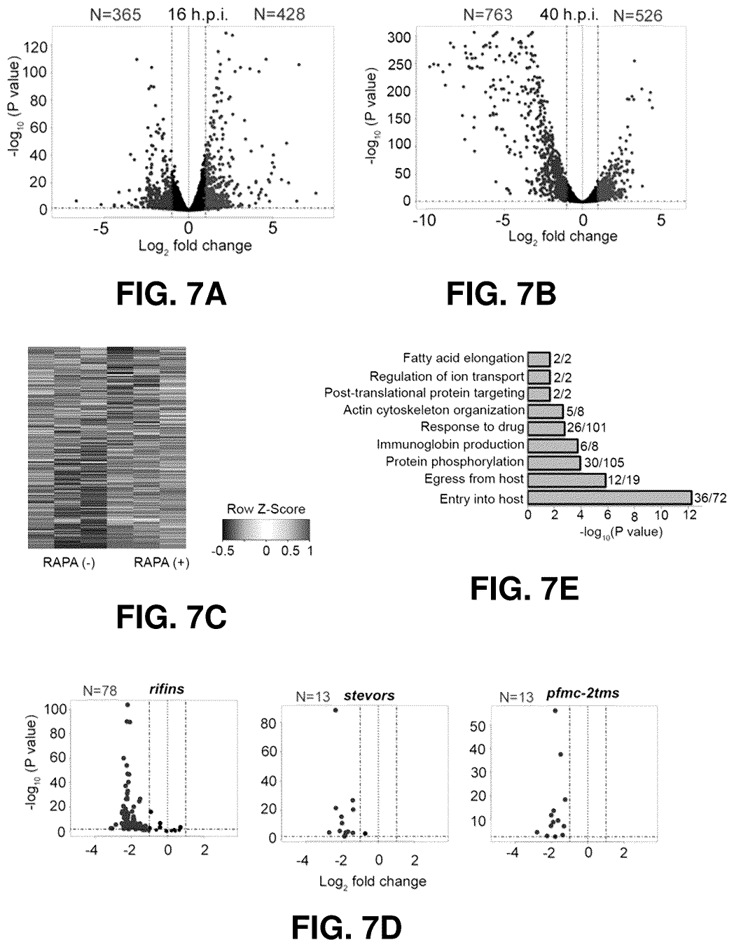

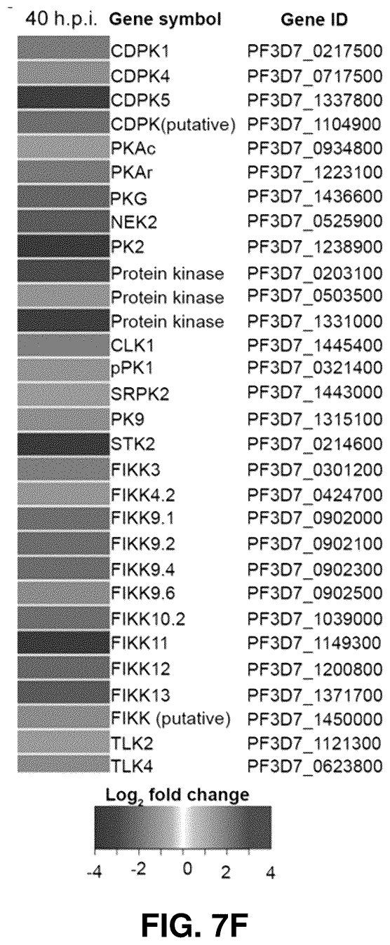

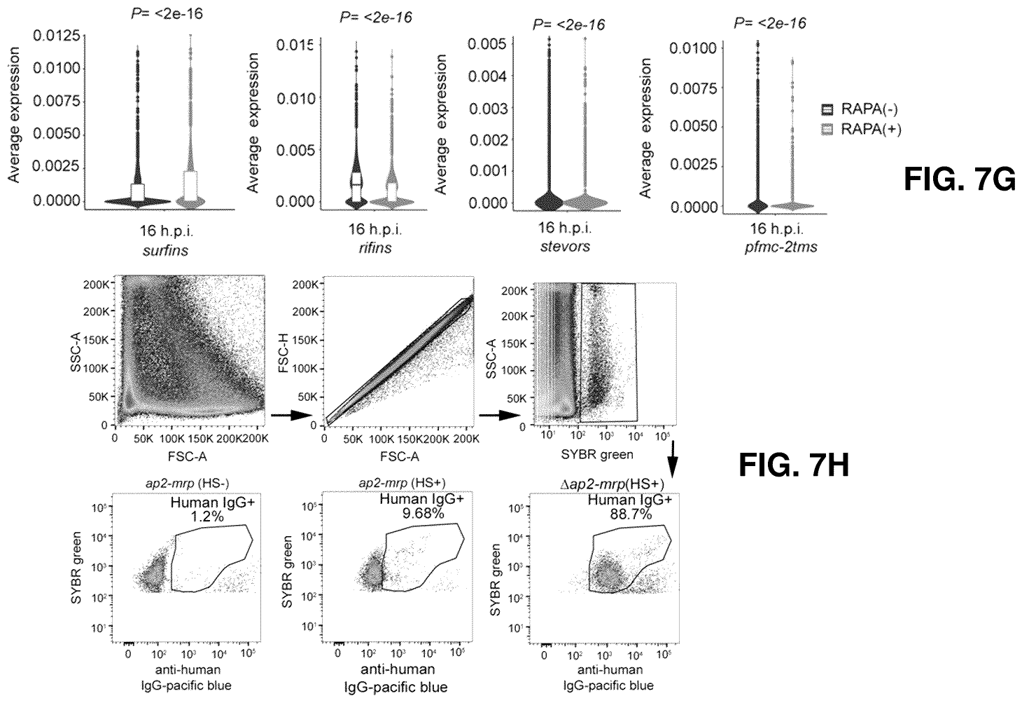

A comparative RNA-seq analysis of Δpfap2-mrp and control parasite populations at 16 and 40 h.p.i. identified 793 and 1,389 differentially expressed genes (FDR≤0.05), respectively (FIGS. 7A, 7B, and data not shown). Because disruption of the second peak of pfap2-mrp expression caused parasites to stall at the late segmented stage, studies examined whether the observed transcriptional differences at 40 h.p.i. were due to the knockout or reduced viability. Focus was placed on the expression of 1,042 genes that are known to have peak expression after 35 h.p.i.5, and it was expected that death or a delay in development of Δpfap2-mrp parasites would result in most of these genes being identified as down-regulated. However, 658 (63%) of them showed no significant change in expression level (FIG. 7C), indicating that observed differences in gene expression were due to pfap2-mrp deletion and not reduced viability.

The time-series RNA-seq data (FIG. 6E) showed that it takes 16-20 hours to delete the loxP flanked sequences from most parasites after RAPA treatment, with significant deletion in the last 2-3 hours. To test whether addition of RAPA significantly affected gene expression well before the transcriptome at 16 and 40 h.p.i. was analyzed, parasites were also collected for RNA-seq analysis at 8 h.p.i. and 30 h.p.i (stages at which pfap2-p had not reached maximum expression). A slight reduction of RNA-seq reads from exon 2 in RAPA-treated parasites was observed, compared to mock-treated parasites. Interestingly, only 89 genes were differentially expressed at 8 h.p.i. compared to 763 genes at 16 h.p.i., and 17 genes were differentially expressed at 30 h.p.i. compared to 1,389 genes at 40 h.p.i. These data suggest that RAPA addition has no significant effect on transcription even after 10-12 hours, possibly because pfap2-p truncation has not occurred yet in most parasites However, when the transcriptome was examined by 16 h.p.i. and 40 h.p.i. most parasites had lost the pfap2-p exon 2. Hence, the immediate effect of pfap2-p truncation on the parasite transcriptome at the two peaks of its expression was captured.

The P. falciparum genome contains 59 intact var genes and their expression is mutually exclusive: a single parasite expresses one var gene at a time, with the remainder of the family remaining transcriptionally silent by heterochromatin formation¹⁸. Gene ontology (GO) enrichment analysis of up-regulated genes in 16 and 40 h.p.i Δpfap2-mrp parasites identified pathogenesis as the most enriched biological term (adjusted P=1.23e-07 and 6.8e-10, respectively; Table 3 and data not shown). The var gene family has the largest number of members assigned to this GO term, with significant up-regulation of 24 and 29 var genes (Padj<0.05) in Δpfap2-mrp compared to control parasites at 16 h.p.i. and 40 h.p.i., respectively (FIGS. 2A and 2B). Data was validated by quantitative real time PCR (qRT-PCR) (FIGS. 2C and 2D). These results suggest that PfAP2-MRP may acts a repressor of most var genes.

TABLE 2
Downregulated Genes at 40 h.p.i

| | Genes in the | Genes in your | Percent of | | | | | |
|---|---|---|---|---|---|---|---|---|
| | bkgd with | result with | bkgd genes in | Fold | Odds | | | |
| GO Term | this term | this term | your result | enrichment | ratio | P-value | Benjamini | Bonferroni |
| entry into host | 72 | 36 | 50 | 3.47 | 6.23 | 5.56E−13 | 2.29E−10 | 4.59E−10 |
| exit from host | 19 | 12 | 63.2 | 4.38 | 10.36 | 1.39E−06 | 1.43E−04 | 1.15E−03 |
| protein | 105 | 30 | 28.6 | 1.98 | 2.44 | 1.09E−04 | 9.02E−03 | 9.02E−02 |
| phosphorylation | | | | | | | | |
| immunoglobulin | 8 | 6 | 75 | 5.21 | 17.98 | 1.89E−04 | 1.30E−02 | 1.56E−01 |
| production | | | | | | | | |
| response to drug | 101 | 26 | 25.7 | 1.79 | 2.1 | 1.73E−03 | 7.53E−02 | 1.00E+00 |
| actin | 8 | 5 | 62.5 | 4.34 | 9.97 | 2.35E−03 | 9.23E−02 | 1.00E+00 |
| cytoskeleton | | | | | | | | |
| organization | | | | | | | | |
| posttranslational | 2 | 2 | 100 | 6.94 | Infinity | 2.07E−02 | 4.62E−01 | 1.00E+00 |
| protein targeting | | | | | | | | |
| to membrane, | | | | | | | | |
| translocation | | | | | | | | |
| regulation of ion | 2 | 2 | 100 | 6.94 | Infinity | 2.07E−02 | 4.62E−01 | 1.00E+00 |
| transport | | | | | | | | |
| fatty acid | 2 | 2 | 100 | 6.94 | Infinity | 2.07E−02 | 4.62E−01 | 1.00E+00 |
| elongation | | | | | | | | |

TABLE 3
Upregulated Genes at 40 h.p.i

| | Genes in the | Genes in your | Percent of | | | | | |
|---|---|---|---|---|---|---|---|---|
| | bkgd with | result with this | bkgd genes in | Fold | Odds | | | |
| GO Term | this term | term | your result | enrichment | ratio | P-value | Benjamini | Bonferroni |
| pathogenesis | 100 | 34 | 34 | 3.77 | 5.59 | 1.71E−12 | 6.80E−10 | 9.75E−10 |
| fatty acid | 36 | 15 | 41.7 | 4.63 | 7.45 | 1.54E−07 | 1.75E−05 | 8.75E−05 |
| metabolic | | | | | | | | |
| process | | | | | | | | |
| acetyl-CoA | 4 | 4 | 100 | 11.1 | Infinity | 6.50E−05 | 2.88E−03 | 3.70E−02 |
| biosynthetic | | | | | | | | |
| process | | | | | | | | |
| carboxylic acid | 55 | 15 | 27.3 | 3.03 | 3.89 | 6.59E−05 | 2.88E−03 | 3.75E−02 |
| biosynthetic | | | | | | | | |
| process | | | | | | | | |
| intracellular | 13 | 6 | 46.2 | 5.12 | 8.77 | 5.11E−04 | 1.91E−02 | 2.91E−01 |
| protein | | | | | | | | |
| transmembrane | | | | | | | | |
| transport | | | | | | | | |
| lipid | 67 | 15 | 22.4 | 2.49 | 2.99 | 6.97E−04 | 1.91E−02 | 3.97E−01 |
| biosynthetic | | | | | | | | |
| process | | | | | | | | |
| mitochondrial | 9 | 4 | 44.4 | 4.93 | 8.15 | 5.67E−03 | 8.74E−02 | 1.00E+00 |
| transmembrane | | | | | | | | |
| transport | | | | | | | | |
| ribonucleoside | 9 | 4 | 44.4 | 4.93 | 8.15 | 5.67E−03 | 8.74E−02 | 1.00E+00 |
| bisphosphate | | | | | | | | |
| biosynthetic | | | | | | | | |
| process | | | | | | | | |
| organelle fusion | 3 | 2 | 66.7 | 7.4 | 20.3 | 2.28E−02 | 2.55E−01 | 1.00E+00 |
| movement of | 48 | 9 | 18.8 | 2.08 | 2.36 | 2.53E−02 | 2.66E−01 | 1.00E+00 |
| cell or | | | | | | | | |
| subcellular | | | | | | | | |
| component | | | | | | | | |
| cofactor | 60 | 10 | 16.7 | 1.85 | 2.05 | 3.98E−02 | 3.60E−01 | 1.00E+00 |
| biosynthetic | | | | | | | | |
| process | | | | | | | | |

Also observed was the up-regulation of 4 of 8 surfins at 16 h.p.i. (Data not shown). Several gene families coding for other variant proteins such as rifins (n=78/132), stevors (n=13/30), and Pfmc-2 ms (n=13/13) were significantly down-regulated (FDR≤0.05) at 16 h.p.i., suggesting that these genes are positively regulated by PfAP2-MRP (FIG. 7D, Data not shown). While some members of the hyp and phist gene families, encoding exportome-associated proteins showed up-regulation, others showed down-regulation at 16 h.p.i., consistent with a functional diversification (Data not shown). Together, these results suggest that pfap2-mrp expression at 16 h.p.i. has a major role in regulating the expression of genes coding for proteins important in antigenic variation and host cell remodeling.

Gene ontology (GO) enrichment analysis of the down-regulated genes in Δpfap2-mrp parasites at 40 h.p.i. identified entry into and egress from the host cell as the two most enriched biological process terms (FIG. 7E, Table 2), consistent with the finding that deletion of pfap2-mrp critically affects late merozoite development and egress. Of the top twenty down-regulated genes, fifteen function in parasite egress or invasion (FIG. 2E). The expression of all top down-regulated genes peaks just after the second peak of pfap2-mrp expression (FIG. 6A), consistent with the proposed role of PfAP2-MRP in controlling their expression. For example, known egress-associated genes coding for SUB1-2 and -3, SERA5 and -6, Plasmepsin X, CDPK1 and −5, MSA 180, phospholipase, PKG, and MSP1 were down-regulated (Data not shown). Strikingly, 36 out of 72 known invasion-associated genes, including those coding for micronemal, rhoptry, merozoite surface, IMC and motor proteins were also down-regulated (FIG. 2F, Data not shown). Western blots and immunofluorescence assays were used to confirm the reduced abundance for a few selected proteins (FIG. 2G). Altogether, the data suggest that PfAP2-MRP is essential to the expression of egress and invasion-associated genes.

Other biological processes enriched in the group of down-regulated genes in Δpfap2-mrp parasites at 40 h.p.i. include protein phosphorylation, actin cytoskeleton organization, and fatty acid elongation. Protein phosphorylation has a significant role in control of both egress and invasion in addition to other cell cycle events^{19,20}. 30 out of 105 genes annotated were identified as involved in protein phosphorylation to be significantly down-regulated (FIG. 7F, Table 2), including 10 out of 21 FIKK kinase genes, the majority of which (17) are exported^{21, 22}. This suggests a broad regulatory role for PfAP2-MRP in signaling processes during egress and invasion

### PfAP2-MRP is Essential for Trophozoite Stage Development

Subsequent studies tested whether the gene expression pattern observed in the bulk RNA-seq data is also observed at single cell resolution. Single-cell RNA sequencing (scRNA-seq) was performed using parasites from 16 h.p.i. (control parasites and parasites with the first peak of expression disrupted) and 40 h.p.i. (control parasites and parasites with only the second peak of expression disrupted) stages. Based on the stage-specific annotation of cells in the malaria cell atlas (MCA)²³, 16 h.p.i. cells are ring/early trophozoite parasites and 40 h.p.i. cells are late trophozoite and schizont stage parasites (FIG. 2H). It was observed that more single Δpfap2-mrp parasites with disrupted first peak of expression at 16 h.p.i. were classified as ring rather than trophozoite compared to controls (FIG. 2I). However, no difference in stage was observed at 40 h.p.i. for parasites with disrupted second peak of expression suggesting that it is not RAPA toxicity that is responsible for the observed delay of Δpfap2-mrp parasite development at 16 h.p.i. The delay observed in the parasite development may be an early phenotype resulting from the loss of the first pfap2-mrp expression peak (FIG. 1N), which suggest that the first peak of expression is critical for trophozoite development. No difference in developmental stages between control and Δpfap2-mrp parasites with only disrupted second peak of expression at 40 h.p.i. further suggests that differentially expressed genes at 40 h.p.i. (FIG. 7B) were due to pfap2-mrp deletion and not due to difference in developmental stage or reduced viability.

The Role of PfAP2-MRP in Repressing Var Genes Confirmed with Single Cells

From the scRNA-seq analysis, observed were significantly higher expression of vars (FIG. 2J) and surfins (FIG. 7G) and down-regulation of rifins and pfmc-2tms in Δpfap2-mrp parasites, supporting the bulk RNA-seq data (FIG. 7G). Other studies examined whether pfap2-mrp deletion activated expression of multiple var genes in a single cell. Most control parasites expressed only one var gene following the mutually exclusive expression pattern reported for var genes¹⁸, but a few expressed two or more var genes (FIG. 2K). A significantly higher number of Δpfap2-mrp parasites expressed two or more var genes (P=6.23e-13, Fisher's exact test, FIG. 2K), indicating apparent disruption of mutually exclusive var gene expression in the Δpfap2-mrp parasites.

### ΔPfap2-Mrp Parasites Expresses Multiple Surface PfEMP1

To determine whether in Δpfap2-mrp parasites expressing multiple var genes, EMP1s are translated and exported to the iRBC surface, a FACS-based assay was developed with pooled serum from patients infected with P. falciparum²⁴. Amongst many exported malaria proteins, PfEMP1 is a major target of naturally acquired antibodies²⁵, and therefore it was hypothesized that if Δpfap2-mrp parasites express multiple PfEMPls, then they will bind more antibodies in the serum against different PfEMP1s, compared to the control parasites. Significantly more Δpfap2-mrp iRBCs than control parasites with bound antibody were observed(P<0.0001, two-tailed t-test, FIG. 2L, FIG. 7H). This result indicates that activation of multiple var genes and the translation and transport of PfEMP1 to the iRBC surface occurs in Δpfap2-mrp parasites, and suggests that PfAP2-MRP is involved broadly in silencing var gene expression.

### PfAP2-MRP is a Repressor of Early Gametocyte-Associated Marker Genes

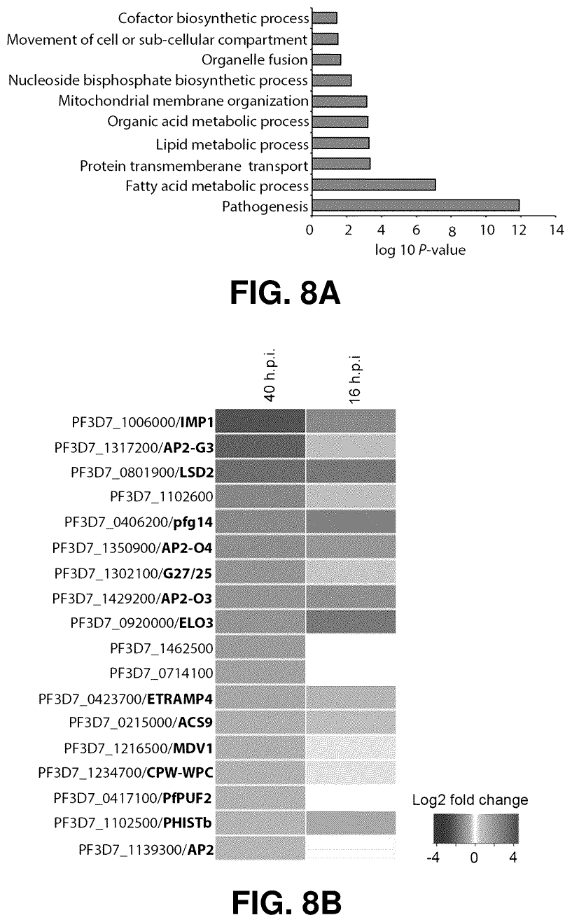

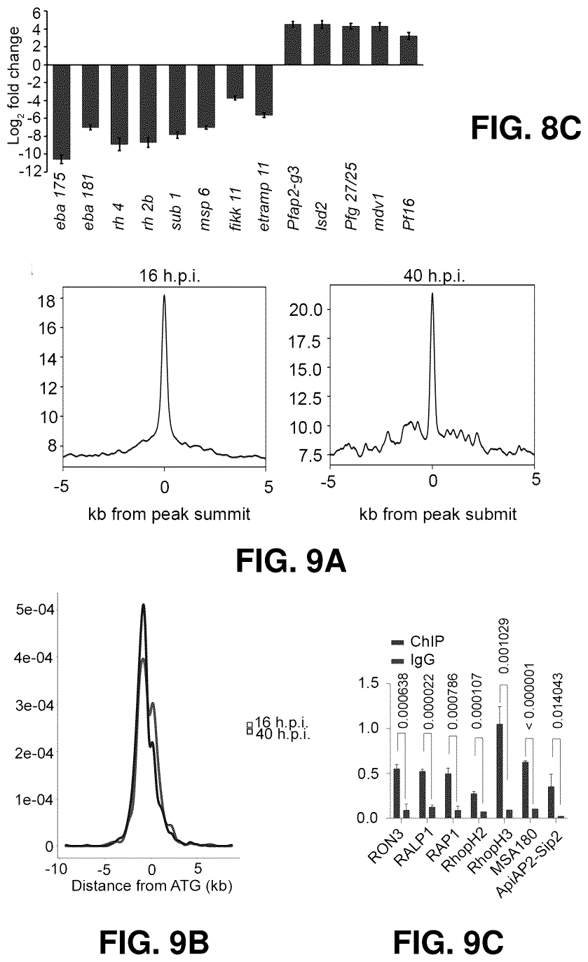

GO enrichment analysis of 526 up-regulated genes at 40 h.p.i. in Δpfap2-mrp schizonts identified significant enrichment of many biological processes, including lipid and fatty acid metabolism (FIG. 8A and Table 3). Genes such as elongation of fatty acids protein 3 (elo3) and acyl-CoA synthetase 9 (acs9) have essential roles in gametocyte development^{16,26} and were up-regulated in 16 and 40 h.p.i. Δpfap2-mrp parasites (Data not shown). The list of up-regulated genes was examined for other known or putative early gametocyte marker genes^{27,28} and 18 out of 28 genes were found to be up-regulated in both 16 and 40 h.p.i. Δpfap2-mrp parasites (FIG. 8B, Data not shown), suggesting that PfAP2-MRP is also a repressor of commitment to sexual stage development. The expression of some differentially regulated genes were validated by qRT-PCR, and found was strong agreement between RNA-seq and qRT-PCR data (FIG. 8C).

### PfAP2-MRP is a Direct Regulator of Heterochromatin-Associated Genes

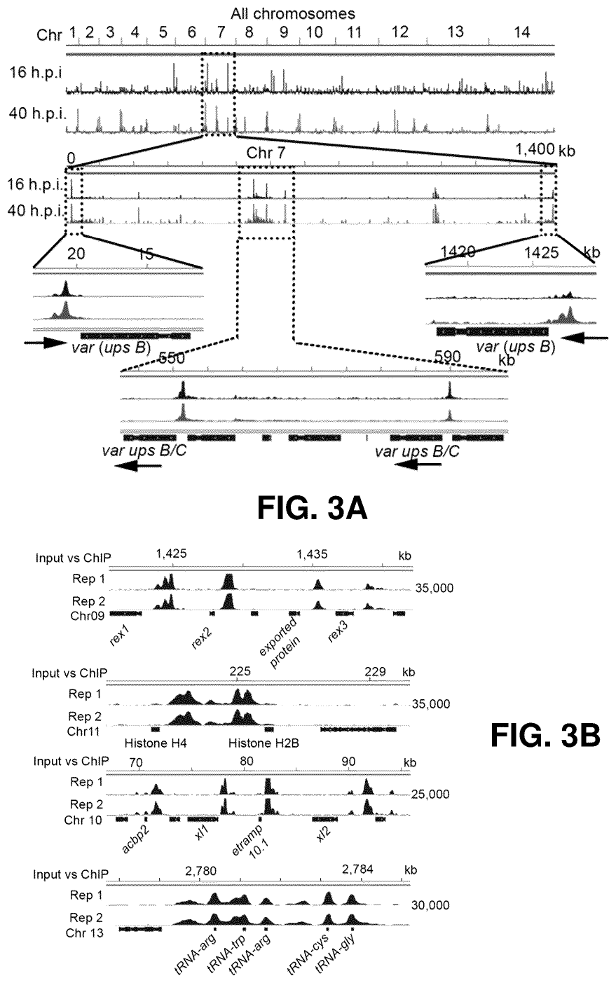

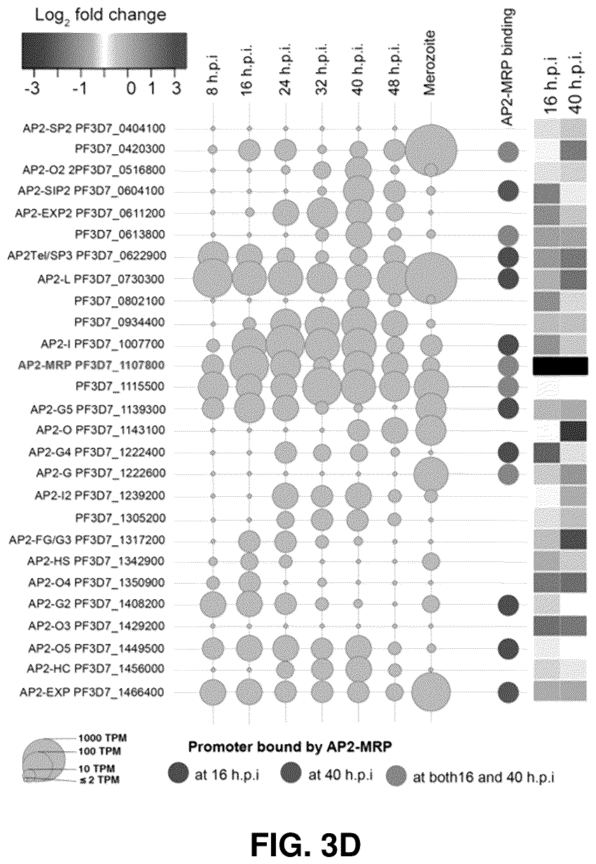

To distinguish between direct and indirect targets of PfAP2-MRP, chromatin immunoprecipitation with the 3HA-epitope tagged PfAP2-MRP protein followed by sequencing (ChIP-seq) was performed. Identified were 1,081 and 640 ChIP-seq peaks at 16 and 40 h.p.i., respectively, of which 78% and 68% were in intergenic/promoter regions upstream of at least one gene (with most located less than 2 kb upstream of an ATG translational start site) (FIGS. 9A and 9B, and data not shown). A few PfAP2-MRP binding regions identified by ChIP-seq at 40 h.p.i. were validated using ChIP-qPCR, with complete agreement (FIG. 9C). Consistent with previous finding29, binding of PfAP2-MRP to both central and sub-telomeric heterochromatin regions at both 16 and 40 h.p.i. stages was observed, in addition to its binding to the promoter of euchromatic genes (FIG. 9D). GO analysis identified encoded proteins enriched in several processes, the most significant of which is antigenic variation (Padj=8.36e-32 at 16 h.p.i and 6.42e-38 at 40 h.p.i.; Data not shown). It was observed that PfAP2-MRP binds significantly (q<0.05) to the promoters of at least 37 and 33 var genes at 16 h.p.i. and 40 h.p.i, respectively; a total of 45 var genes, including both sub-telomeric and internal var genes of all the upstream sequence (ups) types (FIG. 3A and FIG. 9E).

PfAP2-MRP binding at both 16 and 40 h.p.i. was also enriched at the promoter of genes encoding exportome proteins with relatively strong signals at 16 h.p.i stage and at the promoter or intragenic regions of many other gene families for antigenic variant proteins (rifins, stevor, surfins and pfmc-2tms) with relatively weaker yet statistically significant signals at 16 h.p.i. stage (FIGS. 3B and 3C, and data not shown)). Most of these genes were also differentially expressed in Δpfap2-mrp parasites suggesting that they are under direct PfAP2-MRP control (Data not shown). These gene families for antigenic variant and other exported proteins are located within the chromosomal central and subtelomeric heterochromatin regions.

The promoters of genes associated with critical processes in early-stage development such as translation (Padj=1.3e-10), glycolytic process (Padj=0.0005) and nucleosome assembly (Padj=0.02) were also strongly bound by PfAP2-MRP at 16 h.p.i. (FIG. 3C, and data not shown)) suggesting these processes are regulated by PfAP2-MRP, and to some extent this may explain why development of parasites with disrupted first peak of expression stalled at late trophozoite stage. The data herein was compared with published ChIP-seq data for PfAP2-P captured at three different stages (i.e. ring, trophozoite and schizont). ChIP-seq peaks identified at the 16 h.p.i stage had the highest percentage of peaks from ring stage parasites (47% of 16 h.p.i. peaks) with shared genomic regions, and ChIP-seq peaks from 40 h.p.i. stage had the highest percentage of peaks from trophozoite stages (41% of 40 h.p.i. peaks) with shared genomic regions. Most peaks shared between 16 h.p.i. and ring stage parasites and 40 h.p.i. and trophozoite stages correspond to genes coding for biological processes such as antigenic variation, translation, glycolytic process, nucleosome assembly; and genes coding for exportome proteins, AP2 transcription factors and invasion.

### PfAP2-MRP Binds to its Own Promoter and Those of Another 13 ApiAP2s

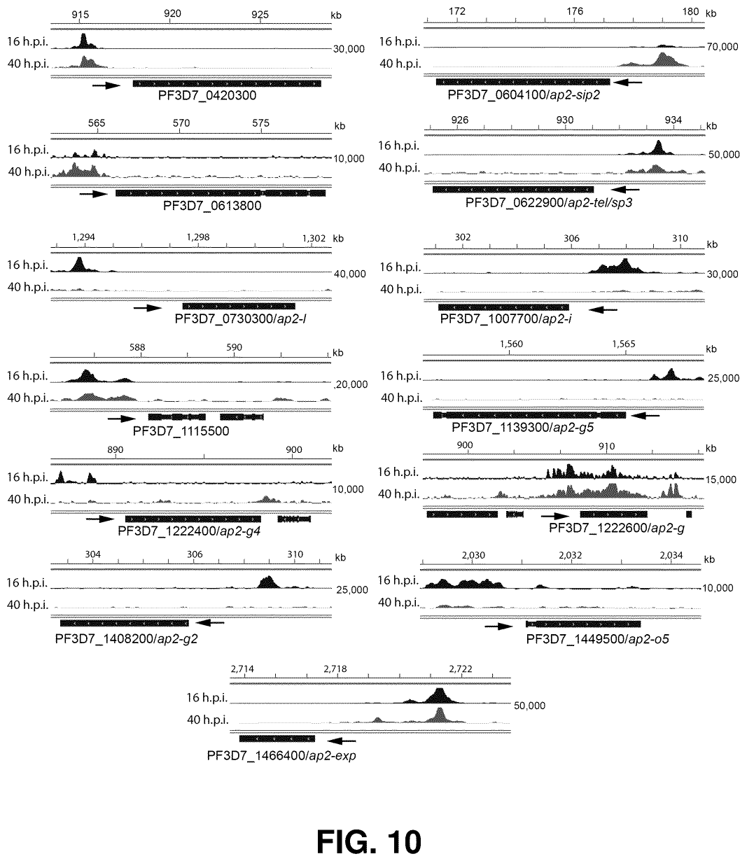

PfAP2-MRP binds to the promoter of a total of 14 Pfapiap2 genes including its own promoter at both 16 h.p.i. and 40 h.p.i. (FIG. 3D, FIG. 10 and data not shown)). Nine of these 13 apiap2s were differentially expressed in Δpfap2-mrp parasites at one or both of the 16 and 40 h.p.i. time points, suggesting that they are under the direct control of PfAP2-MRP.

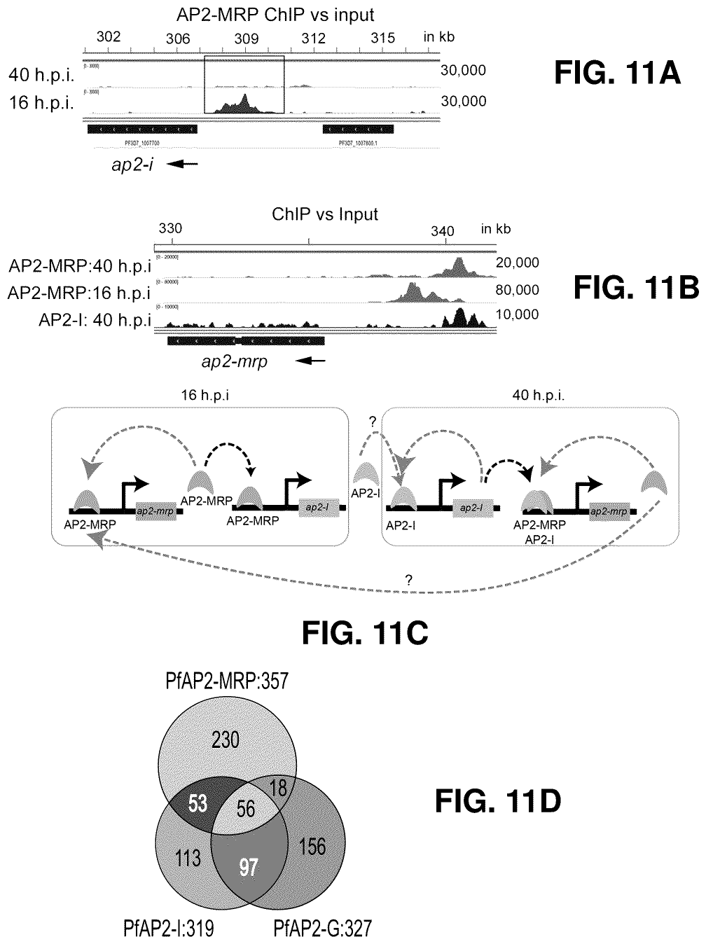

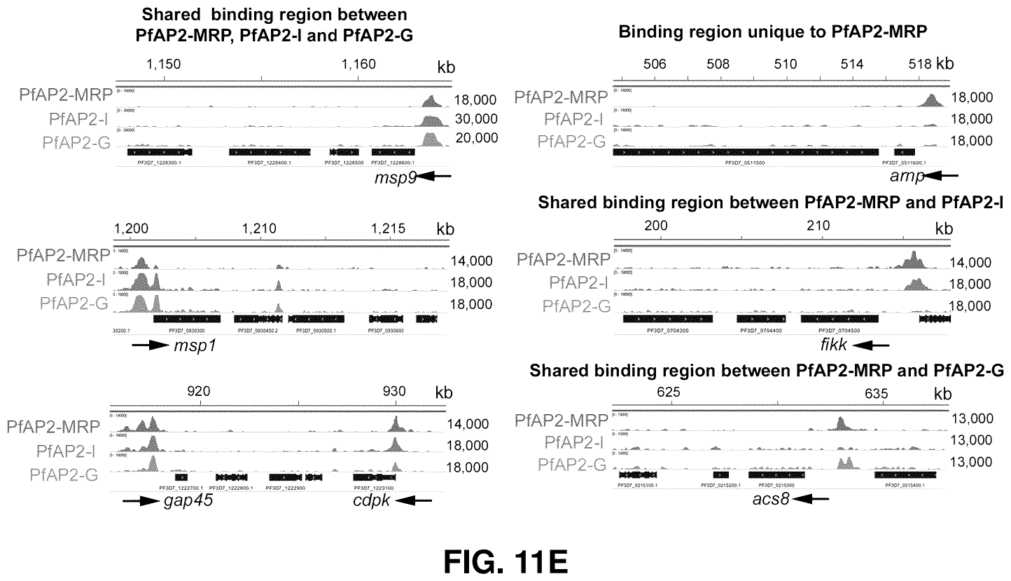

Binding of PfAP2-MRP to the pfap2-i promoter was observed (FIG. 11A), and down-regulation of pfap2-i in Δpfap2-mrp parasites at 16 h.p.i. (FIG. 3D). It has been reported that PfAP2-I binds the pfap2-mrp and its own promoter at 40 h.p.i.30, and in this study, it was observed that PfAP2-MRP binds to its own promoter at both 16 h.p.i. and 40 h.p.i. (FIG. 11B). Together, this information suggests that PfAP2-MRP autoregulates its expression at both 16 h.p.i. and 40 h.p.i., positively regulates the expression of pfap2-i at 16 h.p.i.; PfAP2-I autoregulates its expression at 40 h.p.i. and pfap2-mrp expression might be additionally controlled by PfAP2-I at ˜40 h.p.i (combinatorial regulation; FIG. 11C).

PfAP2-MRP bound to the same promoter region of many genes as PfAP2-I and PfAP2-G, two proteins implicated in invasion³⁰ and gametocytogenesis³¹, respectively (FIGS. 11D and 11E, and data not shown)), suggesting a complex interplay of ApiAP2 DNA-binding proteins in control of gene expression. Genes with promoters bound by PfAP2-MRP, PfAP2-I, and PfAP2-G include gap45; msp1 and -9; dblmsp; etramp 4-10.2 and Pfap2-g (FIG. 11E).

### PfAP2-MRP is an Indirect Regulator of Most Invasion- and Egress-Associated Genes

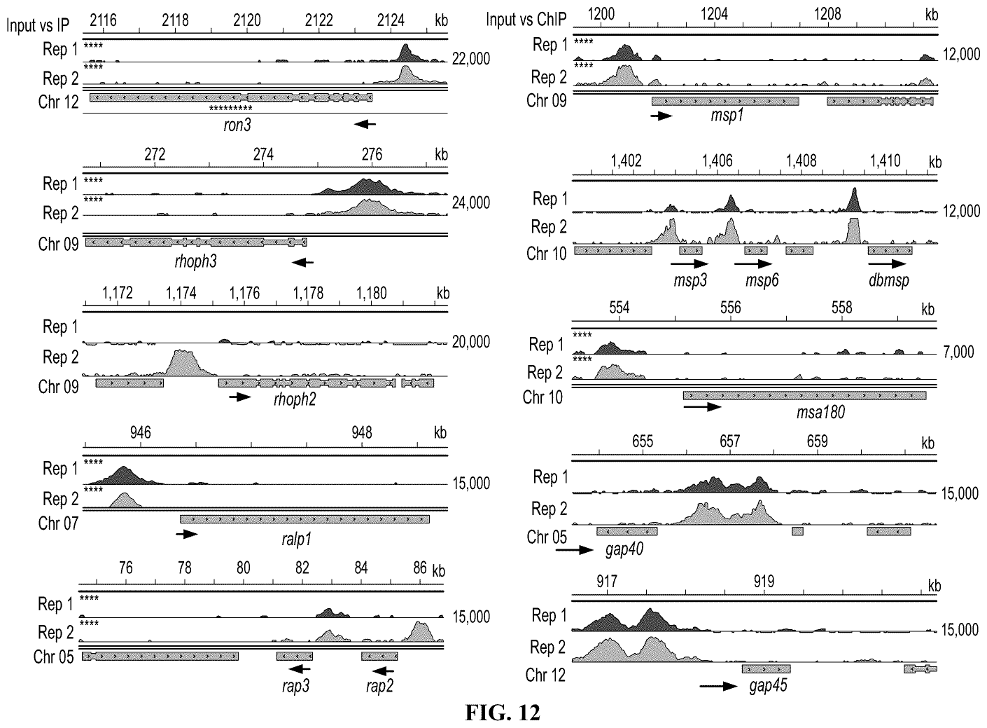

PfAP2-MRP binds to the promoters of only few invasion- and egress-associated genes at 40 h.p.i. (FIG. 12 and data not shown)). These genes were significantly (FDR≤0.05; log 2 fold change≤−1) or weakly (log 2 fold change between −0.5 to −1) down-regulated in Δpfap2-mrp parasites at 40 h.p.i. compared to controls (Data not shown), suggesting that PfAP2-MRP directly regulates these genes. However, PfAP2-MRP was not bound to the promoters of most top down-regulated invasion- and egress-associated genes (FIG. 2B, Data not shown) except for msa180, zinc finger protein (PF3D7_0818100), sub1 and msp6 (data not shown)). These results suggest that the consequences of PfAP2-MRP exon 2 deletion are both direct and indirect on gene expression.

### PfAP2-MRP Binds to Conserved Sequence Motifs

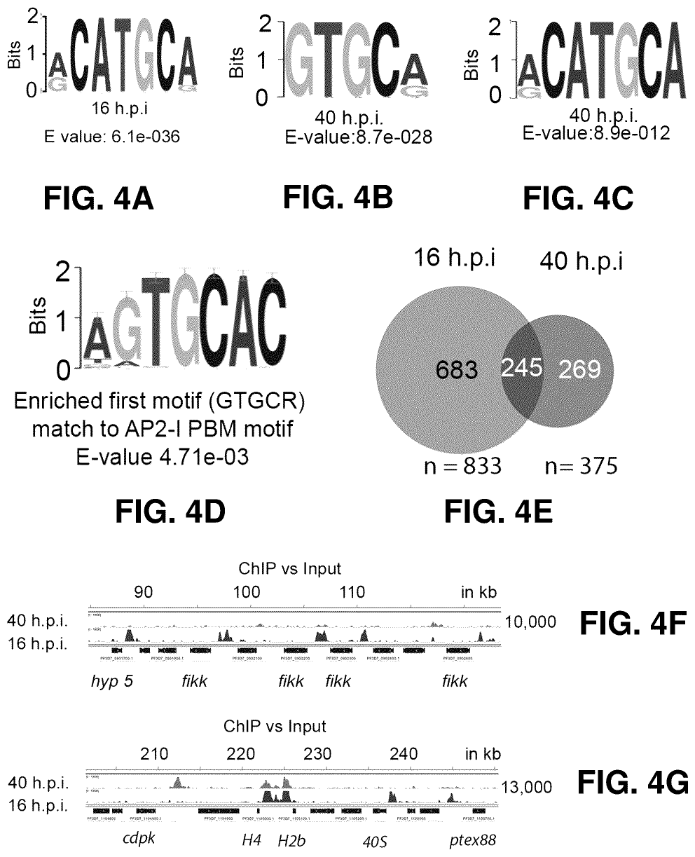

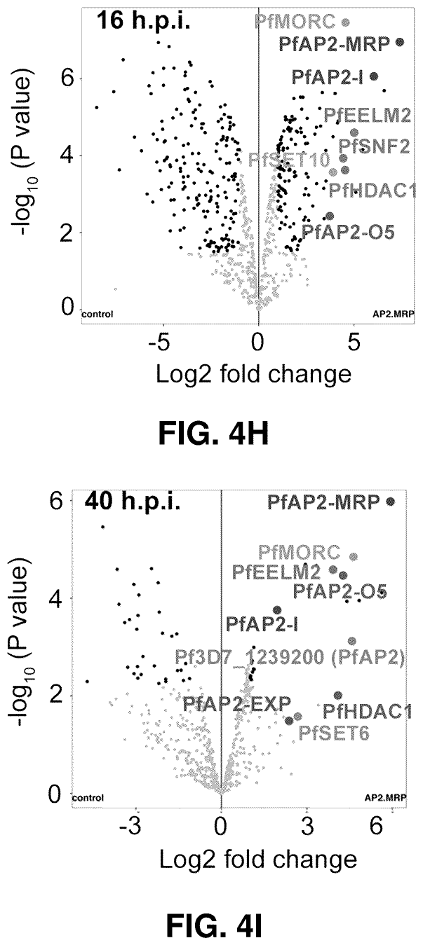

Analysis of the sequences bound by PfAP2-MRP at 16 h.p.i. and at 40 h.p.i. identified RCATGCR (6.1e-36, FIG. 4A); and GTGCR (8.7e-28, FIG. 4B) and RCATGCA (8.9e-12, FIG. 4C) as the most significantly enriched motifs respectively, excluding highly degenerate motifs. GTGCR is very similar to the motif bound by PfAP2-I in vitro³⁰ (P=4.7e-03, FIG. 4D), consistent with the observation that PfAP2-MRP binds many sequences bound by PfAP2-I. RCATGCR (identified at 16 h.p.i.) is very similar to RCATGCA (identified at 40 h.p.i., P=6.97e-5, FIG. 4C) suggesting that PfAP2-MRP binding is sequence-specific with dynamic binding to similar sequence motifs in different genomic regions at different IDC stages (FIGS. 4E-4G and data not shown). Such differential binding might be due to differences in PfAP2-MRP accessibility to different genomic regions, such as that conferred by differential chromatin structures.

PfAP2-MRP Associates with Known and Putative Epigenetic Regulators

Since PfAP2-MRP activates or represses the expression of most gene families under epigenetic control and coding for clonally variant antigenic proteins, the hypothesis was that PfAP2-MRP recruits epigenetic regulator(s) to modulate target genes. In support of this hypothesis, immunoprecipitation and mass spectrometry of PfAP2-MRP-3HA protein complexes identified several known or putative histone modifiers and chromatin remodelers such as a putative microrchdia (MORC) family protein, multiple EELM2 domain-containing proteins, imitation protein (ISW1), histone deacetylase 1 (HDAC1), chromodomain-helicase-DNA-binding (CHD1) proteins, together with five other PfAP2 DNA binding proteins (FIGS. 4H and 4I and data not shown). While PfAP2MRP-associated histone modifiers such as PfSET10, PfSET6, and chromatin remodeler PfISWI proteins are transcriptional activators³²⁻³⁴, PfMORC-a putative chromatin remodeler along with PfHDAC1-a histone deacetylase are associated with repressed genes and are regarded as transcriptional repressors³⁵⁻³⁶. Many of the proteins associated with PfAP2-MRP were present at both 16 and 40 h.p.i., but others were unique to one time point. The association of PfAP2-I, PfAP2-MRP and ISWI30 is consistent with the observed overlap between PfAP2-MRP and PfAP2-I binding sites and the significant similarity between the enriched motifs (data not shown).

Depletion of PfAP2-P does not Alter Known Histone Modifications in Heterochromatic Regions

Additional studies analyzed the state of two histone modifications (i.e. H3K9ac and H3K4me3) that are known to enrich at the active var gene promoter and one histone modification H3K9me3 known to associate with repressed var genes (Heterochromatin protein 1 mediated repression). The heterochromatic regions were enriched with the H3K9me3 mark and depleted of H3K9ac and H3K4me3 marks in mock-treated parasites supporting our ChIP data. Clear overlaps between H3K9me3 marks and PfAP2-P occupancy could be seen in the heterochromatic regions of the genome (data not shown). However, there was either a slight or no depletion of the H3K9me3 mark in heterochromatic regions including within the body of var genes in the pfap2-p truncated parasites compared to wild-type at 16 and 40 h.p.i., respectively (data not shown). Similarly, no enrichment of gene activation marks H3K9ac and H3K4me3 was observed in the known heterochromatic regions of the genome including in var promoters (data not shown). These results suggest an alternative mode of var gene regulation involving novel pfap2-p associated chromatin remodelers and histone modifiers that are independent of the histone marks tested in this study (i.e. H3K9me3, H3K9ac and H3K4me3).

### Depletion of PfAP2-MRP Increases Chromatin Accessibility

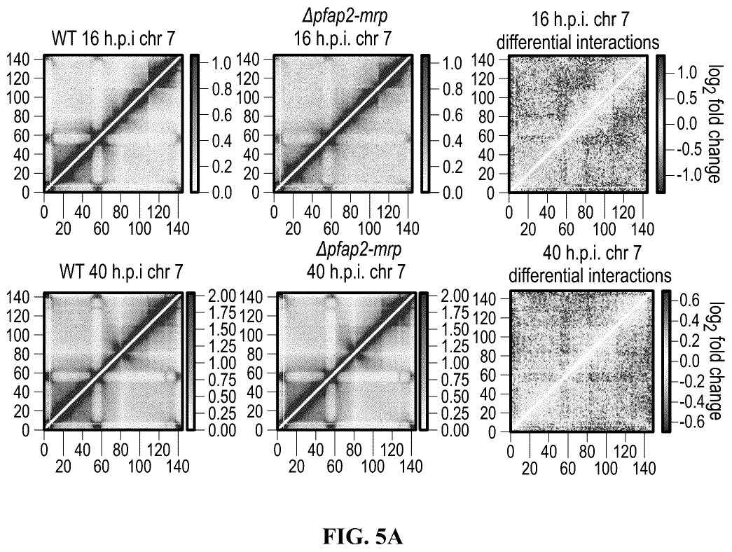

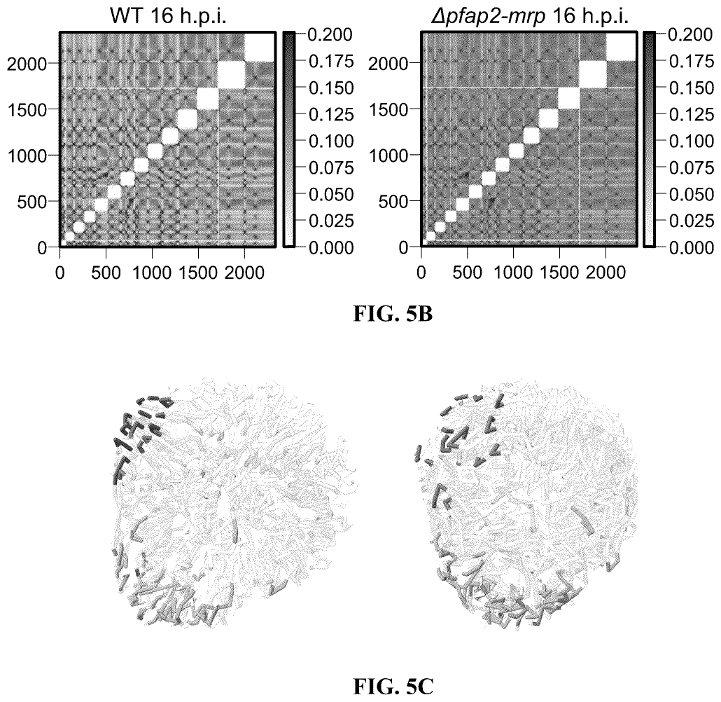

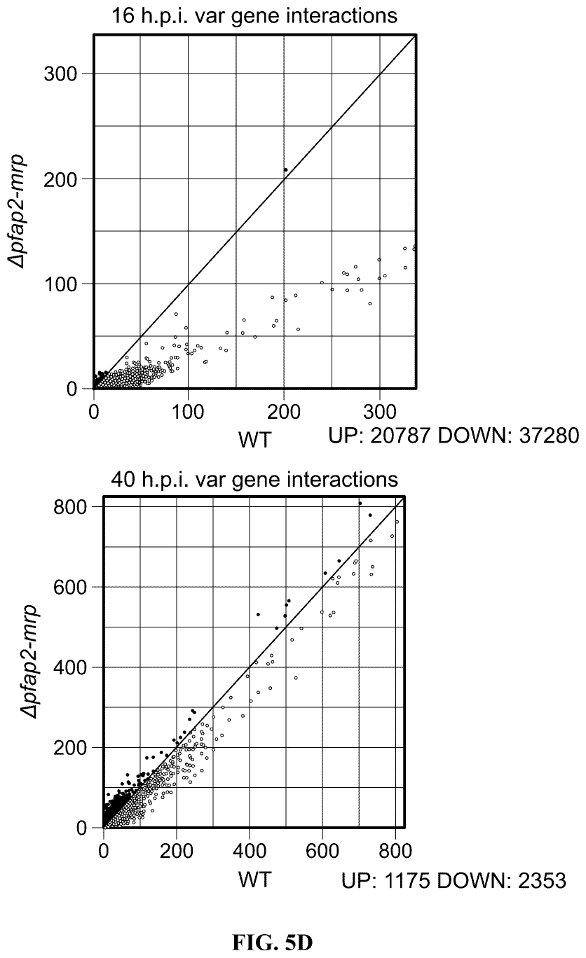

While the relationship between chromatin organization and gene regulation remains unclear, parasite chromatin is organized into euchromatin and heterochromatin clusters and can act as a scaffold facilitating gene expression³⁷⁻³⁹. Furthermore, considering the strong effect of disruption of pfap2-mrp on genes associated with heterochromatin regions, studies explored the effect of pfap2-mrp deletion on chromatin structure. Comparative analysis was performed on the chromatin conformation landscape using whole-genome chromosome conformation capture (Hi-C) followed by deep sequencing from either the tagged or the Δpfap2-mrp strains at 16 and 40 h.p.i. Correlation analysis of Hi-C replicates indicated a clear reproducibility at 16 and 40 h.p.i. where overall intra-chromosomal and interchromosomal interaction matrices appeared largely unchanged for each strain (FIG. 5A, and data not shown). However, comparative analysis between the tagged and the Δpfap2-mrp lines revealed a slight reduction compared to background in long distance interactions and heterochromatin clusters in Δpfap2-mrp (FIG. 5B, and data not shown) at 16 h.p.i. The effect was detected more globally at 40 h.p.i. Genome-wide mapping of interaction changes revealed a reduction in interaction frequency between telomere ends including internal var genes (FIG. 5C), consistent with a reduced clustering of the heterochromatin. Further support was provided by genome-wide 3D modeling on these two strains wherein clear separation of telomeric clustering was seen in Δpfap2-mrp and overall expansion of the chromatin structure (FIG. 5D). This led to the conclusion that chromatin compaction is impaired in pfap2-mrp deficient parasites resulting in increased chromatin accessibility and up-regulation of var genes and genes involved in sexual differentiation. Such an effect could explain the partial disconnect between RNA-seq and ChIP-seq data observed in our study at both 16 and 40 h.p.i.

### DISCUSSION

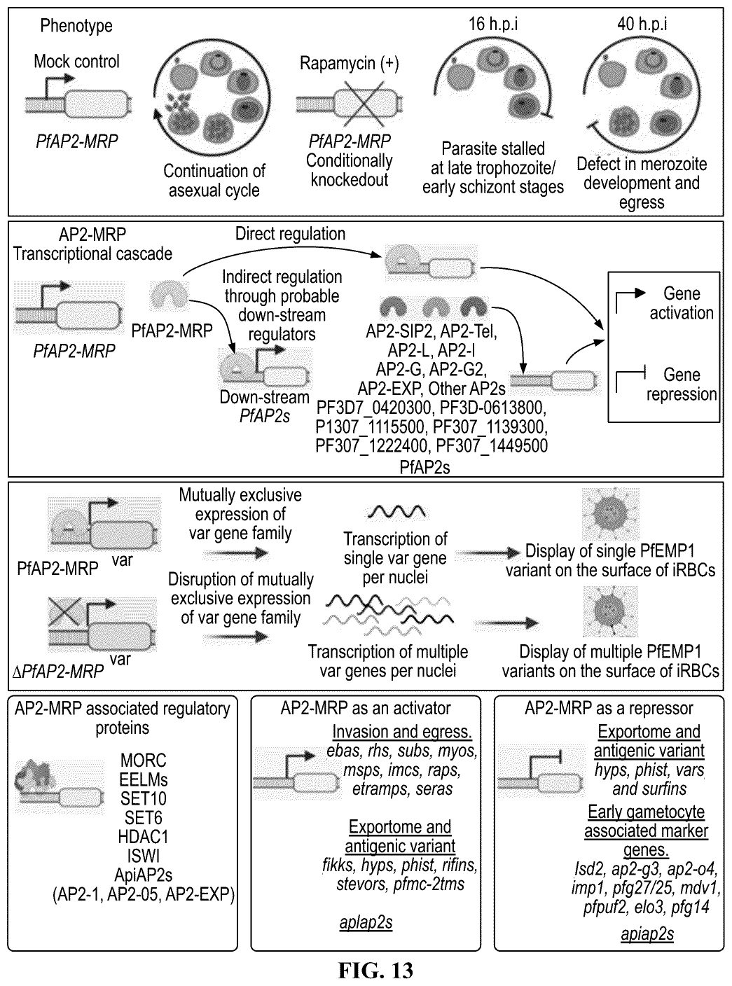

Multiple lines of evidence was provided demonstrating that PfAP2-MRP is a master regulator that controls key processes of malaria development and pathogenesis during the IDC. Functional deletion of PfAP2-MRP at time points corresponding to its two peaks of expression enabled us to show that each peak has separate essential roles. The first peak at 16 h.p.i. is critical for parasite development beyond the trophozoite stage while the second peak is indispensable for merozoite development and egress (FIG. 13). Through the use of RNA-seq, ChIP-seq and Hi-C, it was shown that that PfAP2-MRP represses vars at both 40 h.p.i. and 16 h.p.i. by either interacting with their promoter or by regulating the chromosome accessibility that might contribute to the maintenance of chromatin compaction. scRNA-seq experiments further revealed the activation of most silenced var genes at 16 h.p.i. in ΔPfAP2-MRP parasites at a single nucleus level. The FACS-based experiment with pooled immune sera from a malaria-endemic region indicated greater immune recognition of the translated transcripts of multiple var genes as PfEMP1 proteins on the surface of iRBCs in the ΔPfAP2-MRP parasites. Taken together, these results suggest that PfAP2-MRP plays a crucial role in maintaining the mutually exclusive expression pattern of the var gene family by silencing most var genes. Regulation of var gene expression involves many chromatin-associated proteins³⁴, and many of them were found to be associated with PfAP2-MRP, suggesting that it is a recruiter of these proteins.

In this study a few novel chromatin remodelers and histone modifiers were identified in complex with PfAP2-P, supporting the idea that PfAP2-P recruits these epigenetic regulators to regulate var and other virulence gene expression through chromatin organization. These proteins include PfMORC, EELM2 domain-containing proteins, imitation switch (ISW1), HDAC1-a class I-type histone deacetylase and chromodomain-helicase-DNA binding domain-containing proteins. Identification of PfEELM2 and PfMORC bound to PfAP2-P is consistent with results from a P. berghei study in which PBANKA_0939100, the ortholog of PfAP2-P, was shown to interact with PbEELM2 (PBANKA_1234600) and PbMORC (PBANKA_1331400), in schizonts. EELM2 and MORC proteins are often associated with histone deacetylase in a complex with chromatin remodelling activities associated with gene suppression. MORC proteins have the capability to topologically constrain DNA to facilitate gene silencing via chromatin compaction. RecentlyToxoplasma gondii MORC was shown to be a transcriptional repressor of sexual ommitment when interacting with histone deacetylase (HDAC3) and AP2 DNA-binding proteins. In this study, no association of PfHDAC3 with PfAP2-P was observed. However, association of PfHDAC1 with PfAP2-P was observedat both 16 and 40 h.p.i. PfMORC and PfHDAC1 have been shown to have two expression peaks in the IDC, coincident with those of PfAP2-P (Pearson correlation>0.7). It is therefore possible that PfAP2-P interacts with PfMORC and PfHDAC1 to repress var gene expression.

A weak or no association of HP1 with PfAP2-P at 16 h.p.i and 40 h.p.i. respectively was observed, and no association of PfSIR2A or 2B with PfAP2-P at both 16 and 40 h.p.i. No depletion of the H3K9me3 mark or enrichment of H3K9ac and H3K4me3 marks in Δpfap2-p parasites was observed, which are linked to HP1, PfSIR2A and 2B involvement. Therefore, it appears PfAP2-P together with other chromatin regulators and histone modifiers act independent of HP1 and the reported histone modifications (i.e. H3K9me3, H3K9ac and H3K4me3) to either suppress and/or activate var, other antigenic variant protein genes and sub-telomeric heterochromatin-associated genes.

Disruption of PfAP2-MRP expression at 40 h.p.i. was sufficient to down-regulate genes associated with merozoite development, egress and invasion processes. Using mass spectrometry, the current study and others have demonstrated that PfAP2-MRP forms complexes with other Api-AP2s and chromatin-associated proteins, indicating that PfAP2-MRP-regulated genes may be under the combinatorial control of multiple regulatory factors. It was shown that PfAP2-MRP and PfAP2-I30 bind to the same promoter regions of many invasion and egress-associated genes. The combined results from RNA-seq and ChIP-seq experiments as well as IP-mass spectrometry indicate that a positive feedback loop-based transcriptional regulatory network exists between these two transcription factors. Over-expression of many early gametocyte marker genes was observed in Δpfap2-mrp parasites in both 16 and 40 h.p.i. stages. This up-regulation of sexual genes may be explained by the decrease in heterochromatin clustering as well as a direct interaction with critical AP2 transcription factors. Overall, the result indicates that as an essential positive regulator of asexual growth, PfAP2-MRP has evolved to prevent sexual commitment by repressing early gametocyte marker genes critical for sexual conversion.

In addition to enrichment of egress and invasion-associated genes, observed were many genes encoding hypothetical proteins of unknown function in the list of most down-regulated genes at 40 h.p.i. (n=50). Based on the principle of ‘guilt-by-association’, this shows that most of the top down-regulated genes encode hypothetical proteins that are previously unidentified components of the egress and invasion pathways. For example, of note is that four of these genes encode hypothetical proteins (Pf3D7_1014100, Pf3D7_0210600, PF3D7_0308300, PF3D7_0507400) that have recently been shown to be important in invasion and egress pathways⁴¹⁻⁴⁴ are also down regulated in our study.

In conclusion, this study establishes that several important processes in malaria development and pathogenicity are controlled by a master regulator, PfAP2-MRP. This paves the way new therapeutic strategies, such as the use of Δpfap2-mrp parasites as a live anti-disease vaccine that expresses most of the PfEMP1 repertoire to elicit antibody responses that will decrease severe malaria-associated disease.

The ChIP-seq data and motif enrichment analysis of the current study indicate that PfAP2-MRP binds to the same regions of many of these genes, suggesting a complex gene regulatory system, important for both asexual parasite growth and sexual commitment. For example, a combinatorial binding of transcription factors to the same promoter has been proposed as a mechanism to increase specificity and facilitate fine-tuning⁷⁶. The binding of PfAP2-MRP and other PfAP2 transcription factors to the same promoter regions is consistent with such a mechanism. A recent study⁷⁷, has identified that ApiAP2s are regulated by multiple other ApiAP2s. A couple of such cases are pfap2-hc and PF3D7_0613800 (another uncharacterized ApiAP2), which are regulated by at least 8 ApiAP2s including themselves, which further supports the role of combinatorial based gene regulation in malaria parasites.

Also identified in the current study are PfEELM2 and PfMORC associated PfAP2-MRP at both 16 and 40 h.p.i., EELM2 and MORC proteins are often associated with histone deacetylase in a complex with chromatin remodeling activities associated with gene suppression⁷⁹ It is possible that PfAP2-MRP and PfMORC interact together to repress var gene expression. Also identified are known transcriptional activators such as PfSET10, PfSET6, and PfISWI interacting with PfAP2-MRP; PfAP2-MRP may recruit histone modifiers and chromatin remodelers to alter the cis-chromatin structure, leading to either gene activation or repression. This would be consistent with roles for PfAP2-MRP as both activator (of some genes associated with antigenic variation, host cell modification, egress and invasion) and repressor (of var and gametocytogenesis-associated genes).

Signal transduction pathways that rely on protein kinases and phosphatases have crucial roles in the parasite life cycle^{82,83} including the merozoite egress and erythrocyte invasion stages where there are substantial differences in the phosphoproteomes of intracellular schizonts and extracellular merozoites⁸⁴. Many of the proteins phosphorylated in merozoites probably participate in egress, movement, and invasion, and are phosphorylated by kinases such as PKG, PKA, CDPK1, and CDPK5. Genes encoding these kinases were down-regulated in Δpfap2-mrp parasites at 40 h.p.i., suggesting another significant role for PfAP2-MRP in mechanisms regulating these pathogenic processes.

Increased expression of many known and putative gametocyte-marker genes was observed in Δpfap2-mrp parasites at both 16 and 40 h.p.i., including genes encoding recently identified putative transcriptional regulators of gametocytogenesis such as lysine-specific demethylase (LSD2, a putative histone demethylase), AP2-04, AP2-G3, and AP2 (PF3D7_1139300)⁸⁹. LSD2 and AP2 were identified as potential regulators driving the expression of genes for gametocyte development in committed schizonts⁹⁰. AP2-G3, strongly up-regulated in Δpfap2-mrp parasites, probably plays an essential role in gametocyte production as a regulator upstream of AP2-G⁹¹. Other up-regulated genes include those for male gametocyte development (PfMDV-1) and an mRNA binding protein (PfPuf2) important in male and female gametocyte development, respectively^{92,93}. These data suggest that PfAP2-MRP inhibits commitment to sexual stage development acting through an indirect regulator no binding of PfAP2-MRP to the promoters of these genes was observed.

### REFERENCES

1 Cowman, A. F., Tonkin, C. J., Tham, W. H. & Duraisingh, M. T. The Molecular Basis of Erythrocyte Invasion by Malaria Parasites. Cell Host Microbe 22, 232-245, doi:10.1016/j.chom.2017.07.003 (2017).
2 Boddey, J. A. & Cowman, A. F. Plasmodium nesting: remaking the erythrocyte from the inside out. Annu Rev Microbiol 67, 243-269, doi:10.1 146/annurev-micro-092412-155730 (2013).
3 Tan, M. S. Y. & Blackman, M. J. Malaria parasite egress at a glance. J Cell Sci 134, doi:10.1242/jcs.257345 (2021).
4 Cortes, A. & Deitsch, K. W. Malaria Epigenetics. Cold Spring Harb Perspect Med 7, doi:10.1101/cshperspect.a025528 (2017).
Toenhake, C. G. et al. Chromatin Accessibility-Based Characterization of the Gene Regulatory Network Underlying Plasmodium falciparum Blood-Stage Development. Cell Host Microbe 23, 557-569 e559, doi:10.1016/j.chom.2018.03.007 (2018).
6 Iwanaga, S., Kaneko, I., Kato, T. & Yuda, M. Identification of an AP2-family protein that is critical for malaria liver stage development. PLoS One 7, e47557, doi:10.1371/journal.pone.0047557 (2012).
7 Kafsack, B. F. et al. A transcriptional switch underlies commitment to sexual development in malaria parasites. Nature 507, 248-252, doi:10.1038/naturel2920 (2014).
8 Painter, H. J., Campbell, T. L. & Llinas, M. The Apicomplexan AP2 family: integral factors regulating Plasmodium development. Mol Biochem Parasitol 176, 1-7, doi:10.1016/j.molbiopara.2010.11.014 (2011).
9 Tinto-Font, E. et al. A heat-shock response regulated by the PfAP2-HS transcription factor protects human malaria parasites from febrile temperatures. Nat Microbiol 6, 1163-1174, doi:10.1038/s41564-021-00940-w (2021).
10 Yuda, M., Iwanaga, S., Shigenobu, S., Kato, T. & Kaneko, I. Transcription factor AP2-Sp and its target genes in malarial sporozoites. Mol Microbiol 75, 854-863, doi:10.1111/j.1365-2958.2009.07005.x (2010).
11 Yuda, M. et al. Identification of a transcription factor in the mosquito-invasive stage of malaria parasites. Mol Microbiol 71, 1402-1414, doi:10.1111/j.1365-2958.2009.06609.x (2009).
12 Collins, C. R. et al. Malaria parasite cGMP-dependent protein kinase regulates blood stage merozoite secretory organelle discharge and egress. PLoS Pathog 9, e1003344, doi:10.1371/journal.ppat.1003344 (2013).
13 Knuepfer, E., Napiorkowska, M., van Ooij, C. & Holder, A. A. Generating conditional gene knockouts in Plasmodium—a toolkit to produce stable DiCre recombinase-expressing parasite lines using CRISPR/Cas9. Sci Rep 7, 3881, doi:10.1038/s41598-017-03984-3 (2017).
14 Subudhi, A. K. et al. Malaria parasites regulate intra-erythrocytic development duration via serpentine receptor 10 to coordinate with host rhythms. Nat Commun 11, 2763, doi:10.1038/s41467-020-16593-y (2020).
15 Gomes, A. R. et al. A genome-scale vector resource enables high-throughput reverse genetic screening in a malaria parasite. Cell Host Microbe 17, 404-413, doi:10.1016/j.chom.2015.01.014 (2015).
16 Zhang, M. et al. Uncovering the essential genes of the human malaria parasite Plasmodium falciparum by saturation mutagenesis. Science 360, doi:10.1 126/science.aap7847 (2018).
17 Thomas, J. A. et al. A protease cascade regulates release of the human malaria parasite Plasmodium falciparum from host red blood cells. Nat Microbiol 3, 447-455, doi:10.1038/s41564-018-0111-0 (2018).
18 Scherf, A., Lopez-Rubio, J. J. & Riviere, L. Antigenic variation in Plasmodium falciparum. Annu Rev Microbiol 62, 445-470, doi:10.1 146/annurev.micro.61.080706.093134 (2008).
19 Baker, D. A. et al. Cyclic nucleotide signalling in malaria parasites. Open Biol 7, doi:10.1098/rsob.170213 (2017).
20 Singh, S. & Chitnis, C. E. Molecular Signaling Involved in Entry and Exit of Malaria Parasites from Host Erythrocytes. Cold Spring Harb Perspect Med 7, doi:10.1101/cshperspect.a026815 (2017).
21 Sargeant, T. J. et al. Lineage-specific expansion of proteins exported to erythrocytes in malaria parasites. Genome Biol 7, R12, doi:10.1186/gb-2006-7-2-r12 (2006).
22 Siddiqui, G., Proellochs, N. I. & Cooke, B. M. Identification of essential exported Plasmodium falciparum protein kinases in malaria-infected red blood cells. Br J Haematol 188, 774-783, doi:10.1111/bjh.16219 (2020).
23 Howick, V. M. et al. The Malaria Cell Atlas: Single parasite transcriptomes across the complete Plasmodium life cycle. Science 365, doi:10.1 126/science.aaw2619 (2019).
24 Taylor, T. E. et al. Intravenous immunoglobulin in the treatment of paediatric cerebral malaria. Clin Exp Immunol 90, 357-362, doi:10.1111/j.1365-2249.1992.tb05851.x (1992).
25 Chan, J. A., Fowkes, F. J. & Beeson, J. G. Surface antigens of Plasmodium falciparum-infected erythrocytes as immune targets and malaria vaccine candidates. Cell Mol Life Sci 71, 3633-3657, doi:10.1007/s00018-014-1614-3 (2014).
26 Gulati, S. et al. Profiling the Essential Nature of Lipid Metabolism in Asexual Blood and Gametocyte Stages of Plasmodium falciparum. Cell Host Microbe 18, 371-381, doi:10.1016/j.chom.2015.08.003 (2015).
27 Josling, G. A. & Llinas, M. Sexual development in Plasmodium parasites: knowing when it's time to commit. Nat Rev Microbiol 13, 573-587, doi:10.1038/nrmicro3519 (2015).
28 Poran, A. et al. Single-cell RNA sequencing reveals a signature of sexual commitment in malaria parasites. Nature 551, 95-99, doi:10.1038/nature24280 (2017).
29 Shang, X. et al. Genome-wide landscape of ApiAP2 transcription factors reveals a heterochromatin-associated regulatory network during Plasmodium falciparum blood-stage development. Nucleic Acids Res, doi:10.1093/nar/gkac176 (2022).
30 Santos, J. M. et al. Red Blood Cell Invasion by the Malaria Parasite Is Coordinated by the PfAP2-I Transcription Factor. Cell Host Microbe 21, 731-741 e710, doi:10.1016/j.chom.2017.05.006 (2017).
31 Josling, G. A. et al. Dissecting the role of PfAP2-G in malaria gametocytogenesis. Nat Commun 11, 1503, doi:10.1038/s41467-020-15026-0 (2020).
32 Volz, J. C. et al. PfSET10, a Plasmodium falciparum methyltransferase, maintains the active var gene in a poised state during parasite division. Cell Host Microbe 11, 7-18, doi:10.1016/j.chom.2011.11.011 (2012).
33 Volz, J. et al. Potential epigenetic regulatory proteins localise to distinct nuclear sub-compartments in Plasmodium falciparum. Int J Parasitol 40, 109-121, doi:10.1016/j.ijpara.2009.09.002 (2010).
34 Bryant, J. M. et al. Exploring the virulence gene interactome with CRISPR/dCas9 in the human malaria parasite. Mol Syst Biol 16, e9569, doi:10.15252/msb.20209569 (2020).
35 Farhat, D. C. et al. A MORC-driven transcriptional switch controls Toxoplasma developmental trajectories and sexual commitment. Nat Microbiol 5, 570-583, doi:10.1038/s41564-020-0674-4 (2020).
36 Andrews, K. T., Haque, A. & Jones, M. K. HDAC inhibitors in parasitic diseases. Immunol Cell Biol 90, 66-77, doi:10.1038/icb.2011.97 (2012).
37 Ay, F. et al. Three-dimensional modeling of the P. falciparum genome during the erythrocytic cycle reveals a strong connection between genome architecture and gene expression. Genome Res 24, 974-988, doi:10.1101/gr.169417.113 (2014).
38 Bunnik, E. M. et al. Changes in genome organization of parasite-specific gene families during the Plasmodium transmission stages. Nat Commun 9, 1910, doi:10.1038/s41467-018-04295-5 (2018).
39 Bunnik, E. M. et al. Comparative 3D genome organization in apicomplexan parasites. Proc Natl Acad Sci USA 116, 3183-3192, doi:10.1073/pnas.1810815116 (2019).
40 Oliver, S. Guilt-by-association goes global. Nature 403, 601-603, doi:10.1038/35001165 (2000).
41 Nagaoka, H. et al. PfMSA180 is a novel Plasmodium falciparum vaccine antigen that interacts with human erythrocyte integrin associated protein (CD47). Sci Rep 9, 5923, doi:10.1038/s41598-019-42366-9 (2019).
42 Liffner, B. et al. PfCERLI1 is a conserved rhoptry associated protein essential for Plasmodium falciparum merozoite invasion of erythrocytes. Nat Commun 11, 1411, doi:10.1038/s41467-020-15127-w (2020).
43 Wichers, J. S. et al. Identification of novel inner membrane complex and apical annuli proteins of the malaria parasite Plasmodium falciparum. Cell Microbiol 23, e13341, doi:10.1111/cmi.13341 (2021).
44 Tarr, S. J. et al. A malaria parasite subtilisin propeptide-like protein is a potent inhibitor of the egress protease SUB1. Biochem J 477, 525-540, doi:10.1042/BCJ20190918 (2020).
45 Moon, R. W. et al. Adaptation of the genetically tractable malaria pathogen Plasmodium knowlesi to continuous culture in human erythrocytes. Proc Natl Acad Sci USA 110, 531-536, doi:10.1073/pnas.1216457110 (2013).
46 Jones, M. L. et al. A versatile strategy for rapid conditional genome engineering using loxP sites in a small synthetic intron in Plasmodium falciparum. Sci Rep 6, 21800, doi: 10. 1038/srep21800 (2016).
47 Bolger, A. M., Lohse, M. & Usadel, B. Trimmomatic: a flexible trimmer for Illumina sequence data. Bioinformatics 30, 2114-2120, doi:10.1093/bioinformatics/btu170 (2014).
48 Kim, D., Langmead, B. & Salzberg, S. L. HISAT: a fast spliced aligner with low memory requirements. Nat Methods 12, 357-360, doi:10.1038/nmeth.3317 (2015).
49 Liao, Y., Smyth, G. K. & Shi, W. featureCounts: an efficient general purpose program for assigning sequence reads to genomic features. Bioinformatics 30, 923-930, doi:10.1093/bioinformatics/btt656 (2014).
50 McCarthy, D. J., Chen, Y. & Smyth, G. K. Differential expression analysis of multifactor RNA-Seq experiments with respect to biological variation. Nucleic Acids Res 40, 4288-4297, doi:10.1093/nar/gks042 (2012).
51 Ritchie, M. E. et al. limma powers differential expression analyses for RNA-sequencing and microarray studies. Nucleic Acids Res 43, e47, doi:10.1093/nar/gkv007 (2015).
52 Love, M. I., Huber, W. & Anders, S. Moderated estimation of fold change and dispersion for RNA-seq data with DESeq2. Genome Biol 15, 550, doi:10.1186/s13059-014-0550-8 (2014).
53 Pfaffl, M. W. A new mathematical model for relative quantification in real-time RT-PCR. Nucleic Acids Res 29, e45, doi:10.1093/nar/29.9.e45 (2001).
54 Jiang, L. et al. PfSETvs methylation of histone H3K36 represses virulence genes in Plasmodium falciparum. Nature 499, 223-227, doi:10.1038/nature12361 (2013).
55 Zeeshan, M. et al. Real-time dynamics of Plasmodium NDC80 reveals unusual modes of chromosome segregation during parasite proliferation. J Cell Sci 134, doi:10.1242/jcs.245753 (2020).
56 Li, H. et al. The Sequence Alignment/Map format and SAMtools. Bioinformatics 25, 2078-2079, doi:10.1093/bioinformatics/btp352 (2009).
57 Ramirez, F., Dundar, F., Diehl, S., Gruning, B. A. & Manke, T. deepTools: a flexible platform for exploring deep-sequencing data. Nucleic Acids Res 42, W187-191, doi:10.1093/nar/gku365 (2014).
58 Thorvaldsdottir, H., Robinson, J. T. & Mesirov, J. P. Integrative Genomics Viewer (IGV): high-performance genomics data visualization and exploration. Brief Bioinform 14, 178-192, doi:10.1093/bib/bbs017 (2013).
59 Zhang, Y. et al. Model-based analysis of ChIP-Seq (MACS). Genome Biol 9, R137, doi:10.1186/gb-2008-9-9-r137 (2008).
60 Quinlan, A. R. & Hall, I. M. BEDTools: a flexible suite of utilities for comparing genomic features. Bioinformatics 26, 841-842, doi:10.1093/bioinformatics/btq033 (2010).
61 Lun, A. T. L. et al. EmptyDrops: distinguishing cells from empty droplets in droplet-based single-cell RNA sequencing data. Genome Biol 20, 63, doi:10.1186/s13059-019-1662-y (2019).
62 McCarthy, D. J., Campbell, K. R., Lun, A. T. & Wills, Q. F. Scater: pre-processing, quality control, normalization and visualization of single-cell RNA-seq data in R. Bioinformatics 33, 1179-1186, doi:10.1093/bioinformatics/btw777 (2017).
63 Lun, A. T., McCarthy, D. J. & Marioni, J. C. A step-by-step workflow for low-level analysis of single-cell RNA-seq data with Bioconductor. F1000Res 5, 2122, doi:10.12688/f1000research.9501.2 (2016).
64 Hao, Y. et al. Integrated analysis of multimodal single-cell data. Cell 184, 3573-3587 e3529, doi:10.1016/j.cell.2021.04.048 (2021).
65 Real, E. et al. A single-cell atlas of Plasmodium falciparum transmission through the mosquito. Nat Commun 12, 3196, doi:10.1038/s41467-021-23434-z (2021).
66 Aran, D. et al. Reference-based analysis of lung single-cell sequencing reveals a transitional profibrotic macrophage. Nat Immunol 20, 163-172, doi:10.1038/s41590-018-0276-y (2019).
67 de Kanter, J. K., Lijnzaad, P., Candelli, T., Margaritis, T. & Holstege, F. C. P. CHETAH: a selective, hierarchical cell type identification method for single-cell RNA sequencing. Nucleic Acids Res 47, e95, doi:10.1093/nar/gkz543 (2019).
68 Bailey, T. L. DREME: motif discovery in transcription factor ChIP-seq data. Bioinformatics 27, 1653-1659, doi:10.1093/bioinformatics/btr261 (2011).
69 Gupta, S., Stamatoyannopoulos, J. A., Bailey, T. L. & Noble, W. S. Quantifying similarity between motifs. Genome Biol 8, R24, doi:10.1186/gb-2007-8-2-r24 (2007).
70 Campbell, T. L., De Silva, E. K., Olszewski, K. L., Elemento, O. & Llinas, M. Identification and genome-wide prediction of DNA binding specificities for the ApiAP2 family of regulators from the malaria parasite. PLoS Pathog 6, e1001165, doi:10.1371/journal.ppat.1001165 (2010).
71 Rao, S. S. et al. A 3D map of the human genome at kilobase resolution reveals principles of chromatin looping. Cell 159, 1665-1680, doi:10.1016/j.cell.2014.11.021 (2014).
72 Gupta, M. K., Lenz, T. & Le Roch, K. G. Chromosomes Conformation Capture Coupled with Next-Generation Sequencing (Hi-C) in Plasmodium falciparum. Methods Mol Biol 2369, 15-25, doi:10.1007/978-1-0716-1681-9_2 (2021).
73 Varoquaux, N., Ay, F., Noble, W. S. & Vert, J. P. A statistical approach for inferring the 3D structure of the genome. Bioinformatics 30, i26-33, doi:10.1093/bioinformatics/btu268 (2014).
74 Goddard, T. D. et al. UCSF ChimeraX: Meeting modern challenges in visualization and analysis. Protein Sci 27, 14-25, doi:10.1002/pro.3235 (2018).
75 Josling, G. A. et al. Dissecting the role of PfAP2-G in malaria gametocytogenesis. Nat Commun 11, 1503, doi:10.1038/s41467-020-15026-0 (2020).
76 Reiter, F., Wienerroither, S. & Stark, A. Combinatorial function of transcription factors and cofactors. Curr Opin Genet Dev 43, 73-81, doi:10.1016/j.gde.2016.12.007 (2017).
77 Shang, X. et al. A cascade of transcriptional repression determines sexual commitment and development in Plasmodium falciparum. Nucleic Acids Res 49, 9264-9279, doi:10.1093/nar/gkab683 (2021).
78 Bryant, J. M. et al. Exploring the virulence gene interactome with CRISPR/dCas9 in the human malaria parasite. Mol Syst Biol 16, e9569, doi:10.15252/msb.20209569 (2020).
79 Solari, F., Bateman, A. & Ahringer, J. The Caenorhabditis elegans genes egl-27 and egr-1 are similar to MTA1, a member of a chromatin regulatory complex, and are redundantly required for embryonic patterning. Development 126, 2483-2494 (1999).
80 Farhat, D. C. et al. A MORC-driven transcriptional switch controls Toxoplasma developmental trajectories and sexual commitment. Nat Microbiol 5, 570-583, doi:10.1038/s41564-020-0674-4 (2020).
81 Subudhi, A. K. et al. Malaria parasites regulate intra-erythrocytic development duration via serpentine receptor 10 to coordinate with host rhythms. Nat Commun 11, 2763, doi:10.1038/s41467-020-16593-y (2020).
82 Baker, D. A. et al. Cyclic nucleotide signalling in malaria parasites. Open Biol 7, doi:10.1098/rsob.170213 (2017).
83 Singh, S. & Chitnis, C. E. Molecular Signaling Involved in Entry and Exit of Malaria Parasites from Host Erythrocytes. Cold Spring Harb Perspect Med 7, doi:10.1 101/cshperspect.a026815 (2017).
84 Lasonder, E., Green, J. L., Grainger, M., Langsley, G. & Holder, A. A. Extensive differential protein phosphorylation as intraerythrocytic Plasmodium falciparum schizonts develop into extracellular invasive merozoites. Proteomics 15, 2716-2729, doi:10.1002/pmic.201400508 (2015).
85 Proellocks, N. I., Coppel, R. L., Mohandas, N. & Cooke, B. M. Malaria Parasite Proteins and Their Role in Alteration of the Structure and Function of Red Blood Cells. Adv Parasitol 91, 1-86, doi:10.1016/bs.apar.2015.09.002 (2016).
86 Ward, P., Equinet, L., Packer, J. & Doerig, C. Protein kinases of the human malaria parasite Plasmodium falciparum: the kinome of a divergent eukaryote. BMC Genomics 5, 79, doi:10.1186/1471-2164-5-79 (2004).
87 Davies, H. et al. An exported kinase family mediates species-specific erythrocyte remodelling and virulence in human malaria. Nat Microbiol 5, 848-863, doi:10.1038/s41564-020-0702-4 (2020).
88 Siddiqui, G., Proellochs, N. I. & Cooke, B. M. Identification of essential exported Plasmodium falciparum protein kinases in malaria-infected red blood cells. Br J Haematol 188, 774-783, doi:10.1111/bjh.16219 (2020).
89 Zhang, M. et al. Uncovering the essential genes of the human malaria parasite Plasmodium falciparum by saturation mutagenesis. Science 360, doi:10.1 126/science.aap7847 (2018).
90 Poran, A. et al. Single-cell RNA sequencing reveals a signature of sexual commitment in malaria parasites. Nature 551, 95-99, doi:10.1038/nature24280 (2017).
91 Josling, G. A. & Llinas, M. Sexual development in Plasmodium parasites: knowing when it's time to commit. Nat Rev Microbiol 13, 573-587, doi:10.1038/nrmicro3519 (2015).
92 Furuya, T. et al. Disruption of a Plasmodium falciparum gene linked to male sexual development causes early arrest in gametocytogenesis. Proc Natl Acad Sci USA 102, 16813-16818, doi:10.1073/pnas.0501858102 (2005).
93 Miao, J. et al. The Puf-family RNA-binding protein PfPuf2 regulates sexual development and sex differentiation in the malaria parasite Plasmodium falciparum. J Cell Sci 123, 1039-1049, doi:10.1242/jcs.059824 (2010).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. An immunogenic composition comprising an effective amount of
(i) a protein encoded by any of the genes of Table 1 or Table 2, a variant thereof with at least 75% sequence identity thereto, or a fragment thereof;
(ii) a nucleic acid encoding the protein or variant or fragment of (i); or
(iii) comprising live attenuated ΔPFAP2-MRP (LA-ΔPFAP2-MRP) Plasmodium sp. in a pharmaceutically acceptable carrier.

2. The immunogenic composition of claim 1 comprising (ii), wherein the nucleic acid is mRNA or a vector.

3. The immunogenic composition of claim 1, wherein the Plasmodium sp. is selected from the group consisting of P. malariae, P. vivax, Plasmodium falciparum, P. ovale; and P. knowlesi.

4. The immunogenic composition of claim 1, further comprising an adjuvant.

5. A pharmaceutical composition comprising an effective amount of an inhibitor of
(i) a protein encoded by any of the genes of Table 1 or Table 2, a variant thereof with at least 75% sequence identity thereto, or a fragment thereof; or
(ii) a nucleic acid encoding the protein or variant or fragment of (i).

6. The composition of claim 5, wherein the inhibitor is a functional nucleic acid, or an expression vector encoding a functional nucleic acid.

7. The composition of claim 5, wherein the inhibitor is a small molecule.

8. A method of treating a subject in need thereof comprising administering the subject an effective amount of the composition of any one of claims 6-8 to reduce one or more symptoms of a parasitic infection.

9. The method of claim 8, wherein the subject has malaria, optionally, the method further comprising administering the subject second anti-malarial agent.

10. The method of claim 8, wherein the subject has a Plasmodium falciparum infection.

11. The composition of claim 1, comprising: (a) LA-ΔPFAP2-MRP Plasmodium falciparum; (b) sporozoite stage LA-ΔPFAP2-MRP Plasmodium; and/or (c) merozoites stage LA-ΔPFAP2-MRP Plasmodium.

12. The composition of claim 1 wherein the LA-ΔPFAP2-MRP Plasmodium has been chemically, optionally, wherein the LA-ΔPFAP2-MRP Plasmodium is an irradiated LA-ΔPFAP2-MRP Plasmodium or genetically attenuated.

13. A method of inducing an immune response in a subject comprising administering the subject an effective amount of the immunogenic composition of claim 1 to induce an immune response against the protein or variant or fragment.

14. The method of claim 13, wherein administration of the one or more doses is not followed by administration of an antimalarial agent that is capable of preventing/blocking Plasmodium growth.

15. The method of claim 13, comprising administering one or more doses of the composition, wherein the one or more doses comprise no more than 150,000 sporozoites.

16. The method of claim 13 comprising administering one or more doses of the composition, wherein said one or more doses comprise no more than 50,000 sporozoites.

17. The method of claim 13, comprising administering one or more doses of the composition, wherein said one or more doses comprise no more than 25,000 sporozoites.

18. The method of claim 13, wherein no more than 3, 2 or 1 dose is administered.

19. The method of claim 13, wherein the Plasmodium sp. is selected from the group consisting of: P. falciparum, P. vivax, P. ovale, P. knowlesi, and P. malariae.

20. The method of claim 19, wherein the Plasmodium sp. is P. falciparum.
